(19) United States
(12) Patent Application Publication
(10) Pub. No.: US 2006/0111289 A1
(43) Pub. Date: May 25, 2006

(54) Compositions and methods of using Alpha-fetoprotein growth inhibitory peptides

(75) Inventor: Gerald J. Mizejewski, Clifton Park, NY (US)

Correspondence Address: MEDLEN & CARROLL, LLP;Suite 350, 101 Howard Street, San Francisco, CA, 94105, US

(73) Assignee: Serometrix, LLC,, Syracuse, NY (US)

(21) Appl. No.: 11/223,610

(22) Filed: Sep. 9, 2005

Related U.S. Application Data
(60) Provisional application No. 60/608,393, filed on Sep. 9, 2004.

(51) Int. Cl. A61K 38/54; A61K 38/18; C07K 14/475

(52) U.S. Cl. 514/12; 514/13; 530/324; 530/326

(57) ABSTRACT

This invention describes compositions related to growth inhibitory protein-derived peptide fragments. These fragments can be identified by several methods including homology comparison and amino acid pairing techniques. Certain fragment sequences have been shown to provide homology to various biological activities and are contemplated as potential therapeutic agents. These biological activities include, but are not limited to, anti-cancer effects, nonselective calcium channel regulation, angiogenesis inhibition, cytoskeletal controls, cell cycle regulation, enzyme function, transcription, and anti-microbials.

### FIELD OF INVENTION

The present invention is related to an α-fetoprotein (AFP) derived peptide termed the Growth Inhibitory Peptide (GIP). In one embodiment, GIP comprises consensus amino acid sequences modulating a variety of biological activities. In one embodiment, the modulated biological activities include, but are not limited to, anti-cancer effects, nonselective calcium channel regulation, angiogenesis inhibition, cytoskeletal controls, cell cycle regulation, enzyme function, transcription, and anti-microbials.

### BACKGROUND OF THE INVENTION

α-fetoprotein (AFP) is a known mammalian fetal protein and recognized tumor marker. The structure-function relationships of AFP has been investigated during the last decade. Study of biological and physiological activities of these peptides allows determining biologically active sites of α-fetoprotein and constructing a possible structural and functional map. One suggested therapeutic use for AFP includes anticancer therapy.

Conformational changes in the AFP molecule have been identified demonstrating significant conformational mobility while retaining stability in solution. One hypothesis suggests that a native AFP protein may contain cryptic biologically active sites which are not available for ligand binding until AFP changes into a specific conformation.

Conformational changes are, of course, based upon the primary amino acid sequence of a protein. Modifications to a native, or wild-type, amino acid sequence would therefore be expected to modify a peptide's biological activity. Consequently, overlooked AFP compositions and alternative uses is likely because novel AFP-derived amino acid sequences have not been reported or studied.

What is needed are novel AFP-derived amino acid sequences that demonstrate a wide variety of biological activities useful in the treatment of various disease states.

### SUMMARY OF THE INVENTION

The present invention is related to an α-fetoprotein (AFP) derived peptide termed the Growth Inhibitory Peptide (GIP). In one embodiment, GIP comprises consensus amino acid sequences modulating a variety of biological activities. In one embodiment, the modulated biological activities include, but are not limited to, anti-cancer effects, nonselective calcium channel regulation, angiogenesis inhibition, cytoskeletal controls, cell cycle regulation, enzyme function, transcription, and anti-microbials.

In one embodiment, the present invention contemplates a growth inhibitory protein-derived fragment and/or subfragment comprising homology to SEQ ID NO:2. In one embodiment, the fragment comprises a matrix metalloproteinase-associated peptide comprising at least a portion of LSX₁DX₂X₃X₄ACGEGX₅AX₆IX₇X₈GHX₉X₁₀X₁₁RHX₁₂X₁₃X₁₄PX₁₅X₁₆-PGVG (SEQ ID NO:75). In one embodiment, X₁ is selected from the group consisting of E or D. In one embodiment, X₂ is selected from the group consisting of K, R, or L. In one embodiment, X₃ is selected from the group consisting of L or H. In one embodiment, X₄ is selected from the group consisting of L or M. In one embodiment, X₅ is selected from the group consisting of A, S, or I. In one embodiment, X₆ is selected from the group consisting of D, E, or S. In one embodiment, X₇ is selected from the group consisting of I or V. In one embodiment, X₈ is selected from the group consisting of I or G. In one embodiment, X₉ is selected from the group consisting of L or E. In one embodiment, X₁₀ is selected from the group consisting of C or W. In one embodiment, X₁₁ is selected from the group consisting of I or V. In one embodiment, X₁₂ is selected from the group consisting of E or N. In one embodiment, X₁₃ is selected from the group consisting of M, I, or P. In one embodiment, X₁₄ is selected from the group consisting of T, V, or I. In one embodiment, X₁₅ is selected from the group consisting of V, E, or Y. In one embodiment, X₁₆ is selected from the group consisting of N, G, or F. In one embodiment, the fragment comprises an extracellular matrix-associated peptide comprising at least a portion of LSEX₁KLLX₂CGX₃GX₄X₅X₆IX₇X₈X₉HLX₁₀IX₁₁HX₁₂X₁₃X₁₄PX₁₅X₁₆PGVG (SEQ ID NO:74). In one embodiment, X₁ is selected from the group consisting of D, I, Q, R, or T. In one embodiment, X₂ is selected from the group consisting of G, A, F, or P. In one embodiment, X₃ is selected from the group consisting of E, R, S, or D. In one embodiment, X₄ is selected from the group consisting of A, G, L, V, S, or Y. In one embodiment, X₅ is selected from the group consisting of A, G, L, P, S, T, or V. In one embodiment, X₆ is selected from the group consisting of D, T, E, A, or N. In one embodiment, X₇ is selected from the group consisting of I, F, or V. In one embodiment, X₈ is selected from the group consisting of I, Y, L, V, or E. In one embodiment, X₉ is selected from the group consisting of G, L, R, or E. In one embodiment, X₁₀ is selected from the group consisting of C, V, I, or P. In one embodiment, X₁₁ is selected from the group consisting of R or K; X₁₂=E, Q, or R. In one embodiment, X₁₃ is selected from the group consisting of M, L, A, I, G, P, or F. In one embodiment, X₁₄ is selected from the group consisting of T, S, P, A, R, or I. In one embodiment, X₁₅ is selected from the group consisting of V, L, C, F, S, Y, or I. In one embodiment, X₁₆ is selected from the group consisting of N, A, L, P, or G. In one embodiment, the fragment comprises a clotting and/or adhesion-associated peptide comprising at least a portion of X₁X₂LX₃CX₄X₅GX₆X₇X₈X₉X₁₀X₁₁GHLCIRX₁₂X₁₃X_{14 -}X₁₅PX₁₆NPX₁₇X₁₈G (SEQ ID NO;76). In one embodiment, X₁ is selected from the group consisting of K or G. In one embodiment, X₂ is selected from the group consisting of E or L. In one embodiment, X₃ is selected from the group consisting of A or R. In one embodiment, X₄ is selected from the group consisting of D or G. In one embodiment, X₅ is selected from the group consisting of A, T, or E. In one embodiment, X₆ is selected from the group consisting of V, I, or T. In one embodiment, X₇ is selected from the group consisting of V, A, R, or S. In one embodiment, X₈ is selected from the group consisting of H, C, G, Q, R or D. In one embodiment, X₉ is selected from the group consisting of T, I, or V. In one embodiment, X₁₀ is selected from the group consisting of V, I, or T. In one embodiment, X₁₁ is selected from the group consisting of S, V, or I. In one embodiment, X₁₂ is selected from the group consisting of H, I, or T. In one embodiment, X₁₃ is selected from the group consisting of T, S, Q, or E. In one embodiment, X₁₄ is selected from the group consisting of N, F, N, L, or M. In one embodiment, X₁₅ is selected from the group consisting of S, G, L, Q, or T. In one embodiment, X₁₆ is selected from the group consisting of V, G, or L. In one embodiment, X₁₇ is selected from the group consisting of G, R, or A. In one embodiment, X₁₈ is selected from the group consisting of V, S, or L. In one embodiment, the fragment comprises a cation channel peptide comprising at least a portion of LSEDKLLACGEGX₁QDIIIGHX₂CIRHEMTPVNPGVG (SEQ ID NO:77). In one embodiment, X₁ is selected from the group consisting of A or D. In one embodiment, aX₂ is selected from the group consisting of L or D. In one embodiment, the cation channel comprises a calcium-stimulated potassium channel. In one embodiment, the cation channel comprises a calcium channel, In one embodiment, the cation channel comprises a potassium channel. In one embodiment, the cation channel comprises a sodium channel. In one embodiment, the fragment comprises an antiangiogenesis peptide comprising at least a portion of LSEDKLLX₁CGEX₂X₃-ADIX₄IX₅HX₆CIRHEMTPVNPX₇X₈X₉ (SEQ ID NO:81). In one embodiment, X₁ is selected from the group consisting of A or E. In one embodiment, X₂ is selected from the group consisting of G or E. In one embodiment, X₃ is selected from the group consisting of A or D. In one embodiment, X₄ is selected from the group consisting of D or I. In one embodiment, X₅ is selected from the group consisting of G or E. In one embodiment, X₆ is selected from the group consisting of L or D; In one embodiment, X₇ is selected from the group consisting of V or G. In one embodiment, X₈ is selected from the group consisting of V or G. In one embodiment, X₉ is selected from the group consisting of G or N. In one embodiment, the fragment comprises a cytoskeletal regulator peptide comprising at least a portion of LSEDKLLX₁CGEGX₂ADIIIG-HX₃CIRHEMTPVNPGV (SEQ ID NO:82). In one embodiment, X₁ is selected from the group consisting of A or E. In one embodiment, X₂ is selected from the group consisting of A or E. In one embodiment, and X₃ is selected from the group consisting of L or D. In one embodiment, the fragment comprises a cell cycle regulator peptide comprising at least a portion of X₁LLX₂CGEGAADIIIGHX₃CIRX₄EX₅TPVNPX₆X₇ (SEQ ID NO:83). In one embodiment, X₁ is selected from the group consisting of K or D. In one embodiment, X₂ is selected from the group consisting of A or E. In one embodiment, X₃ is selected from the group consisting of L or E. In one embodiment, X₄ is selected from the group consisting of H or K. In one embodiment, X₅ is selected from the group consisting of M or E. In one embodiment, X₆ is selected from the group consisting of V or D. In one embodiment, X₇ is selected from the group consisting of G or D. In one embodiment, the fragment comprises a metabolic enzyme regulator peptide comprising at least a portion of LSEDKLLACGEX₁X₂ADIIIGHX₃CIRHEMTPVNPGVG (SEQ ID NO: 84). In one embodiment, X₁ is selected from the group consisting of G or D. In one embodiment, X₂ is selected from the group consisting of A or E. In one embodiment, X₃ is selected from the group consisting of L or D. In one embodiment, the fragment comprises a transcription regulator peptide comprising at least a portion of LSEDKLLX₁CGEGAADIIIGHLCIRHEMTPVNPX₂X₃ (SEQ ID NO: 85). In one embodiment, X₁ is selected from the group consisting of A or E. In one embodiment, X₂ is selected from the group consisting of V or E. In one embodiment, X₃ is selected from the group consisting of G or no amino acid. In one embodiment, the fragment comprises a terminal protecting group, wherein said protecting group provides protease resistance. In one embodiment, the protecting group is selected from the group comprising an acyl, an amide, an acetate, a benzyl, a benzoyl group, or a D-amino acid.

In one embodiment, the present invention contemplates a method comprising i) identifying a disease specific regulatory protein; ii) matching SEQ ID NO:2 to said regulatory protein amino acid sequence; and iii) maximizing identity and similarity values, such that a growth inhibitor protein-derived peptide fragment may be synthesized based upon homology of said regulatory protein to SEQ ID NO:2. In one embodiment, the matching may be selected from the group comprising sequence reversal, D-amino acid replacement, coded amino acid pairing, non-coded amino acid pairing, or cargo bay motifs. In one embodiment, the cargo bay motifs may be selected from the group comprising nuclear transcription factors, decoy growth factors, nuclear localization signals, transforming growth factors, apoptosis FAS factors, apoptosis inhibition factors, fibroblast growth factor receptor-actor agonists, viral cloaking factors, chemokine decoy ligands, chemokine CSCR4 decoy receptors, and soluble D6 chemokine receptors.

In one embodiment, the present invention contemplates a method comprising: a) providing; i) a patient, wherein said patient exhibits at least one cancer symptom; ii) a GIP-derived peptide; and b) administering said GIP-derived peptide to said patient under conditions such that at least one said cancer symptom is reduced. In one embodiment, the GIP derived peptide is selected from the group consisting of SEQ ID NO: 74, SEQ ID NO:75, or SEQ ID NO:76.

In one embodiment, the present invention contemplates a method comprising: a) providing; i) a patient, wherein said patient exhibits at least one neurological or cardiovascular symptom related to calcium influx; ii) a GIP-derived peptide; and b) administering said GIP-derived peptide to said patient under conditions such that at least one said symptom is reduced. In one embodiment, the GIP derived peptide comprises SEQ ID NO: 77.

In one embodiment, the present invention contemplates a method comprising: a) providing; i) a patient, wherein said patient exhibits at least one symptom related to abnormal angiogenesis; ii) a GIP-derived peptide; and b) administering said GIP-derived peptide to said patient under conditions such that at least one said symptom is reduced. In one embodiment, the GIP derived peptide comprises SEQ ID NO: 81.

In one embodiment, the present invention contemplates a method comprising: a) providing; i) a patient, wherein said patient exhibits at least one symptom related to abnormal cytoskeletal modulation; ii) a GIP-derived peptide; and b) administering said GIP-derived peptide to said patient under conditions such that at least one said symptom is reduced. In one embodiment, the GIP derived peptide comprises SEQ ID NO: 82.

In one embodiment, the present invention contemplates a method comprising: a) providing; i) a patient, wherein said patient exhibits at least one symptom related to abnormal cell cycle regulation; ii) a GIP-derived peptide; and b) administering said GIP-derived peptide to said patient under conditions such that at least one said symptom is reduced. In one embodiment, the GIP derived peptide comprises SEQ ID NO: 83.

In one embodiment, the present invention contemplates a method comprising: a) providing; i) a patient, wherein said patient exhibits at least one symptom related to abnormal metabolic enzyme regulation; ii) a GIP-derived peptide; and b) administering said GIP-derived peptide to said patient under conditions such that at least one said symptom is reduced. In one embodiment, the GIP derived peptide comprises SEQ ID NO: 84.

In one embodiment, the present invention contemplates a method comprising: a) providing; i) a patient, wherein said patient exhibits at least one symptom related to abnormal transcription regulation; ii) a GIP-derived peptide; and b) administering said GIP-derived peptide to said patient under conditions such that at least one said symptom is reduced. In one embodiment, the GIP derived peptide comprises SEQ ID NO: 85.

Definitions

The terms used herein are generally intended to be interpreted according to definitions accepted within in the art, with the following exceptions:

The term “AFP” as used herein, refers to any α-fetoprotein from a variety of species. GIP, however, is derived from human α-fetoprotein, wherein GIP is represented as SEQ ID NO:2.

The term “native GIP sequence”, “wild-type GIP sequence” as used herein, refers to any portion of a GIP amino acid comprising an amino acid sequence found in a naturally occurring GIP peptide. For example, P149 comprises the full length 34 amino acid GIP sequence derived from human α-fetoprotein.

The term “α-fetoprotein-derived”, “AFP-derived” or “GIP-derived” as used herein, refers to any amino acid sequence having homology to GIP and/or P149 but is not a native GIP sequence. For example, a GIP-derived peptide or protein is a homolog of the native GIP sequence.

The term “GIP fragment” or “GIP subfragment” as used herein, refers to any peptide having at least one GIP-derived portion and/or at least one GIP native portion but is less than the entire GIP protein.

The term “consensus sequence” as used herein refers to a protein sequence which is derived by comparison of two or more protein sequences and which describes the amino acids most often present in a given segment of the protein; the consensus sequence is the canonical sequence. For example, a consensus sequence may describe any combination of identified peptide amino acid sequences that are represented by a GIP-derived homolog. Further, the term “consensus sequence” may also refer to peptide fragments and or subfragments contained within a larger “consensus sequence”.

The term “portion” when used in reference to a protein (as in a “portion of a given protein”) refers to fragments of that protein. The fragments may range in size from four (4) contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

A “variant” of a polypeptide sequence is defined as an amino acid sequence which differs by one or more amino acids from the polypeptide sequence. The variant may have “conservative” changes, wherein a substituted amino acid has similar structural or chemical properties (e.g., replacement of leucine with isoleucine). More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological or immunological activity may be found using computer programs well known in the art, for example, DNAStar software.

An amino acid sequence which is a “homolog” or has “homology” to a native or wild-type GIP sequence (e.g., SEQ ID NO: 1) is a “GIP-derived sequence” and is defined herein as an amino acid sequence which exhibits more than 50%, more preferably at least 70%, and most preferably at least 95% identity to the GIP sequence.

The term “calcium channel” as used herein refers to a peptide sequence comprising at least α₁ subunit. More preferably, a calcium channel further comprises α₂, β, γ, and δ subunits. The amino acid sequences of calcium channel subunits in different organisms (e.g., human, rabbit, and rat) and in different tissues of the same organism (e.g., neuronal tissue, cardiovascular tissue) are known in the art. Ellis et al., Science 241(4873):1661-4 (1988); Williams et al., Neuron 8(1):71-84 (1992); Ellis et al. U.S. Pat. No. 5,686,241 (hereby incorporated by reference); and Harpold et al., U.S. Pat. No. 5,792,846 (hereby incorporated by reference).

The term “protecting group” as used herein, are those groups which prevent undesirable reactions (such as proteolysis) involving unprotected functional groups. For example, the present invention contemplates protecting groups including, but not limited to, an acyl, an amide, an acetate, a benzyl, or a benzoyl group. The present invention also contemplates combinations of such protecting groups. Alternatively, a “protecting group” may comprise at least one D-amino acid.

The term “decoy receptor” as used herein, refers to a soluble form a recpetor that can act as a ligand sink. Conseqeuntly, a decoy receptor would be expected to reduce a ligand concentration to below detectable leves by a membrane-bound form of the receptor.

The term “decoy ligand” as used herein, refers to a ligand that binds to a receptor but does not activate associated celluar response cascade pathways.

The term “abnormal” as used herein, refers to any biological, physiological, psychological, biochemical, or the like, measured or observed parameter exhibited by a patient that deviates from the normal, average, or expected. For example, any quantitative measurement that is outside the normal homeostatic balance would be considered abnormal.

### BRIEF DESCRIPTION OF THE FIGURES

The following figures are not intended to be limiting to the invention and illustrate only specific embodiments of the invention contemplated herein.

FIG. 1 presents several embodiments of the GIP peptide and subfragments.
Panel A provides one embodiment of a cyclic P149c.
Panel B illustrates one embodiment of a non-solvent computer model of a cyclized amino acid sequence.
Panel C illustrates one embodiment of a full-length human α-fetoprotein comprising 590 amino acids (i.e., SEQ ID NO: 1). The GIP fragment is identified by the darkened circles (amino acid #'s 445-480).

FIG. 2 presents exemplary data for both in vivo and in vitro growth suppressive activities of the GIP-derived P149 and control peptides are depicted in GI-101 human ductal breast carcinoma and in MCF-7 human glandular breast carcinoma.
Panels A and A2: GI-101 and MCF-7 xenograft in nude mice administered P149 time-release pellets for 60 days and 30 days, respectively (N=5 mice/group).
Panels B and B2: GI-101 and MCF-7 in cell cultures treated with P149 and P187 at concentrations from 10-8 to 10-12 M for 6 days and assayed by ATP luminescence reagent employing a luciferin-luciferase system. The ATP assay, used to distinguish between cytostasis versus cytotoxicity, was used to confirm that P149 was cytostatic in its cell inhibitory activity at 6 days (see text for details). P149 and P187 were both significantly different p<,05, asterisk) than control peptides.

FIG. 3 presents exemplary data showing the growth suppressive activities of the oligomeric forms of P149, cyclic versus linear, are depicted in various in vitro and in vivo models. Biological activity was determined by three growth-dependent bioassays, namely;
Panel-A and A1) an immature rodent uterine bioassay;
Panel-B and B 1) an in vitro MCF-7 foci assay;
Panel-C and C1) an in vitro tumor cell cytostatic assessment. Results show that the linear peptide was highly dose effective in the mouse uterine assay (A1), and the MCF-7 foci-assay (B1), but not the in vitro cytostatic-assay (C1); while the cyclic configuration performed poorly in the uterine (A) and foci-assays (B), but extremely well in the in vitro cytostatic assays (C). See text for details. Scrambled peptide=P327.

FIG. 4 presents exemplary data showing a long-term administration of P149 to mice having BI-101 breast cancer.
Panel A. The P149 peptide (0.5 μg/day, released for 60 days from pellet implants) also inhibited the in vivo growth of human breast cancer GI-101 cells, a non-estrogen-dependent tamoxifen-resistant ductal carcinoma transplanted as a xenograft in nude mice. Treated animals survived 45 days after peptide depletion. After 105 days the mice were sacrificed and tumor volumes were recorded as a growth indicator.
Panel B. Mice similarly implanted with MDA-MB-231 an estrogen receptor negative cell line was similarly treated with P149. This xenograft was only inhibited in its growth by 20-30%.

FIG. 5 presents a larger figure of the inset in FIG. 4A.

FIG. 6 presents exemplary data showing the growth suppressive activity of P149 in the mouse mammary tumor isograft is demonstrated by the peptide's suppression of tumor cell proliferation and ascites fluid accumulation at a dose inoculum of 3×10⁶ tumor cells. * P<0.05; P263=control peptide; GIP=P149; GIP fragments=P149a, P149b, and P149c (see legend to FIG. 1).

FIG. 7 presents exemplary data showing the cellular adhesion of cancer cells to extra-cellular matrix (ECM) proteins and GIP for both human MCF-7 breast cancer cells and for murine 6WI-1 ascites-adapted mammary tumor cells. The cellular adhesion assay was performed in 96-well microtiter plates and the wells were pre-coated with 100 microliters of each of the above-mentioned ECM proteins for 24 hours, tumor cells added, and the plates were then incubated at 37° C. While 6WI-1 cells usually required on 2-4 hours incubation for adhesion, MCF-7 cells required 24 hours for attachment following cell trypsinization and plating. The plates were washed twice with Hank's Saline, fixed in 0.5 mL or 37% formaldehyde, stained with crystal violet and read at 570 nm on a microplate reader. Note that GIP can provide a matrix attachment surface for tumor cell adhesion and that rabbit antibodies to GIP decrease tumor cell adhesion.

FIG. 8 presents exemplary data showing the inhibition of P149 in the cellular adhesion of cancer cells to extracellular matrix (ECM) proteins for both human MCF-7 breast cancer cells and for murine 6WI-1 ascites-adapted mammary tumor cells. The cellular adhesion assay was performed in 96-well microtiter plates (Nunc Company, Denmark, catalog #263339) and the wells were pre-coated with 100 microliters of each of the above-mentioned ECM proteins for 24 hours as previously determined. After adding tumor cells, the plates were then incubated at 37° C. for various times; while 6WI-1 cells usually required only 2-4 hours incubation for adhesion; MCF-7 cells required 24 hours for attachment following trypsinization. For both cell lines, the cell culture media contained 5% calf serum to ensure cell attachment. The plates were washed twice with Hank's Saline, fixed in 0.5 mL of 37% formaldehyde, stained with crystal violet and read at 570 mm on a microplate reader. Inset: Using a CyQUANT® stain kit, (Hoechst Corp., Germany) the plates were first exposed to a freeze/thaw cycle at −70° C. for 24 hours and 37° C. for 10 minutes. CyQUANT® buffer solution (150 microliters) was added to each well, and the cell adhesion was measured by fluorescence spectroscopy and quantitated using an ImageQuant® plate scanner (Fluorimager 595, Molecular Dynamics, Sunnyvale, Calif.).

FIG. 9 presents exemplary data showing platelet aggregation modulation using 0.5 ml of normal human platelet-rich plasma (PRP) with varying amounts of P149 or P 149b using 2.5×10⁶ platelets per reaction vessel. After 2 minutes of stirring to obtain temperature equilibrium at 37° C., adenosine diphosphate (ADP) was added to the reaction vessel
Panel A: ADP incubated with P149 inhibited all phases of the platelet aggregation;
Panel B: P149b inhibited 70% of platelet aggregation;
Panel C: platelet aggregation by P149 as measured by CD62P (P-selectin) fluorescence quenching by means of flow cytometry.

FIG. 10 presents exemplary data showing platelet aggregation activation by arachidonic acid (AA) and collagen using P149-treated platelet suspensions
Panel A1: AA alone;
Panel A2: AA followed by epinephine (EPI) and EPI alone using P149b;
Panel A3: CD62P flow cytometry fluorescence quenching by P149;
Panel B1: collagen alone and collagen+P149;
Panel B2: collagen alone; collagen+P149a showing aggregation diminution.

FIG. 11 presents exemplary data using MCF-7 breast cancer cells cultured for 3-4 days (80% confluence) in order to study tumor cell attachment, migration, and spreading. The MCF-7 cells were grown to confluence and harvested using trypsin digestion. The separated cells were then resuspended in DC5. One day prior to cell seeding, glass coverslips were incubated in P149 or ovalbumin peptide concentrations extending from 0.1 mg/ml to 1000 mg/ml. MCF-7 tumor cells were seeded at 3×10⁵ cells/well and incubated for 24 hours on the coverslips. Using the Crystal Violet stain, 100 microliters of 37% formaldehyde was then added to each well for 5 minutes for cell fixation onto the plates, the wells were washed twice, and 200 microliters of Crystal Violet staining solution was added, washed and dried. The coverslips were examined under light microscopy (see text).

FIG. 12 presents an exemplary peptide having homology to the human α-fetoprotein SEQ ID NO:1. Section I presents several embodiments of extracellular matrix-associated proteins. Section II presents several embodiments of matrix metalloproteinase-associated proteins. Section III presents several embodiments of clotting and/or adhesion-associated proteins.

FIG. 13 presents exemplary data showing a time course of GIP-derived protein stimulation of mRNA expression in MCF-7 cell culture.

FIG. 14 presents exemplary data showing a dose response of linear GIP-induced stimulation of mRNA expression in MCF-7 cells.

FIG. 15 presents exemplary data showing a time course of cyclic GIP-induced stimulation of mRNA expression in MCF-7 cells.

FIG. 16 presents exemplary data showing a dose response of cyclic GIP-induced stimulation of mRNA expression in MCF-7 cells.

FIG. 17 presents exemplary data showing an extended dose response of cyclic CIP-induced stimulation of Cyclin E1 mRNA expression.

FIG. 18 presents exemplary data showing the effects of various Cyclin E's on P21, P27, p-P27 and β-actin expression.

FIG. 19 illustrates one embodiment of a voltage clamp experimental set-up.

FIG. 20 presents exemplary data showing modulation of LNCaP membrane cell current by NMDG⁺.

FIG. 21 presents exemplary data showing the effect of linear GIP on LNCaP cell membrane current.

FIG. 22 presents exemplary data showing I-V curves from LNCaP cells following exposure to scrambled, linear, and cyclic GIP embodiments.

FIG. 23 presents exemplary data showing the effects of one embodiment of a GIP-derived protein on gamma radiation and dexamethasone induced apoptosis.

### DETAILED DESCRIPTION OF THE INVENTION

The present invention is related to an α-fetoprotein (AFP) derived peptide termed the Growth Inhibitory Peptide (GIP). In one embodiment, GIP comprises consensus amino acid sequences modulating a variety of biological activities. In one embodiment, the modulated biological activities include, but are not limited to, anti-cancer effects, nonselective calcium channel regulation, angiogenesis inhibition, cytoskeletal controls, cell cycle regulation, enzyme function, transcription, and anti-microbials.

I. α-Fetoprotein-Derived Growth Inhibitory Proteins

Human mammalian α-fetoprotein (AFP) is a known tumor-associated fetal protein synthesized mostly in fetal liver and hepatomas. Mizejewski G, “New insights into AFP structure and function: Potential biomedical applications” In: Alpha-fetoprotein and Congenital Disorders, Eds. Mizejewski G, Porter I H. 1985, Orlando: Academic Press. pp. 5-34 (1985); and Mizejewski G, “Alpha-fetoprotein structure and function: relevance to isoforms, epitopes, and conformational variants” Exp Biol Med 226: 377-408 (2001). The human fetal protein has a generally α-helix tertiary structure comprising three domains of nearly 200 amino acid each, and has a molecular mass of approximately 69,000 Daltons. Interestingly, the growth suppressive GIP fragment is closed within the helix (i.e., encrypted) and is not accessible to binding agents. Mizejewski et al., “Alpha-fetoprotein derived synthetic peptides: assay of an estrogen-modifying regulatory segment” Mol Cell Endocrinol 118:15-23 (1996). Although the GIP fragment has known anticancer activity, the native full-length AFP protein displays growth enhancing properties regardless of whether the tissue is fetal or cancer in origin. Gershwin et al., “Accelerated plasmacytoma formation in mice treated with alpha-fetoprotein” J Natl Cancer Inst 64:145-149 (1980); and Li et al., “The intracellular mechanism of alpha-fetoprotein promoting the proliferation of NIH-3T3 cells” Cell Res 12:151-156 (2002).

It has previously been reported that a 34-mer β-sheet GIP-derived peptide segment (i.e., for example, P149 or SEQ ID NO:2) in the third domain of AFP can transiently bestow a growth suppressive property upon the full-length AFP molecule, in contrast to its growth enhancing properties. Mizejewski et al., “Alpha-fetoprotein derived synthetic peptides: assay of an estrogen-modifying regulatory segment” Mol Cell Endocrinol 118:15-23 (1996). A synthetic-peptide isolated from this third domain of AFP encompasses a sequence stretch of 34 amino acids which lie hidden in a molecular cleft in the native AFP molecular form. See FIG. 1B. This epitope cannot be detected using present-day, commercially available, antisera to AFP and requires a separate antibody, distinct from monoclonal antibodies to AFP. Vakharia et al., “Human alpha-fetoprotein peptides bind estrogen receptor and estradiol, and suppress breast cancer” Breast Cancer Res Treat 63:41-52 (2000); Sinenko et al., “Analysis of functional activities of human alpha-fetoprotein with new monoclonal antibodies and a synthetic peptide” Tumor Biol 21:112 (2000). This hidden (i.e., encrypted) peptide segment was initially discovered by sequence similarity with members of the heat shock protein family, which are associated with: 1) steroid receptor binding; and 2) transient protein folding in the endoplasmic reticulum. Although it is not necessary to understand the mechanism of an invention, it is believed that exposure of the GIP-derived 34-mer segment probably represents a folding intermediate form of AFP. Mizejewski G, “Alpha-fetoprotein structure and function: relevance to isoforms, epitopes, and conformational variants” Exp Biol Med 226: 377-408 (2001). Indeed, this exposed peptide segment meets the described qualifications of a folding intermediate marker of the native AFP molecule. Kuznetsova et al., “Use of the phase diagram method to analyze the protein unfolding-refolding reactions: fishing out the “invisible” intermediates” J Proteome Res 3:485-494 (2004).

All peptides described herein may be synthesized by F-MOC chemistry using an Applied BioSystems 431A peptide synthesizer (Foster City, Calif.) as previously described and purified on reverse phase high pressure liquid chromatography (HPLC). Eisele et al., “Studies on a growth-inhibitory peptide derived from alpha-fetoprotein and some analogs” J Pept Res 57:29-38 (2001); Eisele et al., “Studies on analogs of a peptide derived from alpha-fetoprotein having anti growth properties” J Pept Res 57:539-546 (2001); MacColl et al., “Interrelationships among biological activity, disulfide bonds, secondary structure, and metal ion binding for a chemically synthesized 34-amino-acid peptide derived from alpha-fetoprotein” Biochim Biophys Acta 1528:127-134 (2001); and Butterstein et al., “Biophysical studies and anti-growth activities of a peptide, a certain analog and a fragment peptide derived from alpha-fetoprotein” J Pept Res 61:213-218 (2003).

Synthesized control peptides include, but are not limited to, an albumin-derived peptide (P263) containing homologous amino acid sequences to GIP and a scrambled P149 peptide (P327). Peptide subfragments of P149 were also synthesized representing an amino terminal segment (P149a, comprising 12 amino acids), a middle segment (P149b, comprising 14 amino acids), and a carboxy terminal segment (P149c, comprising 8 amino acids). Finally, GIP amino acid numbers 4 & 15 (i.e., SEQ ID NO:1 amino acid positions 448 & 459, respectively) may contain an aspartic acid to aspargine (D→N) mutated peptide (P187) was employed to study the cytostatic action of GIP in comparative experiments.

The biochemical/biophysical studies of P149 identified a peptide with a molecular mass of 3573 Daltons as determined by electrospray ionization mass spectroscopy. Eisele et al., “Studies on a growth-inhibitory peptide derived from alpha-fetoprotein and some analogs” J Pept Res 57:29-38 (2001); MacColl et al., “Interrelationships among biological activity, disulfide bonds, secondary structure, and metal ion binding for a chemically synthesized 34-amino-acid peptide derived from alpha-fetoprotein” Biochim Biophys Acta 1528:127-134 (2001); and Butterstein et al., “Effect of alpha-fetoprotein and derived peptides on insulin- and estrogen-induced fetotoxicity” Fetal Diagn Ther 18:360-369 (2003). The far-UV circular dichrosim (CD) displayed a negative maximum at about 201 nanometers and indicated the presence of β-sheets/turns (45%) and other ordered structures in (45%) equal proportions, with the remaining structures composed of α-helix (10%) forms. MacColl et al., “Interrelationships among biological activity, disulfide bonds, secondary structure, and metal ion binding for a chemically synthesized 34-amino-acid peptide derived from alpha-fetoprotein” Biochim Biophys Acta 1528:127-134 (2001); Butterstein et al., “Biophysical studies and anti-growth activities of a peptide, a certain analog and a fragment peptide derived from alpha-fetoprotein” J Pept Res 61:213-218 (2003); and Butterstein et al., “Effect of alpha-fetoprotein and derived peptides on insulin- and estrogen-induced fetotoxicity” Fetal Diagn Ther 18:360-369 (2003). Both Fourier-infrared spectroscopy and GCG computer modeling software further confined the presence of a largely β-sheet structure for the peptide. A cyclic version of P149 was synthesized together with the single-letter amino acid code sequences of GIP, its fragments, and control peptides.

One embodiment of a full length human α-fetoprotein comprises 590 amino acids (SEQ ID NO:1). Mezijewski et al., “a-Fetoprotein growth inhibitory peptides: Potential leads for cancer therapeutics” Mol Cancer Therap 2:1243-1255 (2003). See FIG. 1C. The AFP protein is believed to comprise three domains in a U-shaped configuration. It can be seen that these domains are generated by several disulfide bridges from cysteine residues. Various embodiments are contemplated by the present invention comprising GIP and GIP-derived peptides defined by various combinations of α-fetoprotein amino acid positions ranging between 445-478, relative to FIG. 1C. For example, GIP, also refered herein as P149 (SEQ ID NO:2) represents a wild type GIP-derived amino acid sequence. The present invention also contemplates a mutant GIP-derived amino acid sequence also representing α-fetoprotein amino acid positions 445-478, but where C⁴⁵⁵ & C⁵⁶⁸ may become any amino acid (X) (SEQ ID NO:9).

II. GIP Subfragments by Homology Comparison

Some embodiments of the present invention contemplate GIP-derived subfragments that were compared with protein sequences derived from the Genebank databases (for example, see FIG. 12) using the GCG (Wisconsin Program) FASTA sequence comparison software as described. Dauphinee et al., “Human alpha-fetoprotein contains potential heterodimerization motifs capable of interaction with nuclear receptors and transcription/growth factors” Med Hypotheses 58:453-461 (2002).; and Mizejewski G, “An apparent dimerization motif in the third domain of alpha-fetoprotein: molecular mimicry of the steroid/thyroid nuclear receptor superfamily” Bioessays 15:427-432 (1993). Amino acid sequence homology to GIP-derived peptides was found with a variety of proteins associated with a wide variety of biological activities.

A. Anti-Cancer GIP Homologs

In some embodiments, GIP peptides are homologous with sequences found in peptides having anti-cancer activity including, but not limited to, integrins, the extracellular matrix-associated proteins (ECM proteins), metalloproteinase-association proteins, clotting factors and/or adhesion-associated proteins. (for example, see FIG. 12)

| In some embodiments, anti-tumor GIP-derived wild type subfragments comprise: | | |
|---|---|---|
| 445-480 (linear): | LSEDKLLACGEGAADIIIGHLCIRHEMTPVNPGVGQ | (36-mer; SEQ ID NO:141) |
| 445-478 (linear): | LSEDKLLACGEGAADIIIGHLCIRHEMTPVNPGV | (P149; SEQ ID NO:2) |
| 445-456 (linear): | LSEDKLLACGEG | (P149a; SEQ ID NO:3) |
| 457-470: (linear) | AADIIIGHLCIRHE | (P149b; SEQ ID NO:4) |
| 470-478 (linear) | MTPVNPGV | (P149c; SEQ ID NO:5) |
| 445-478 (linear): | LSENKLLACGEGAANIIIGHLCIRHEMTPVNPGV | (P187; SEQ ID NO:142) |
| 477-478 (linear): | HEMTPVNPGV | (SEQ ID NO:6) |
| 449-467 (linear): | LLACGEGAADIIIGHLCIR | (SEQ ID NO:7) |
| 464-449 (linear): | LSEDK | (SEQ ID NO:8) |
| In some embodiments, GIP C⁴⁵⁵ & C⁵⁶⁸ mutant subfragments comprise: | | |
| 445-478 (linear): | LSEDKLLAXGEGAADIIIGHLXIRHEMTPVNPGV | (SEQ ID NO:9) |
| 445-456 (linear): | LSEDKLLAXGEG | (SEQ ID NO:10) |
| 449-467 (linear): | LLAXGEGAADIIIGHLXIR | (SEQ ID NO:11) |
| 457-470 (linear): | AADIIIGHLXIRHE | (SEQ ID NO:12) |

wherein X may be any amino acid.

Various control peptides relevant to GIP-derived peptide anti-cancer activity comprise:

P192:

(SEQ ID NO:13)

KDFIHKQGVALQTMKQELINLVKELVKPQIEAVADFSGLL
P263:

(SEQ ID NO:14)

HPEAKRMPCAEDYLSVVLNQLCVLEHKTPVSDRV
P327:

(SEQ ID NO:15)

GINHLPSDAVGTAIEVMPCLRDGKLCIGAEHIEL

GIP is a major participant in tumor cell surface interactions. GIP is a potent suppressor of tumor proliferation in both rodent and human cell models. GIP's biological activity is dependent on its oligomeric state, specifically its trimer (linear) versus monomer (cyclic) configuration. Biological activity was determined by three growth-dependent bioassays, namely, i) an immature rodent uterine assay; ii) an in vitro MCF-7 foci assay; and iii) an in vitro cytostatic assessment. Results indicated that the linear peptides was highly dose effect in the mouse uterine assay (49%), the MCF-7 foci-assay (65-70%), but not in the in vitro cytostatic assay. Conversely, the cyclic configuration performed poorly in the uterine and MCF-7 foci-assays (20-25%) but performed extremely well in the in vitro cytostatic assays (80-90%). The cyclic form in the uterine and foci assay was more susceptible to proteolytic serum degradation (5-10% serum) and/or the cyclic form in vivo presents and altered binding interface for cell surface receptor dimerization and clustering.

It is known that α-fetoprotein may be taken up and accumulated in the cytosol by various types of tumor and lymphoid cells after binding to the cell surface. For example, photomicrographs of P149 uptake into MCF-7 breast cancer cells showed binding of P149 to the cell surface in a granular fashion within five minutes of exposure. This was followed by diffusion of P149 throughout the cytoplasm within one hour. Fluorescent antibody localized P149 condensed around the nuclear membrane but remained cytoplasmic. Mizejewski G., “Biological role of α-fetoprotein in cancer; prospects for anticancer therapy” Exp Rev Anticancer Ther 2:709-735 (2002).

Cellular activities such as cell adhesion, migration, aggregation, agglutination, cytoskeletal influence on cell shape and form, and endocytosis indicate that GIP is interactive with the cell plasma membrane and invokes a cascade of physiological responses initiated by integrin-associated and disintegrin-like therapeutic actions with tumor growth, progression, and metastasis.

It has been demonstrated that the GIP was capable of growth suppression in a multitude of human tumors both in vitro and in vivo. It was proposed that the GIP may exert its anti-proliferative effect by serving as a decoy ligand for G-coupled growth factor receptors. Although it is not necessary to understand the mechanism of an invention, it is believed that this mechanism of growth inhibition by GIP might be linked to a G-protein MAP kinase uncoupling mechanism in a downstream step of signal-transduction. Mizejewski et al., “Alpha-fetoprotein growth inhibitory peptides: potential leads for cancer therapeutics” Mol Cancer Ther 2:1243-1255 (2003).

It is known that GIP can mediate growth suppression in both fetal and tumor cells, but not in adult differentiated cells. Although it is not necessary to understand the mechanism of an invention, it is believed that GIP is highly interactive at the plasma membrane interface in biological activities such as endocytosis, cellular agglutination, and cytoskeletal-induced/cell shape and form changes. It is also known that GIP mediates growth suppression in nine different human tumor types including, but not limited to, breast, prostate, and ovary cancer and to suppress the spread of tumor infiltrates and metastases in human and mouse mammary cancer.

In one embodiment, a GIP-derived peptide and/or subfragments inhibits tumor cell adhesion to extracellular matrix-associated proteins. In another embodiment, a GIP-derived peptide, and/or subfragments blocks platelet aggregation. In one embodiment, an oligomeric GIP-derived peptide is more efficacious than a monomer GIP-derived peptide. In one embodiment, an oligomeric GIP-derived peptide is selected from the group comprising a dimer, a trimer, a tetramer, or a hexamer. Although it is not necessary to understand the mechanism of an invention, it is believed that inhibiting tumor cell adhesion and blocking platelet aggregation prevents tumor cell spreading and/or migration that can lead to metastatic infiltration into host tissues (i.e., for example, lung and pancreas). It is further believed that GIP may be related to a variety of alpha/beta chain integrin proteins because Genebank amino acid sequence comparisons have identified homologous sequences.

Recent findings also indicate that AFP can stimulate the expression of certain oncogenes (c-fos, c-jun, and n-Ras) to enhance the proliferation of human hepatocellular carcinoma cells. Human AFP, in vitro, has recently been shown to functionally impair dendritic cells inducing dysfunction and apoptosis of antigen processing cells (APCs).

Some of the most potent growth inhibitors are peptide fragments derived from plasma or extracellular matrix-associated (ECM) proteins whose full-length sequences do not inhibit growth. These plasma and/or extracellular matrix proteins have been commonly identified as storage depots for growth modulatory peptide segments in the field of signal transduction and growth regulation. Likewise, it has been reported that full length AFP (at physiologic and/or pharmacological dose levels) may enhance tumor growth.

For tumor cells to metastasize they are believed to enter the blood stream and disperse. Although it is not necessary to understand the mechanism of an invention, it is believed that tumor cells loosen the tight junctions holding them together, detach from their basement membrane, and change their shape so that they may slip through breaks in the membrane generated by local metalloproteinases. It is further believed that cell shape changes and plasma flow assist tumor cells to pass between endothelial cells and enter tissue capillaries. The plasma membrane of the metastasized tumor cells are believed to contain proteins that may enhance their settling and attachment to the microenvironment of distant sites.

FIG. 12 presents exemplary human proteins that have sites with high homology to GIP, and which may therefore be targets for functional interference by GIP during the metastasis process.

1. Cancer Cell Culture Studies

The first report of the anticancer effect of P149 was observed in an estrogen-dependent contact-inhibition in vitro assay employing the human breast cancer MCF-7 cell line. Mizejewski et al., “Alpha-fetoprotein derived synthetic peptides: assay of an estrogen-modifying regulatory segment” Mol Cell Endocrinol 118:15-23 (1996). The P149 peptide inhibited the formation of tumor cell foci formed as a result of the accumulation of breast cancer cells which clump together due to loss of contact inhibition. See Table 1.

TABLE 1
GIP Protein Fragment Biological Activities Both In Vivo And In Vitro

| Peptide Segment/Cell | | % Inhibition Of Activity | | | | |
|---|---|---|---|---|---|---|
| Activity | P149 | P149a | P149b | P149c | P263 | P192 |
| 1. 6WI-1 | | | | | | |
| A. Body Weight | 48 | 22 | 31 | 44 | 13 | 0 |
| B. Cell Prol | 50 | 28 | 34 | 45 | 5 | 0 |
| C. Acites Volume | 42 | 21 | 26 | 39 | 10 | 0 |
| D. Pup Ascites | 56 | 21 | 32 | 29 | 0 | 0 |
| | (50) | | | | | |
| E. Peptide Combos | NA | P149a, | 44 | | 0 | 0 |
| | | b | | | | |
| 2. Kidney tumor | 70 | 25 | 50 | 19 | 0 | 0 |
| growth | | | | | | |
| 3. MCF-7 foci | 68 | 33 | 31 | 45 | 0 | 0 |
| formation | (20) | | | | | |
| 4. Platelet Agg. | | | | | | |
| A) ADP | 90 | 40 | 95 | 0 | 0 | 0 |
| B) AA | 100 | 0 | 100 | 0 | 0 | 0 |
| C) Coll | 90 | 0 | 90 | 0 | 0 | 0 |
| 5. Uterine Growth | 39 | 28 | 22 | 42 | 0 | 0 |
| A. Enzymatic | NA | 24 | 19 | 44 | 0 | 0 |
| Fragments | | | | | | |
| 6. Cell Adhesion | 65 | ND | 58 | 56 | 0 | 0 |
| | | | (C) | | | |
| 7. Estrogen | 73 | 63 | 67 | 37 | 0 | 0 |
| Fetotoxicty | | | | | | |

The estrogen-induced foci suppression by GIP was extremely potent, displaying peak inhibitory doses at 10⁻¹⁰ to 10⁻¹² M concentrations. Following that initial study, summary findings by the National Cancer Institute's “Therapeutics Drug Screening Program” (Bethesda, Md.) concerning the GIP were reported. See Table 2.

TABLE 2
GIP-Induced (P149) Growth Suppression NCI Screening Results*

| | | | 2-Day Assay | | 6-Day Assay | |
|---|---|---|---|---|---|---|
| Tissue Of | | Tumor | | % | | % |
| Origin Human- | Cell Line | Tissue | Conc. | Growth | Conc. | Growth |
| Derived | Designation | Type | (Molar) | Inhibition | (Molar) | Inhibition |
| 1. Colon | KM-12 | AC | 10⁻², 10⁻¹ | 10-20 | 10⁻⁵-10⁻⁷ | 75 |
| | HCC-299 | AC | 10⁻¹ | 20 | 10⁻⁵-10⁻⁷ | 80 |
| | Colo-205 | AC | 10⁻² | 10 | 10⁻⁵ | 10 |
| | HCT-116 | AC | 10⁻², 10⁻¹ | 15 | 10⁻⁵-10⁻⁷ | 75 |
| 2. Ovary | OVCAR-3 | AC | 10⁻² | 20 | 10⁻⁵-10⁻⁷ | 80 |
| | SK-OV-3 | AC | 10⁻², 10⁻¹ | 10 | 10⁻⁵-10⁻⁷ | 60 |
| | IGROV1 | AC | 10⁻¹ | 10 | 10⁻⁵-10⁻⁷ | 75 |
| | OVCAR-4 | AC | 10⁻², 10⁻¹ | 20 | 10⁻⁵-10⁻⁷ | 85 |
| 3. Breast | MCF-7 | AC | 10⁻² | 20 | 10⁻⁵-10⁻⁷ | 80 |
| | MDA-MB-231 | AC | 10⁻², 10⁻¹ | 25 | 10⁻⁷ only | 80 |
| | MDA-MB-435 | AC | 10⁻¹ | 20 | 10⁻⁵-10⁻⁷ | 70 |
| | BT-549 | AC | 10⁻², 10⁻¹ | 15 | 10⁻⁶-10⁻⁷ | 25-40 |
| | T-47D | AC | 10⁻² | 10 | 10⁻⁵ | 25 |
| 4. Prostate | PC-3 | AC | 10⁻¹ | 5 | 10⁻⁶-10⁻⁷ | 80 |
| | DU-145 | AC | 10⁻¹ | 10 | 10⁻⁵-10⁻⁷ | 90 |
| 5. Non-Small | HOP-62 | CA | 10⁻², 10⁻¹ | 30 | 10⁻⁵-10⁻⁷ | 75 |
| Cell Lung | NCI-H226 | CA | 10⁻² | 5 | 10⁻⁵ | 5-10 |
| | NCI-H460 | CA | 10⁻² | 30 | 10⁻⁵-10⁻⁷ | 80 |
| 6. Melanoma | UACC-62 | Epithelial | 10⁻², 10⁻¹ | 15-20 | 10⁻⁵-10⁻⁷ | 80 |
| | SK-MeL-5 | Squamous | 10⁻² | 5 | 10⁻⁵ | 10 |
| | SK-MeL-2 | Squamous | 10⁻², 10⁻¹ | 20 | 10⁻⁵-10⁻⁷ | 50-75 |
| | UACC-257 | Squamous | 10⁻² | 20 | 10⁻⁵-10⁻⁷ | 75-80 |
| 7. Central | SF-295 | CA | 10⁻², 10⁻¹ | 20-25 | 10⁻⁵-10⁻⁷ | 80 |
| Nervous | SF-539 | CA | 10⁻² | 10 | 10⁻⁵ | 15-20 |
| System | U-251 | CA | 10⁻² | 15 | 10⁻⁶-10⁻⁷ | 45 |
| | SNB-75 | CA | 10⁻², 10⁻¹ | 10 | 10⁻⁶-10⁻⁷ | 50 |
| 8. Kidney | TK-10 | Renal CA | 10⁻², 10⁻¹ | 20 | 10⁻⁴-10⁻⁷ | 85 |
| | RXF-393 | Renal CA | 10⁻¹ | 30 | 10⁻⁶-10⁻⁷ | 45-50 |
| | ACHN | Renal CA | 10⁻² | 20 | 10⁻⁷-10⁻⁷ | 80 |
| | CAK-1 | Renal CA | 10⁻², 10⁻¹ | 15 | 10⁻⁵-10⁻⁷ | 50-75 |
| 9. White Blood | K-562 | Leukemia | 10⁻², 10⁻¹ | 5 | 10⁻⁷ | 45 |
| Cell | Molt-4 | Leukemia | 10⁻² | 5 | NA | 10-15 |
| | CCRF-CEM | Leukemia | 10⁻² | 5 | 10⁻⁵ | 5-10 |

*National Cancer Institute Therapeutics Screening Program, Bethesda MD.
AC = adenocarcinoma;
CA = carcinoma.

These findings detailed the in vitro results of the GIP challenges against 60 different cell culture lines representing a variety of human cancers. Mizejewski et al., “Alpha-fetoprotein growth inhibitory peptides: potential leads for cancer therapeutics” Mol Cancer Ther 2:1243-1255 (2003).

Using a 2 day versus 6-day comparative proliferation assay employing sulforhodamine staining, the P149 peptide appeared to be cytostatic (not cytotoxic) against 38 of the 60 cancer cell lines, representing nine different cancer cell types which included prostate, breast, and ovarian cancers. In subsequent reports, the effective use of the GIP and its fragments against various breast cancers were described both in vivo and in vitro. Vakharia et al., “Human alpha-fetoprotein peptides bind estrogen receptor and estradiol, and suppress breast cancer” Breast Cancer Res Treat 63:41-52 (2000). See FIG. 2.

The P149 peptide (0.5 μg/day; 60 day time release pellet) also inhibited the in vivo growth of human breast cancer GI-101 cells using a non-estrogen-dependent tamoxifen-resistant ductal carcinoma transplanted as a xenograft in nude mice. Hurst et al., “A novel model of a metastatic human breast tumour xenograft line” Br J Cancer 68:274-276 (1993); and Morrissey et al., “A metastatic breast tumor cell line, GI-101A, is estrogen receptor positive and responsive to estrogen but resistant to tamoxifen” Cell Biol Int 22:413-419 (1998).

Similarly, using a 30-day time-release pellet, GIP suppressed the in vivo growth of estrogen-dependent growth of MCF-7 human breast cancer xenografts in nude mice using a pellet-release rate of 0.25 μg peptide/day. In accompanying cell culture cytostatic assays, the GIP suppressed, by 50-80%, the growth of four of five human breast cancer cell lines maintained in non-estrogen-supplemented cell culture media, demonstrating an estrogen-independent mode of growth inhibition. Vakharia et al., “Human alpha-fetoprotein peptides bind estrogen receptor and estradiol, and suppress breast cancer” Breast Cancer Res Treat 63:41-52 (2000); and Mizejewski et al., “Alpha-fetoprotein growth inhibitory peptides: potential leads for cancer therapeutics” Mol Cancer Ther 2:1243-1255 (2003). See FIG. 2

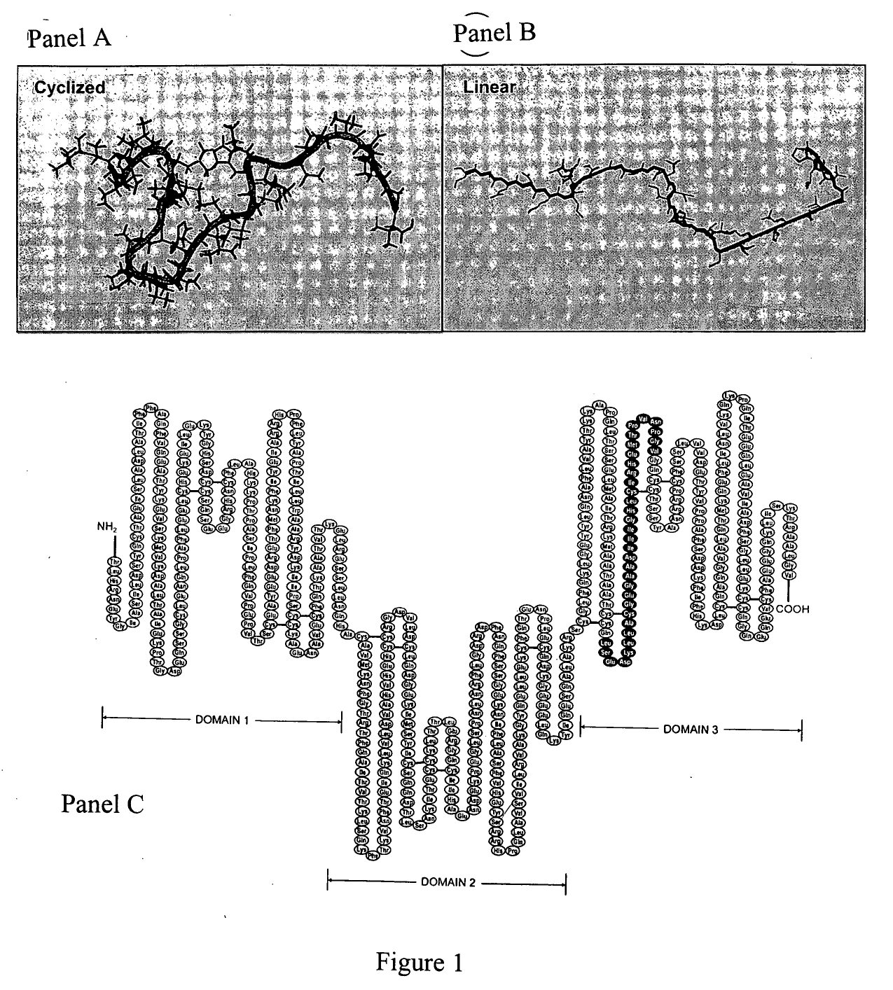

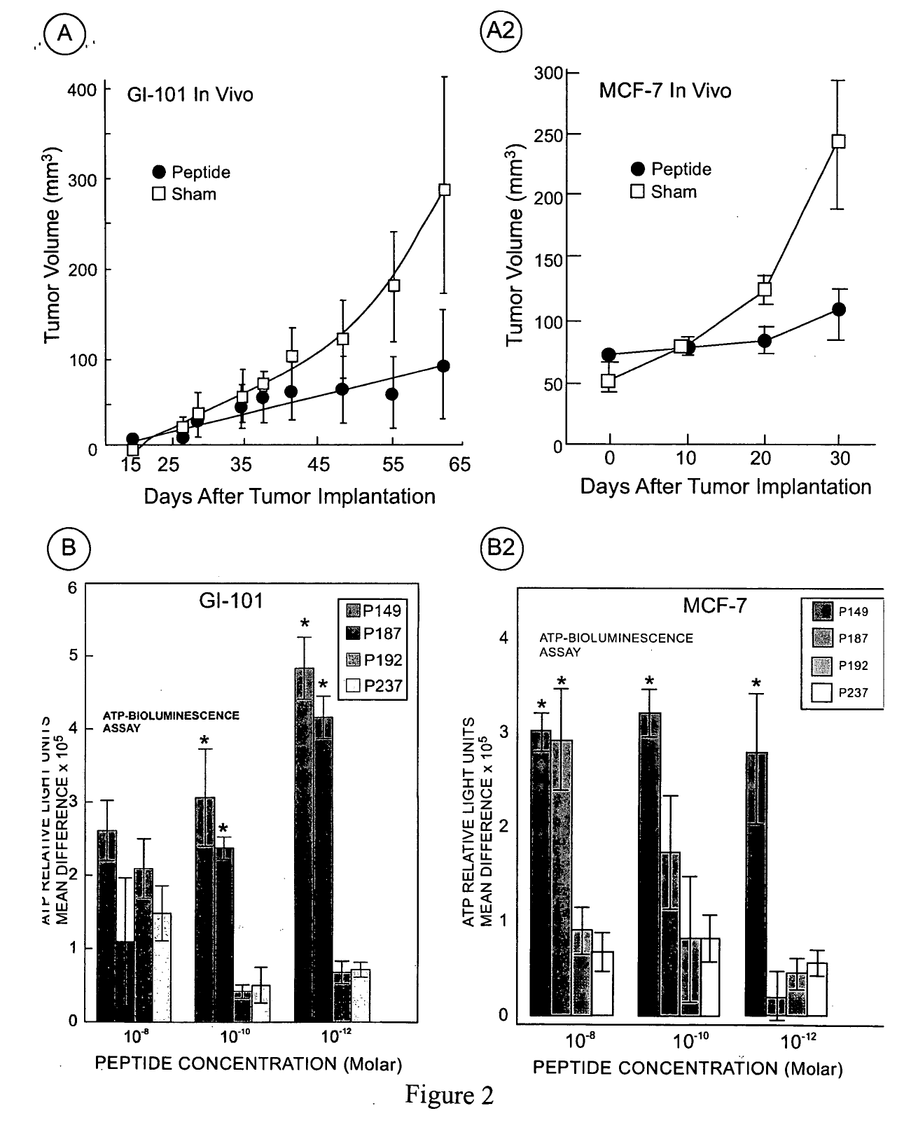

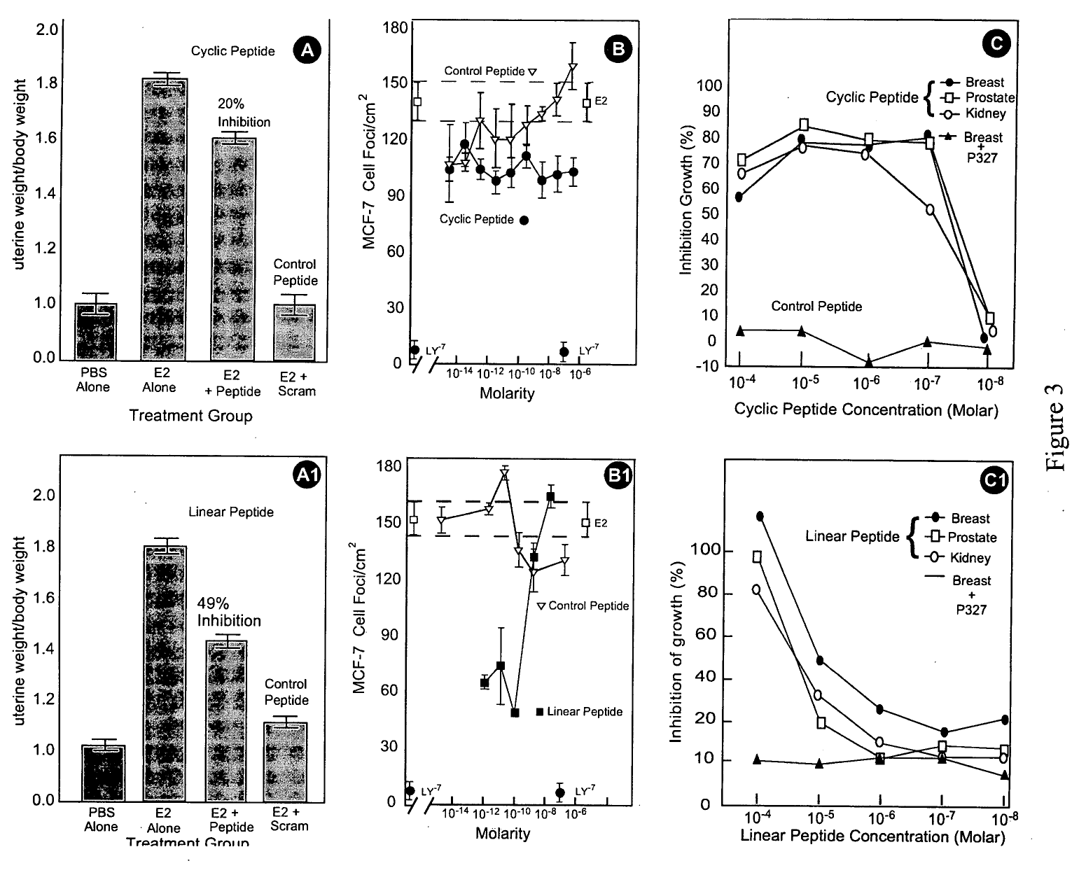

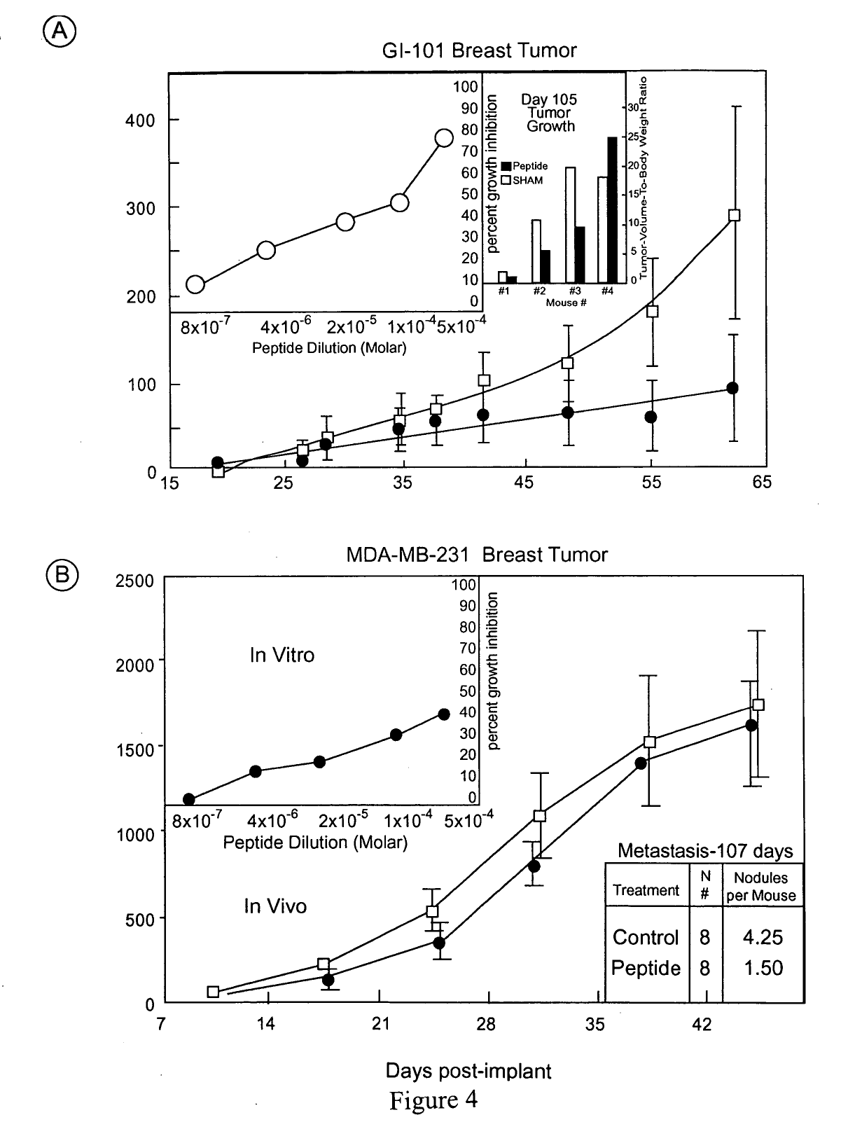

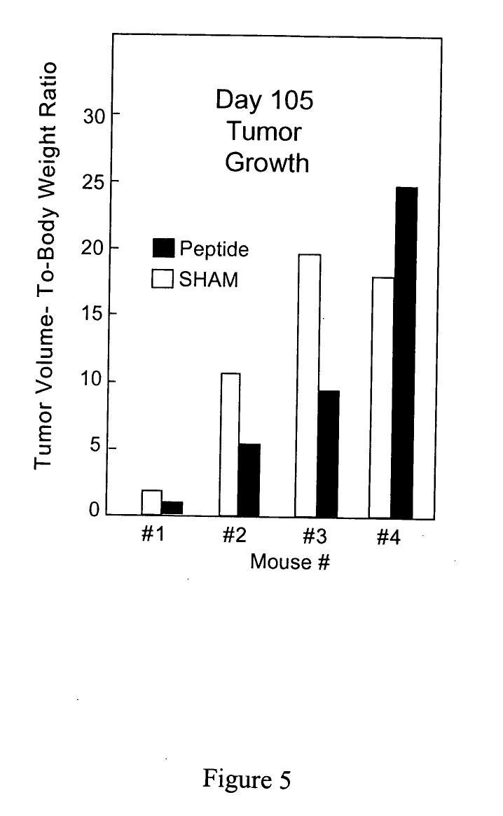

In the in vivo nude mouse xenograft studies, GIP was effective as a growth suppressor agent (70% inhibition) in the GI-101 breast tumor in vivo model even after withdrawal of peptide treatment. The GI-101 treated animals which had received the peptide pellet implant of P149 for 60 days, survived 45 days after peptide depletion. After 105 days, the mice were sacrificed and tumor volumes were recorded as a growth indicator. See FIGS. 4A and 5. When the remaining mice on GIP treatment were compared to sham controls, 3 of 4 surviving animals continued to display 40-50% tumor growth suppression.

Nude mice were also implanted with MDA-MB-231 (Non-ER) breast tumor cells, and similarly treated with P149 implants. These tumors showed only minor growth suppression (20%-30%) in vivo during use of the 60-day GIP implants similar to the sulforhodamine in vitro assays which exhibited 30-40% growth suppression. See FIG. 4B. However, histopathological analysis following autopsy on day 105 (i.e., for example, after GIP depletion) revealed that the tumor-bearing nude mice that received the GIP implants displayed lung metastases on an average of 1.5 nodules per lobe compared to 4.25 nodules/lobe in the control tumor-bearing, mice (5 mice/group).

In order to confirm whether P149 was indeed cytostatic, and not cytotoxic, an ATP-based cytostatic assay was performed which measures the decrease in cytosolic ATP during drug (i.e., for example, GIP peptide) treatment. This ATP luminescence assay has long been employed to distinguish between cytostatic versus cytotoxic chemotherapeutic sensitivity of drugs. Garewal et al., “ATP assay: ability to distinguish cytostatic from cytocidal anticancer drug effects” J Natl Cancer Inst 77:1039-1045 (1986); Kuzmits et al., “The use of bioluminescence to evaluate the influence of chemotherapeutic drugs on ATP-levels of malignant cell lines” J Clin Chem Clin Biochem 24:293-298 (1986); and Kurbacher et al., “Use of an ex vivo ATP luminescence assay to direct chemotherapy for recurrent ovarian cancer” Anticancer Drugs 9:51-57 (1998). After incubation of the cell cultures for 6 days, cytosolic ATP was extracted, stabilized, and quantitated from a standard curve produced from Berthold LB-96P luminometer readings using a luciferin-luciferase counting reagent. The ATP-bioluminescence assay employing both MCF-cells and GI-101 cells exhibited a cytosolic decrease of ATP in GIP-treated mice compared to both control peptides and non-peptide treated control cells. Data displaying the decrease in cytosolic ATP over a 6-day period was deemed consistent with the presence of a cytostatic (not cytotoxic) mode of growth inhibition at peptide doses of 10⁻¹² to 10⁻⁸M. See FIGS. 2B and B2.

A mutated form of GIP (P187) employing a Asp→Asn amino acid substitutions at GIP amino acid positions 4 & 15 also indicated that the altered peptide was equally responsive as P149 at 10⁻⁸ M but lost gradually lost potency from 10⁻⁸ to 10⁻¹²M. These data are comparable to results in which P187 showed less mouse mammary tumor growth suppression than did the original P149 peptide. The GIP-derived control peptide P192, and the scrambled peptide P237, were not significantly different from the non-treated control.

The oligomeric form of GIP employed in the cytostatic assays also showed dramatic differences regarding dose effect. The cyclic form of GIP constitutes a disulfide-bridged monomer in solution, whereas GIP in the linear configuration is known to exist as an oligomeric trimer. Eisele et al., “Studies on a growth-inhibitory peptide derived from alpha-fetoprotein and some analogs” J Pept Res 57:29-38 (2001); Eisele et al., “Studies on analogs of a peptide derived from alpha-fetoprotein having anti growth properties” J Pept Res 57:539-546 (2001); and MacColl et al., “Interrelationships among biological activity, disulfide bonds, secondary structure, and metal ion binding for a chemically synthesized 34-amino-acid peptide derived from alpha-fetoprotein” Biochim Biophys Acta 1528:127-134 (2001). Using three different human cancer cell lines (breast, prostate, or kidney) it was readily demonstrable that the monomeric cyclic form of the peptide produced 80-90% growth inhibition over a range of 10⁻⁵ M to 10⁻⁷ M when the sulforhodamine cytostatic 6-day assay was employed. See FIG. 3C. In comparison, the same three cancer cell lines produced growth inhibition only at 10⁻⁴ to 10⁻⁵ M, which rapidly declined thereafter when the linear peptide was utilized.

The linear peptide is known to be in the trimer/hexamer configuration and even at a higher polymer state after prolonged storage of PBS-solubilized peptide. Eisele et al., “Studies on a growth-inhibitory peptide derived from alpha-fetoprotein and some analogs” J Pept Res 57:29-38 (2001). It is also apparent that the cyclic versus the linear version of P149 produced different dose responses when applied to the E2-dependent MCF-7 cell foci assay showing an inhibitory effect even with low concentrations of linear peptide. See FIG. 3B. E2-activated immature mouse uterine growth assay showed that the cyclic peptide was only marginally inhibitory, while the linear peptide achieved nearly 50% inhibition. Mizejewski et al., “Alpha-fetoprotein growth inhibitory peptides: potential leads for cancer therapeutics” Mol Cancer Ther 2:1243-1255 (2003); See FIG. 3A. It is apparent from these data that the oligomeric form of the GIP is crucial in determining the growth inhibitory potency of these assays in a dose-dependent fashion.

In a study employing a human kidney tumor cell line, it was also demonstrated that linear P149 and its linear subcomponent fragments possessed differing growth inhibitory properties. For example, while P149 displayed nearly 80% growth inhibition of this tumor at 5×10⁻⁴ M, fragments P232, P228, and P211 showed decreasing potencies of 50%, 25%, and 20%, respectively. As in previous animal models, the individual peptide subfragments of GIP-derived peptides largely displayed less biological activity than P149. Mizejewski et al., “Alpha-fetoprotein growth inhibitory peptides: potential leads for cancer therapeutics” Mol Cancer Ther 2:1243-1255 (2003); and Mizejewski et al., “Review and Proposed Action of alpha-fetoprotein growth inhibiting peptides as estrogen and cytoskeletal-associated factors” Intl Journal Cell Biology (In Press, 2004).

2. Tumor Cell Spreading and Migration

Tumor cell-induced platelet aggregation (TCIPA) plays a role in tumor metastasis. Tumor cells in the vasculature are frequently observed in complexes with platelets and this association, together with the hypercoagulable state of malignant disease, appears to play a role in successful metastasis. Gasic et al., “Platelet-tumor-cell interactions in mice. The role of platelets in the spread of malignant disease” Int J Cancer 11:704-718 (1973); and Gould et al., “Disintegrins: a family of integrin inhibitory proteins from viper venoms” Proc Soc Exp Biol Med 195:168-171 (1990). The ability of tumor cells to induce platelet aggregation is widespread among cancers including, but not limited to, breast carcinoma, colon adenocarcinoma, lung carcinoma, melanomas, and others. Sheu et al., “Triflavin, an Arg-Gly-Asp-containing peptide, inhibits the adhesion of tumor cells to matrix proteins via binding to multiple integrin receptors expressed on human hepatoma cells” Proc Soc Exp Biol Med 213:71-79 (1996). Platelet participation in the metastatic process is thought to result from: a) direct binding of platelets to tumor cells; and b) the release of soluble inducer agents from the tumor cells. These agents would include the classical platelet aggregation activators including, but not limited to, ADP, cathepsin B, thrombin-like proteinases, collagen, or tissue factor-generated thrombin. Sheu et al., “Interaction of thrombin-activated platelets with extracellular matrices (fibronectin and vitronectin): comparison of the activity of Arg-Gly-Asp-containing venom peptides and monoclonal antibodies against glycoprotein IIb/IIIa complex” J Pharm Pharmacol 49:78-84 (1997). Thus, platelets may act to facilitate all the intermediate steps of transvascular metastasis including tumor cell retention and arrest, subendothelial interaction, and extravasation from the microvasculature. Blockage at these platelet events might retard or reduce tumor cell metastasis. As noted above, GIP was capable of disrupting tumor cell adhesion to the ECM as well as platelet aggregation itself.

Recently, a wide array of true viper and pit viper venoms consisting of peptides, termed disintegrins, have been shown to block integrin function and serve as potent inhibitors of platelet aggregation. Scarborough et al., “A GPIIb-IIIa-specific integrin antagonist from the venom of Sistrurus m. barbouri” J Biol Chem 266:9359-9362 (1991). The disintegrins comprise a portion of the molecules belong to the ADAM family of metalloproteinases. Such polypeptides are readily distinguished from the cobra-like venoms, which act as neurotoxins. Chiang et al., “Characterization of platelet aggregation induced by human colon adenocarcinoma cells and its inhibition by snake venom peptides, trigramin and rhodostomin” Br J Haematol 87:325-331 (1994). Most disintegrins contain the RGD cell adhesion recognition sequence, are rich in cysteine, and function to block platelet aggregation. Inhibition results from blocking the binding of the GPIIa/IIIb platelet integrin binding to ECM proteins such as fibrinogen and von Willebrand's factor. Wolfsberg et al., “ADAM, a novel family of membrane proteins containing A Disintegrin And Metalloprotease domain: multipotential functions in cell-cell and cell-matrix interactions” J Cell Biol 131:275-278 (1995); Chiang et al., “Characterization of platelet aggregation induced by human breast carcinoma and its inhibition by snake venom peptides, trigramin and rhodostomin” Breast Cancer Res Treat 33:225-235 (1995); Coller et al., “The anti-GPIIb-IIIa agents: fundamental and clinical aspects” Haemostasis 26 Suppl 4: 285-293 (1996). Since most disintegrins possess an RGD sequence, they are capable of further inhibiting the adhesive functions of other RGD-dependent integrins such as αᵥβ₅ (vitronectin receptors) and α₅β₁, a fibronectin receptor. Sheu et al., “Triflavin, an Arg-Gly-Asp-containing peptide, inhibits the adhesion of tumor cells to matrix proteins via binding to multiple integrin receptors expressed on human hepatoma cells” Proc Soc Exp Biol Med 213:71-79 (1996); and Nurden A, “New thoughts on strategies for modulating platelet function through the inhibition of surface receptors” Haemostasis 26 Suppl 4:78-88 (1996). However, by comparison with the integrins, other amino acid adhesion sequences such as Lys-Gly-Asp, may be operational in the disintegrins.

GIP-derived peptides may contain the sequence Gly-Glu-Gly (GEG) which may also be present in other proteins that play a role in tumor spreading and/or metastases. See FIG. 12 Sections I-III. GIP-derived peptides are therefore similar to the disintegrins and/or metalloproteinases and may represent models for designing novel and potent compounds with therapeutic value in the inhibition of platelet aggregation and the blockage of the tumor-induced platelet aggregation stage of metastasis due to their broad spectrum of reactivity with many integrins.

Using the GIP-derived matrix metalloproteinase-associated peptide sequences found by homology matching (See FIG. 12 Section II) the following consensus sequence may be constructed:

(SEQ ID NO:75)

LSX₁DX₂X₃X₄ACGEGX₅AX₆IX₇X₈GHX₉X₁₀X₁₁RHX₁₂X₁₃X₁₄
PX₁₅X₁₆PGVG,

wherein,

X₁=E or D; X₂=K, R, or L; X₃=L or H; X₄=L or M; X₅=A, S, or I;

X₆=D, E, or S; X₇=I or V; X₈=I or G; X₉=L or E; X₁₀=C or W; X₁₁=I or V;

X₁₂=E or N; X₁₃=M, I, or P; X₁₄=T, V, or I; X₁₅=V, E, or Y; X₁₆=N, G, or F.

However, in certain embodiments the present invention contemplates a matrix metalloproteinase-associated consensus sequence having all but one X amino acid substituted with a native GIP amino acid.

3. Extracellular Membrane Proteins

Integrins serve as receptors for ECM proteins and are known to participate in cell adhesion and migration (spreading) activities. Also, a Genebank match can be seen with a number of blood clotting factors which also interact with the integrins. Finally, matches were found with the platelet-associated proteins, such as the Von Willebrand Factor which is involved with platelet activation, aggregation, and metalloproteinases. Murawaki et al., “Serum tissue inhibitor of metalloproteinases in patients with chronic liver disease and with hepatocellular carcinoma” Clin Chim Acta 218:47-58 (1993). A further definition of integrin-associated ECM ligands include specific peptides including, but not limited to, collagen, laminin, fibronectin, fibrinogen, as well as the integrin α/β chain proteins such as α_{IIβ}β₃, α₁β₃ and αᵥβ₁. Mizejewski G, “Role of integrins in cancer: survey of expression patterns” Proc Soc Exp Biol Med 222:124-138 (1999). Thus, GIP-derived peptides share amino acid identity/similarity related to integrins, platelets, extracellular matrix proteins, and blood clotting factors which are involved in cell-to-cell and cell-to-ECM interactions. See FIG. 12 Section I.

Using GIP-derived extracellular matrix-associated peptide sequences found during homology matching the following consensus sequence may be constructed:

(SEQ ID NO:74)

LSEX₁KLLX₂CGX₃GX₄X₅X₆IX₇X₈X₉HLX₁₀IX₁₁HX₁₂X₁₃X₁₄
PX₁₅X₁₆PGVG,

wherein;

X₁=D, I, Q, R, or T; X₂=G, A, F, or P; X₃=E, R, S, or D; X₄=A, G, L, V, S, or Y;

X₅=A, G, L, P, S, T, or V; X₆=D, T, E, A, or N; X₇=I, F, or V; X₈=I, Y, L, V, or E;

X₉=G, L, R, or E; X₁₀=C, V, I, or P; X₁₁=R or K; X₁₂=E, Q, or R;

X₁₃=M, L, A, I, G, P, or F; X₁₄=T, S, P, A, R, or I; X₁₅=V, L, C, F, S, Y, or I; and

X₁₆=N, A, L, P, G.

However, in certain embodiments the present invention contemplates an extracellular matrix-associated consensus sequence having all but one X amino acid substituted with a native GIP amino acid.

Human AFP has been shown to display short amino acid sequences that show sequence identity to a variety of extracellular matrix (ECM) proteins. Mizejewski G, “Alpha-fetoprotein structure and function: relevance to isoforms, epitopes, and conformational variants” Exp Biol Med 226: 377-408 (2001); Mizejewski et al., “Alpha-fetoprotein growth inhibitory peptides: potential leads for cancer therapeutics” Mol Cancer Ther 2:1243-1255 (2003); and Mizejewski G, “Role of integrins in cancer: survey of expression patterns” Proc Soc Exp Biol Med 222:124-138 (1999). Therefore, GIP was subjected to cell adhesion studies employing ECM proteins.

Various ECM proteins were adsorbed to microliter plates and screened for their ability to serve as a substratum for enhanced tumor cell adhesion compared to non-coated microtiter plates. The ECM proteins differed in their ability to serve as attachment surfaces for two breast cancer cell types, human MCF-7 and murine 6WI-1. The mouse mammary 6WI-1 tumor cell attachment ranged from 20 to 60% for all ECM protein matrices with laminin demonstrating the greatest adhesion ability (60%). Fibrinogen ranked second as an adhesion factor with fibronectin and the collagens in close proximity. The non-protein attachment factors such as gelatin, chondroitin sulfate, and poly-lysine exhibited somewhat decreased cellular adhesion in comparison (20%-40%) to the ECM proteins. Using the MCF-7 cells, all ECM proteins were capable of supporting tumor cell adhesion with the exception of laminin. Cell adhesion to laminin in the MCF-7 cells ranged from 20 to 58%, with fibronectin achieving the highest percent attachment.

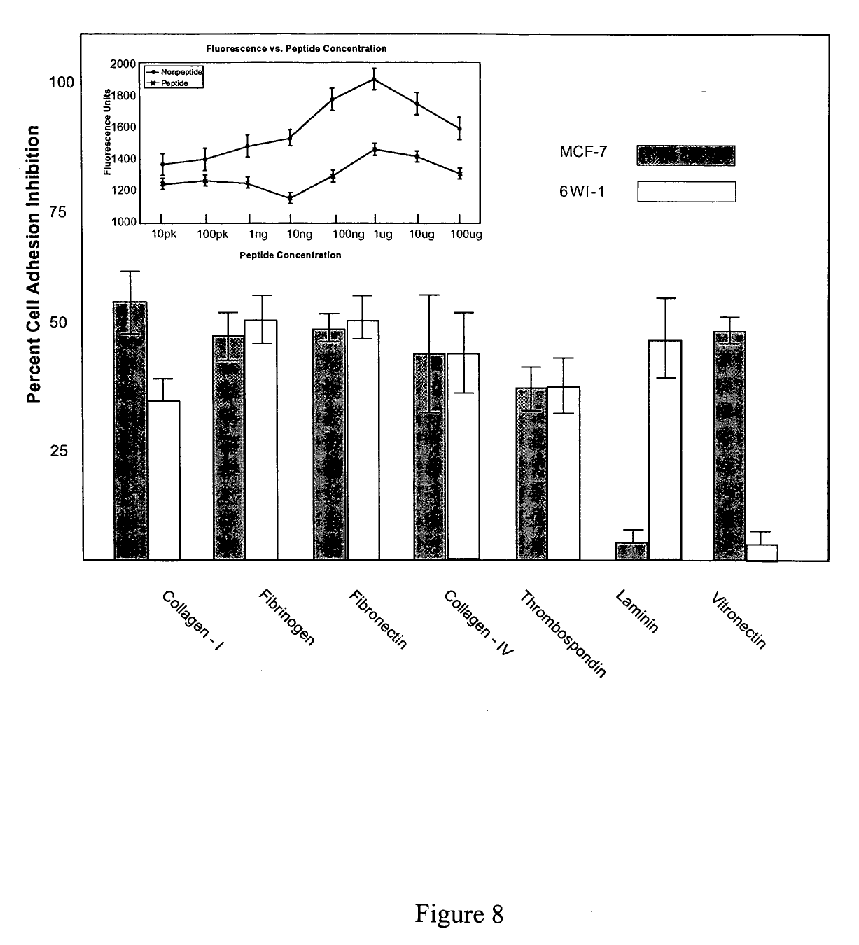

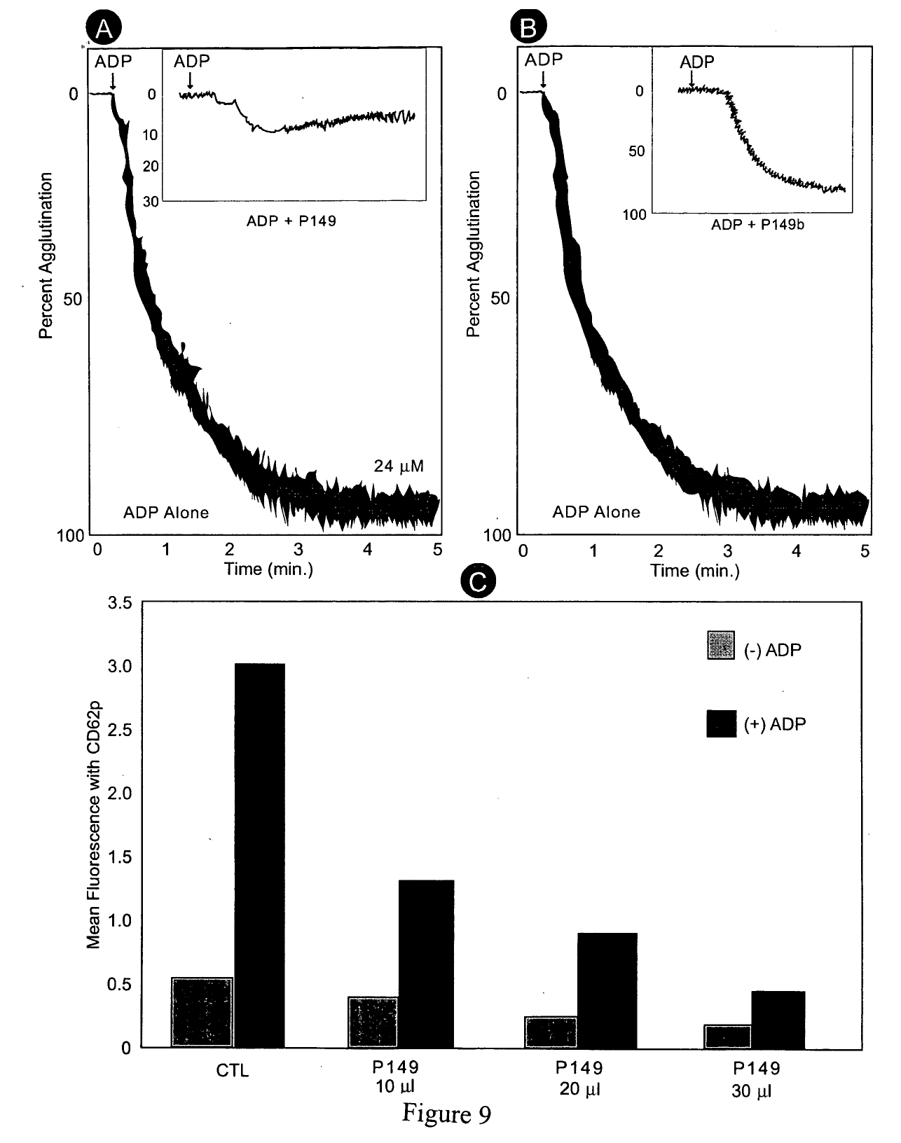

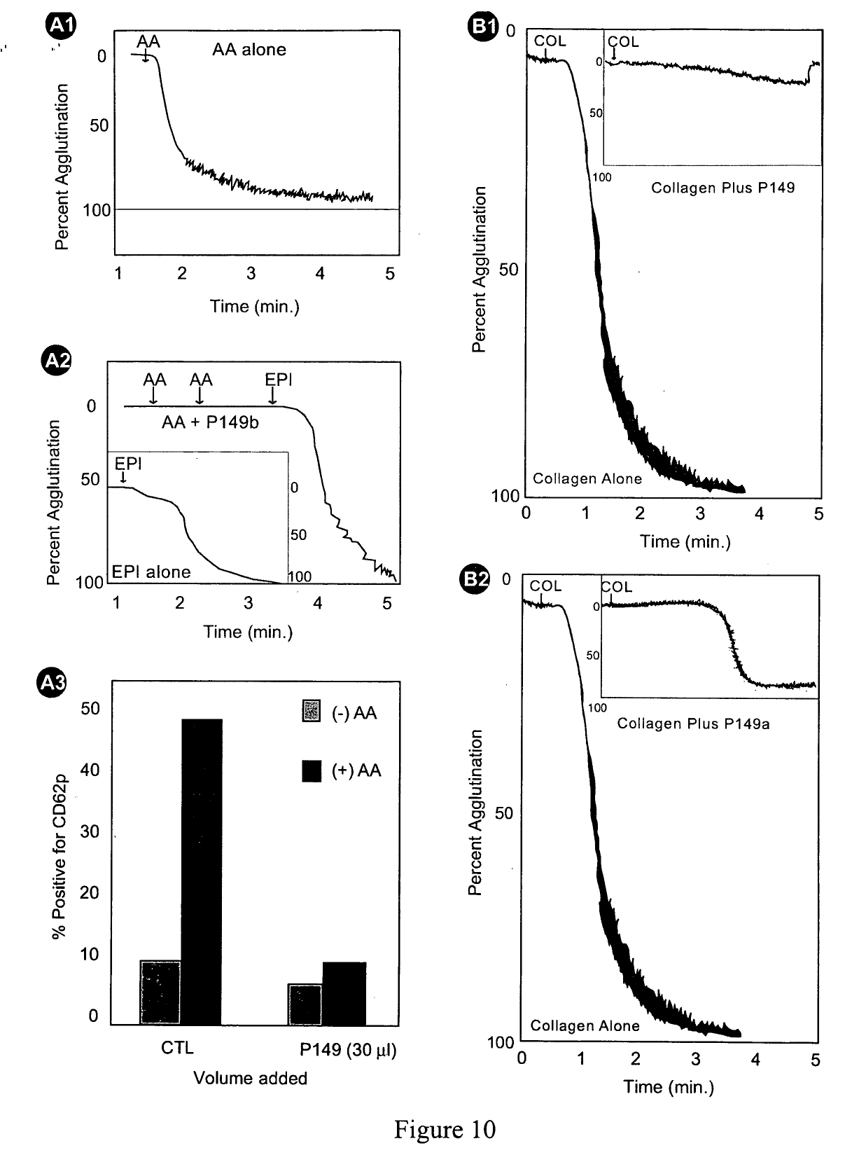

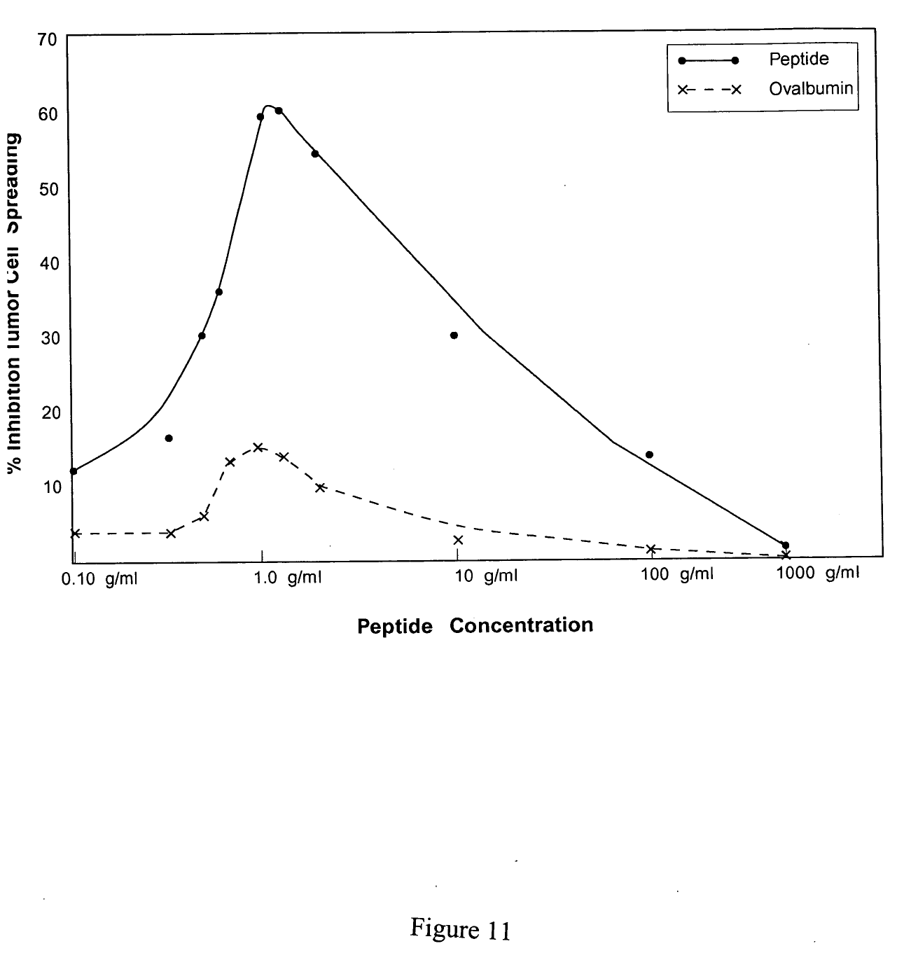

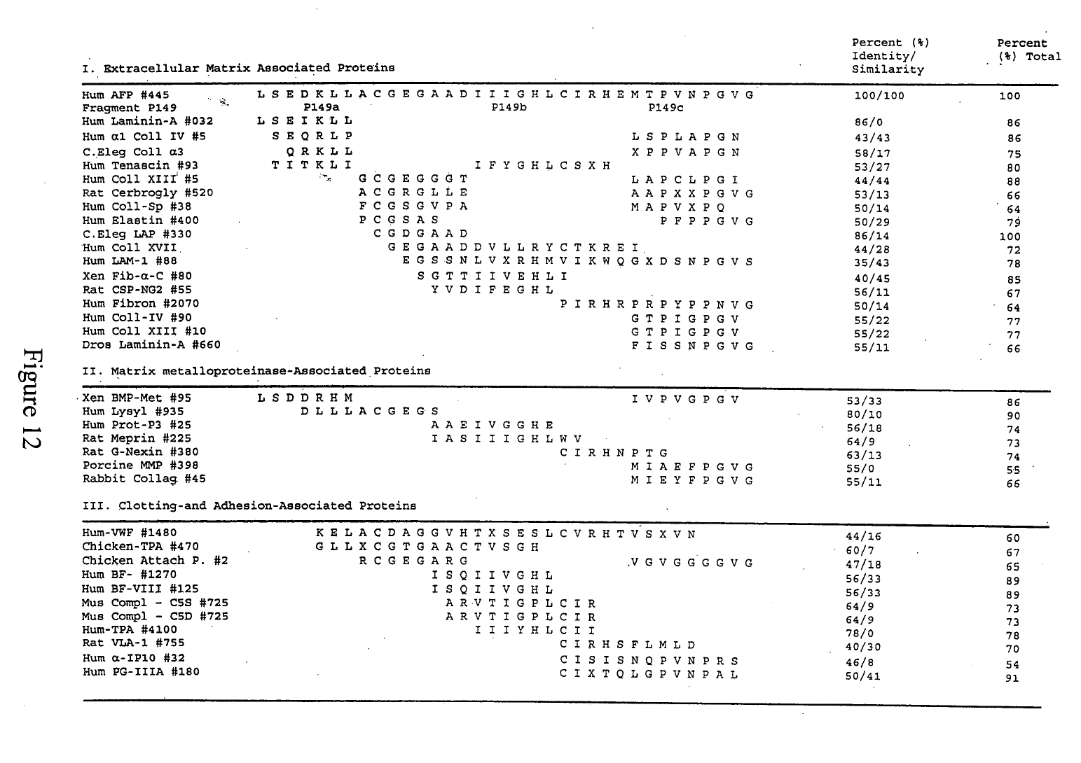

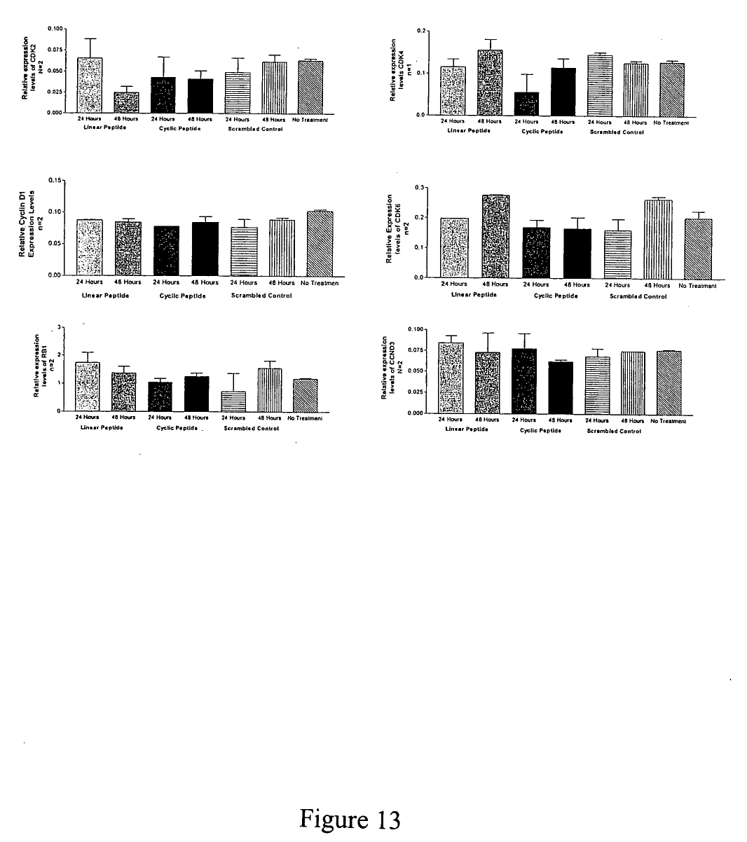

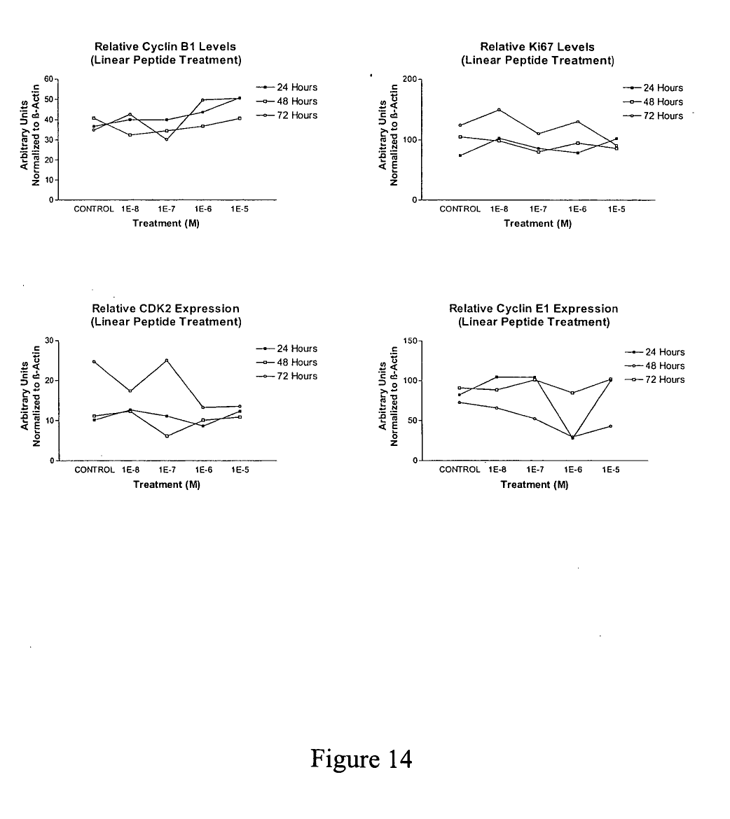

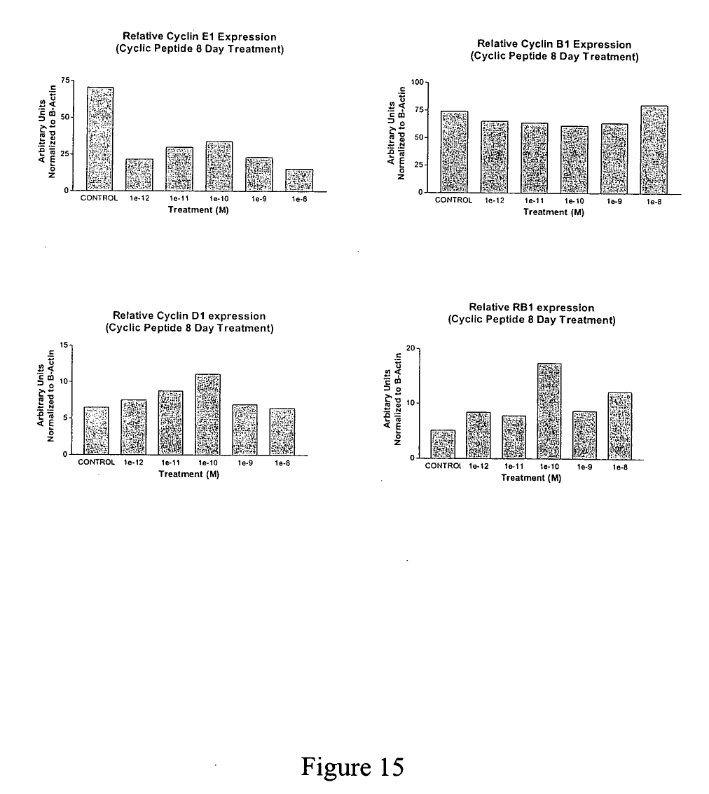

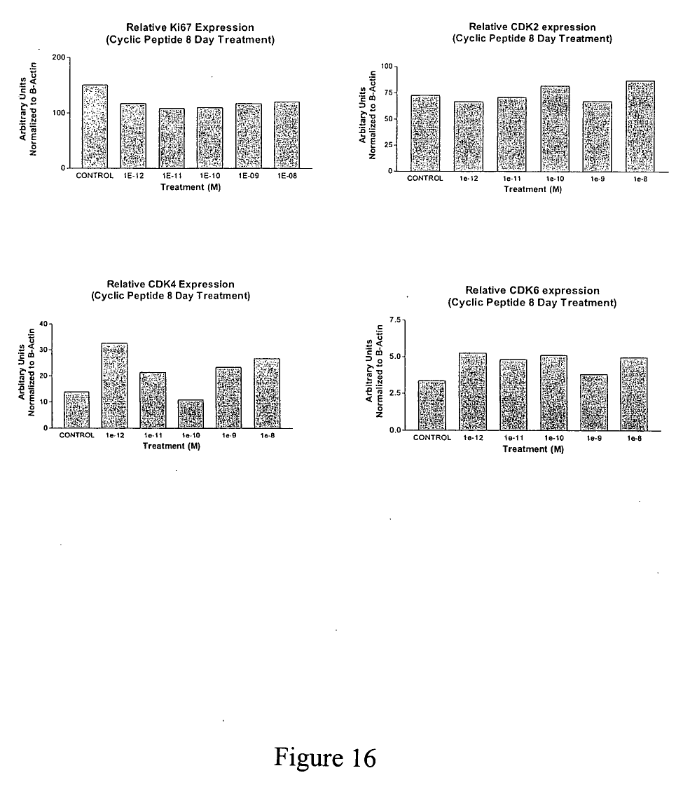

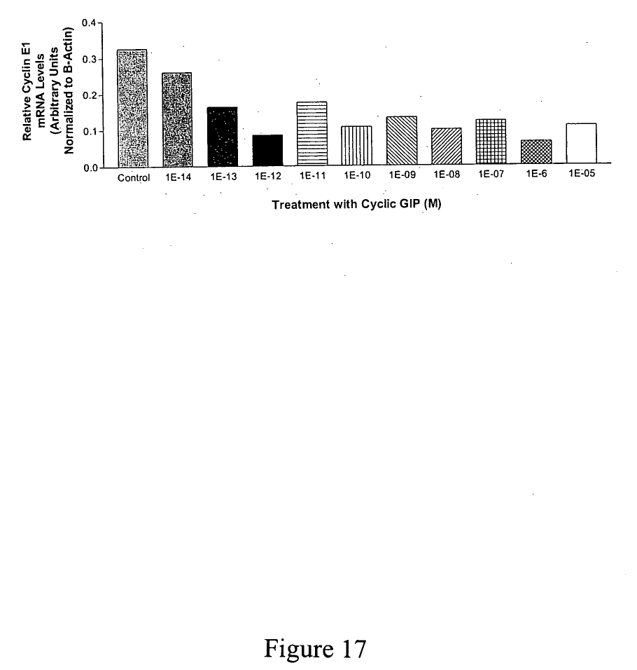

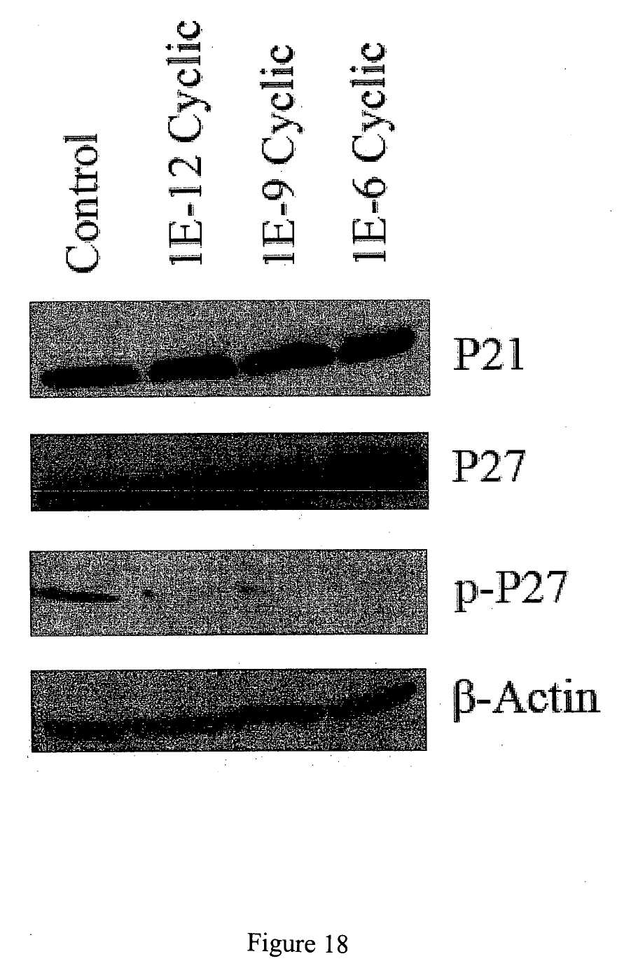

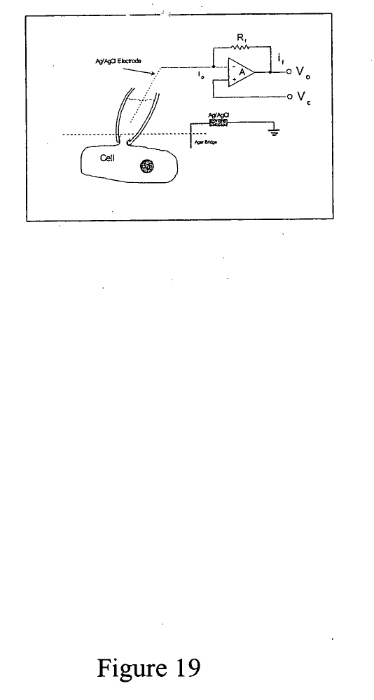

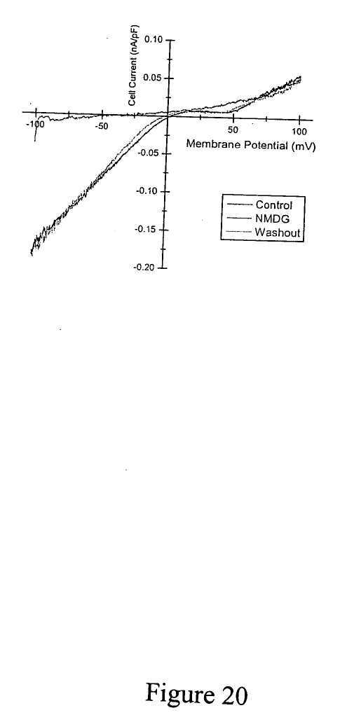

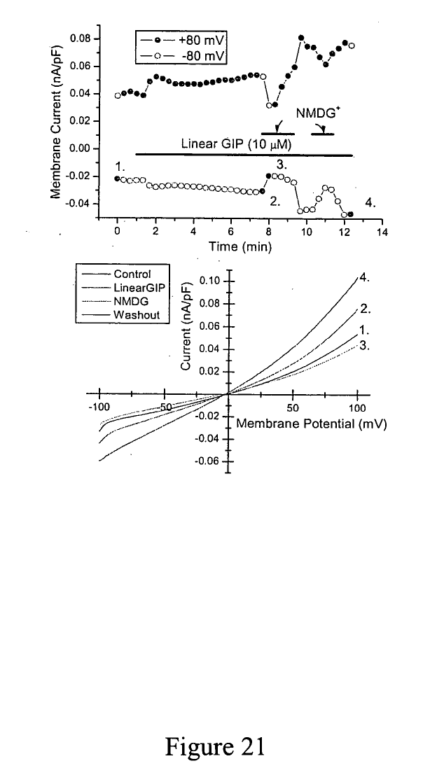

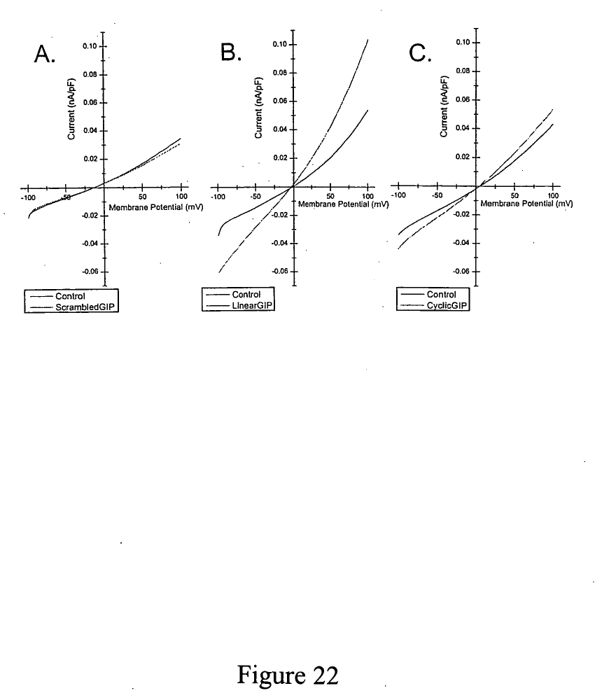

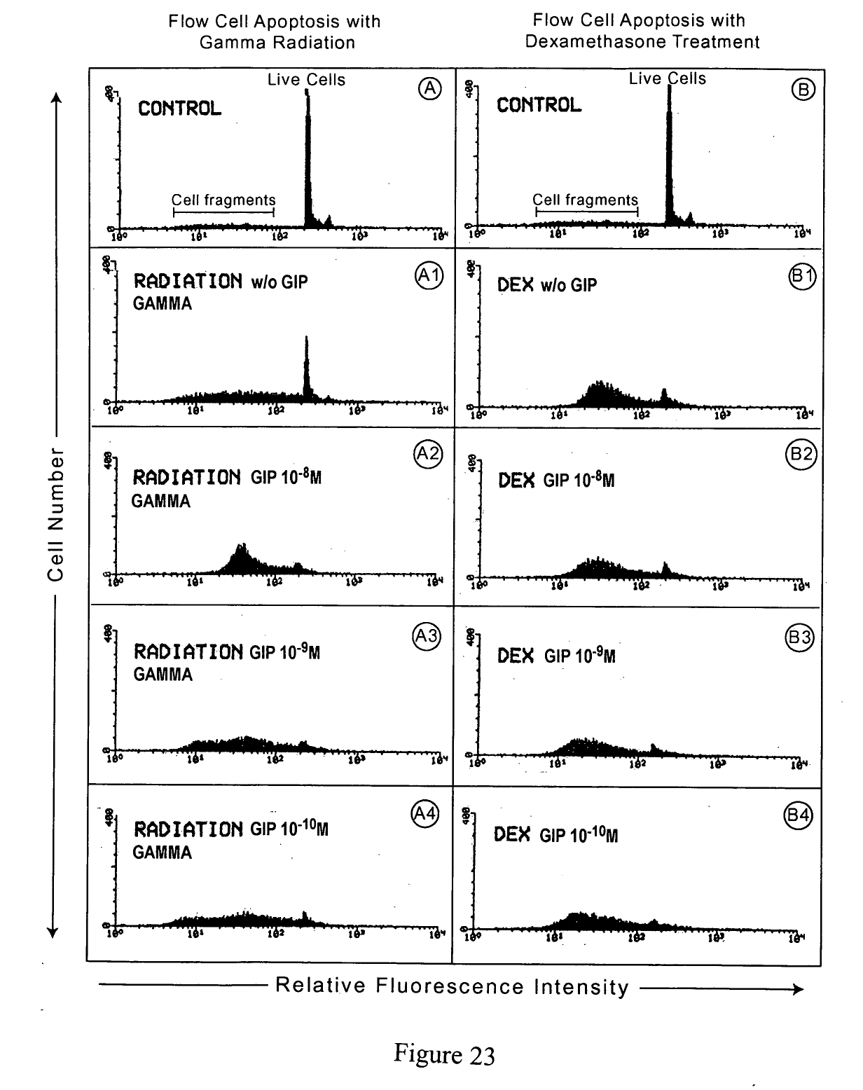

Laminin failed to support MCF-7 cell attachment (<5%), while collagens I and IV ranged from 30 to 50%, with fibrinogen displaying 20%. Interestingly, the AFP peptide (10 μg/ml) itself was capable of supporting 20 to 25% cellular adhesion with both tumor cell types. This attachment of GIP was a slightly weaker adhesion matrix than was gelatin, chondroitin sulfate, and poly-lysine which approximated 30-35%. Using P149, the optimal amount of peptide required to inhibit tumor cell adhesion was determined by absorbing soluble peptide (1.0 pg to 100 μg) to the walls of microtiter plates. A constant number of MCF-7 tumor cells (i.e., for example, 1×10⁵) were then added to the microtiter well and adhesion quantitated by fluorescence. See FIG. 8 (Inset). The greatest decrease in fluorescence units between peptide treated and nonpeptide plates was in the range of 1.0 μg to 10 μg P149/well. The quantitation of MCF-7 and 6WI-1 cell adhesion in the presence of peptide versus peptide-free media was then determined on ECM-coated microtiter plates using 3 μg GIP/well as a competitive inhibitor. The P149 peptide was added to the wells immediately after the cells were dispensed and then the mixture was incubated as described. The AFP peptide was capable of cell adhesion inhibition in most of the ECM proteins in both tumor cell lines. See FIG. 8. Inhibition of mouse and human tumor cell adhesion was somewhat equivalent using collagen IV, fibrinogen, fibronectin, and thrombospondin. However; inhibition was significantly different between laminin, collagen-I, and vitronectin when compared between the two tumor cell types. Human MCF-7 cells, in the presence of GIP, displayed substantial inhibition to vitronectin, while mouse tumor cells failed to respond. Likewise, mouse 6WI-1 cells demonstrated peptide inhibition to laminin adhesion but MCF-7 was not inhibited by this ECM protein. Overall, it was found that P149 peptide in the presence of tumor cells was found to competitively inhibit MCF-7 and 6WI-1 cell adhesion by 40-50%. Thus, the GIP was found capable of eliciting different attachment inhibitions dependent on the type of ECM protein coating the wall of the microtiter plate and also the tumor cell type involved. It was further observed that an albumin peptide control was not capable of inhibiting tumor cell adhesion and that anti-P149 peptide antibodies completely blocked the adhesion inhibition effect. Finally, it was observed that the addition of high Ca²⁺ levels in the culture media failed to influence either ECM adhesion to the microliter plates or the peptide inhibition of tumor cell adhesion to the ECM matrices.

Using the cell adhesion assay, AFP peptides were shown to influence tumor cell adhesion to a variety of ECM proteins. These ECM inhibitory adhesion events might be linked to integrin receptor interactions. Upon contact with the various ECM proteins, the integrins are known to induce a focal adhesion signal to influence matrix attachment. Mizejewski G, “Role of integrins in cancer: survey of expression patterns” Proc Soc Exp Biol Med 222:124-138 (1999). Each of the ECM proteins in this study was then matched to an integrin alpha/beta chain which is known to bind the ECM matrix. The α_{IIb} and αᵥ as well as β_{b} chains bind ligands such as fibronectin, fibrinogen, collagen I and IV, thrombospondin, vitronectin, and laminin. Ginsberg et al., “Ligand binding to integrins: common and ligand specific recognition mechanisms” Cell Differ Dev 32:203-213 (1990); Hynes R, “Integrins: versatility, modulation, and signaling in cell adhesion” Cell 69:11-25 (1992). This suggests that the AFP peptide with matched amino acid identities to various integrin α/β chains may serve as a competitive inhibitor to tumor and platelet-expressed integrin in order to affect the focal cell attachment signal. See Table 3.

TABLE 3
Genebank Matched GIPs To α/β Chains And Compared to GIP ECM Inhibition.

| | | AA Identity % | | ECM | ECM | Cell/Tissue |
|---|---|---|---|---|---|---|
| Integrin | GIP Amino Acid | | β- | Binding | to | and Tumor |
| Subunits | Sequence | α-chain | chain | Ligand | Tumor | Distribution |
| αᵥβ_{3A} | LSEDKLLACGEGAAD | 100 (9) | 47 (15) | Fib, VTN, | 40-50 | Melanomas and |
| | (SEQ ID NO: 86), | | | FbB, TSP | | angiogenic cell |
| | SEDKLLACG | | | | | |
| | (SEQ ID NO: 87) | | | | | |
| α_{M}β₂ | SEDKLLACG | 66.7 (9) | 43 (10) | FBN, | 50 | Immune, |
| | (SEQ ID NO: 87); | | | C3bi, | | Inflammatory |
| | LACGEGAADI | | | ICAM | | cells |
| | (SED ID NO; 88) | | | | | |
| αᵥβ₆ | SEDKLLA | 100 (7) | 50 (12) | FBN | 50 | Carcinoma cell |
| | (SED ID NO: 89) | | | | | virus associated |
| | | | | | | fusion |
| α₆β₁ | GEGAADII | 78 (9) | 75 (8) | LAM-1 | 10-45 | NSCL |
| | (SEQ ID NO: 90) | | | | | Carcinoma |
| αᵥβ₁ | SEDKLLA-CGEG | 100 (7) | 75 (4) | VTN, FBN | 40-50 | Analytic tumors |
| | (SEQ ID NO: 91) | | | | | |
| α₁β₁ | CGEGAADIIIGH | 43 (12) | 75 (8) | LAM | 10-45 | Breast |
| | (SEQ ID NO: 92) | | | COLL | | Carcinoma |
| α₁β₂ | CGEGAADIIIG (SEQ ID | 80 (11) | 43 (10) | FBN; C3i | 50 | Myeloid cells |
| (LFA-1) | NO: 93) | | | | | |
| α₄β₇ | GEGAADIIMTPVNPGV | 78 (9) | 56 (9) | FBN, | 50 | Endothelial X |
| | (SEQ ID NO: 94) | | | VCAM | | mucosal cells |
| | | | | MADCAM | | |
| α₃β₁ | DKLLACGEGAADIICGEG | 43 (14) | 75 (4) | FBN, | 30-55 | Most tumor cells |
| | (SEQ ID NO: 95) | | | COLL | | |
| | | | | LAM | | |
| αᵥβ₈ | IRHEMTOVNPG (SEQ ID | 67 (12) | 50 (12) | Not | Not | Reproductive |
| | NO: 96) | | | reported | done | Tissues |
| αᵥβ₅ | CGEGAADIIIGHLCIR- | 67 (12) | 80 (25) | VTN, FBN | 45-50 | Epithelium, |
| | HEMTOVNPGVGQ | | | | | carcinoma cells |
| | (SEQ ID NO: 97) | | | | | |
| α₆β₄ | IRHEMTPVPPGV | 78 (8) | 50 (12) | LAM-1 | 10-45 | Keratinocyte |
| | (SEQ ID NO: 98) | | | LAM-2 | | malignancy |
| α₂β₁ | IIGHLCIRHEMTPVNPGV | 53 (17) | 75 (8) | COLL | 10-55 | Epithelium, |
| | (SEQ ID NO: 99) | | | LAM | | endothelium |
| | | | | | | leucocytes |

COLL = collagen;
FBG = fibrinogen;
FBN = fibronectin;
Fib = fibrin;
LAM = laminin;
TSP = thrombospondin;
VTN = vitronectin;
VWF = von Willebrand's factor;
C3i = Complement Factor-3 inhibited.

Since the summary data shows integrin expression on various tumor cells, the observed cell adhesion inhibition suggests that GIP may be serving as a decoy floating (disintegrin) integrin for tumor cells. Inhibition of tumor cell adhesion to the ECM proteins would subsequently impair the ability of tumor cells to spread and metastasize.

4. Platelet Aggregation

Blood platelets are usually recognized as a primary facilitator of blood clotting. For example, following the addition of an agonist (i.e., for example, adenosine diphosphate), activation of platelet aggregation proceeds in a multi-step process which can be monitored by a continual decrease in light transmission reflection. First, the agonist causes the platelet to change shape (i.e., for example, by inducing changes in the cytoskeletal structure). Second, the platelets then form into increasingly larger clumps. Third, aggregation reaches a maximum plateau. Further, the aggregation profile can be divided into two phases; (a) primary aggregation, which is a reversible process; and (b) secondary aggregation, which is irreversible, and associated with granule release and thromboxane production. Depending on the agonist the secondary aggregation phase, may or may not, liberate arachidonate for thromboxane A₂ formation. Whittle et al., “Specificity between the anti-aggregatory actions of prostacyclin, prostaglandin E₁ and D₂ on platelets” In: Mechanisms of stimulus—response coupling in platelets, Eds. V. V. K., MacIntyre D E, Scully M F., New York and London: Plenum Press. pp. 109-115 (1985); and Hamberg et al., “Thromboxanes: a new group of biologically active compounds derived from prostaglandin endoperoxides” Proc Natl Acad Sci USA 72:2994-2998 (1975). In one embodiment, the present invention contemplates that GIP-derived peptide sequences are capable of affecting all three platelet aggregation phases.

The secondary platelet aggregation step stimulates arachidonic acid (AA) release and promotes its subsequent metabolism. GIP was identified as a potent inhibitor of AA-induced platelet aggregation causing the transformation of cyclo-oxygenases to endoperoxidases. Collagen also induces products of the cyclo-oxygenase pathway (i.e., for example, stimulation of AA release) but does not produce the primary platelet aggregation step.

The release of platelet granules are also associated with the secondary phase of aggregation. Platelets release at least four types of granules; 1) α-granules which contain fibrinogen, fibronectin, and thrombospondin; 2) coagulation factor granules; 3) dense granules containing serotonin, ATP, ADP, and Ca²⁺ ions; and 4) peroxisomes containing catalase and phosphatases. Epinephrine, unlike ADP, does not cause a shape change nor the primary aggregation step. Consequently, GIP was not capable of inhibiting epinephrine-induced platelet aggregation. Although it is not necessary to understand the mechanism of an invention, it is believed that GIP may not interact with the platelet cell surface binding sites employed by epinephrine to induce aggregation. One hypothesis suggests that these binding sites might be distinct from those used by ADP, AA, or collagen.

The GIP platelet aggregation inhibition data are listed in Table 4 in accordance with their platelet expressed integrin subunit chains and their specific ECM binding ligands.

TABLE 4
Platelet Associated Integrin Comparison To GIP Amino Acid Sequences.

| Integrin | | | | P149 | | |
|---|---|---|---|---|---|---|
| Subunits | | | | Sub- | Platelet | |
| And | Cell/Tissue | ECM/cell | | segment | Aggregation | Tumor/tissue |
| Original | Distribution | Binding | GIP Amino Acid | (includes | Inhibition | Expression |
| Names | Of Integrins | Ligand | Sequence Stretch | overlaps) | (%) | Of Integrins |
| α₂β₁ | epithelium | collagen | GEGAADIIIG | P149a, B | >90 | Animal |
| VLA-2 | endothelium | platelet | (SEQ ID NO: 100) | | | mammary |
| (GP1a) | leucocytes | | | | | carcinomas; |
| | platelets | | HLCIRHEMTPVNPG | P149b, C | | Human breast |
| | | | (SEQ ID NO: 101) | | | cancers |
| α₅β₁ | endothelium | FBN | DIIIGHLC | P149b | >70 | During growth |
| VLA-5 | hepatocytes | platelet | (SEQ ID NO: 102) | | | and |
| (GP1c) | platelets | | | | | tumorigenesis |
| α₆β₁ | most cells | LAM | GEGAADIII | P149a, B | >80 | Breast, liver, |
| VLA-6 | Epithelium | platelet | (SEQ ID NO: 103) | | | lung carcinoma |
| | endothelium | | DIIGHLC | P149b | | |
| | platelets | | (SEQ ID NO: 104) | | | |
| α_{IIb}β₃ₐ | granulocytes | FBN | LSEDKSSACGEGAA | P149a, B | >96 | Platelet |
| Major | monocytes | FIB | (SEQ ID NO: 105) | | | aggregation |
| Platelet | megakaryocytes | VTN | GHLCIRHE | P149b | | requirement |
| Receptor | platelets | VWF | (SEQ ID NO: 106) | | | |
| | | TSP | | | | |
| αᵥβ₃ | osteoclasts | FBN, FIB, | EDKLLAC | P149a | >70 | Melanoma |
| VTN | endothelium | FBG | (SEQ ID NO: 107) | | | Angiogenesis |
| receptor | fibroblasts | COLL | GHLCIRH | P149b | | |
| | | VWF | (SEQ ID NO: 108) | | | |
| | | TSP | | | | |

COLL = collagen;
FBG = fibrinogen;
FBN = fibronectin;
FIB = fibrin;
LAM = laminin;
TSP = thrombospondin;
VTN = vitronectin;
VWF = von Willebrand's factor;
C3i = Complement Factor-3 inhibited.

The data suggest that GIP-derived peptides showed the greatest inhibitory potencies toward the various activating agents including, but not limited to, collagen, ADP, and arachidonic acid. See Table 5.

TABLE 5
GIP-Derived Peptide Inhibition Of Induced Platelet Aggregation

| | Inhibition (%) Of Platelet Aggregation | | | |
|---|---|---|---|---|
| | Adenosine | Arachidonic | Collagen | |
| Peptide Inhibitors | Diphosphate | Acid (AA) | (COLL) | |
| 30 & 100 μM | (ADP) 2.5 μM | 0.3 mM | 2 μg/ml | Epinephrine |
| P149 (34-mer) | 80-90 | 100 | 90 | No effect |
| P149a (12-mer) | 30-40 (biphasic | No effect | No effect | No effect |
| | only) | | | |
| P149b (15-mer) | 95-98 | 100 | 90 | No effect |
| P149c (8-mer) | No effect | No effect | No effect | No effect |
| α2-antiplasma | 85-90 | 100 | 90 | No effect |
| (positive control) | | | | |
| P236 (human | 80-90 | Shape change only | 60-80 | No effect |
| albumin protein | | | | |
| homology control) | | | | |
| P192 | No effect | No effect | No effect | No effect |
| (negative control) | | | | |
| Risotcetin | 100 | 100 | 100 | 100 |
| (positive control) | | | | |

P149a and P149b also exhibited significant aggregation inhibition (>70%). In contrast, the carboxy-terminal P149c appeared to lack inhibitory capabilities regarding platelet aggregation. It was not surprising that the P149c lacks this inhibitory capability since few Genebank peptide identities were found.

Using the found GIP-derived clotting and adhesion-associated proteins by homology matching shown in FIG. 12C allow the following consensus sequence to be constructed:

(SEQ ID NO:76)

X₁X₂LX₃CX₄X₅GX₆X₇X₈X₉X₁₀X₁₁GHLCIRX₁₂X₁₃X₁₄X₁₅
PX₁₆NPX₁₇X₁₈G,

wherein,

X₁=K or G; X₂=E or L; X₃=A or R; X₄=D or G; X₅=A, T, or E; X₆=V, I, or T;

X₇=V, A, R, or S; X₈=H, C, G, Q, R or D; X₉=T, I, or V; X₁₀=V, I, or T;

X₁₁=S, V, or I; X₁₂=H, I, or T; X₁₃=T, S, Q, or E; X₁₄=N, F, N, L, or M;

X₁₅=S, G, L, Q, or T; X₁₆=V, G, or L; X₁₇=G, R, or A; and

X₁₈=V, S, or L.

However, in certain embodiments the present invention contemplates a clotting and/or adhesion-associated consensus sequence having all but one X amino acid substituted with a native GIP amino acid.

B. GIP Subfragments Having Cation Channel Effects

Voltage-gated ion channels are known to play a role in the electrical activity of mammalian cells. Large families of voltage-gated ion channels (i.e., for example, potassium, calcium, and sodium channels) have been identified. These ion channels have been the target of significant pharmacologic study, due to their potential role in a variety of pathological conditions.

Calcium channels are present in various tissues, have a central role in regulating intracellular calcium ion concentrations, and are implicated in several processes (i.e., for example, neurotransmitter release, muscle contraction, pacemaker activity, secretion of hormones and other substances, etc.). Thus, changes in calcium influx into cells which are mediated through calcium channels have been implicated in various human diseases such as disorders of the central nervous system and cardiovascular disease.

For example, changes to calcium influx into neuronal cells may be implicated in neurological conditions such as, but not limited to, epilepsy, stroke, brain trauma, Alzheimer's disease, multi-infarct dementia, other classes of dementia, Korsakoff's disease, neuropathy caused by a viral infection of the brain or spinal cord (e.g., human immunodeficiency viruses, etc.), amyotrophic lateral sclerosis, convulsions, seizures, Huntington's disease, amnesia, or damage to the nervous system resulting from reduced oxygen supply, poison or other toxic substances. Goldin et al., “Calcium channel antagonists and methodology for their identification” U.S. Pat. No. 5,312,928 (herein incorporated by reference).

Additionally, changes to calcium influx into cardiovascular cells may be implicated in conditions such as, but not limited to, cardiac arrhythmia, angina pectoris, hypoxic damage to the cardiovascular system, ischemic damage to the cardiovascular system, myocardial infarction, and congestive heart failure. Goldin et al., (supra). Other pathological conditions associated with elevated intracellular free calcium levels include muscular dystrophy and hypertension. Steinhardt et al., “Methods for screening compounds to determine calcium leak channel inhibition activity” U.S. Pat. No. 5,559,004 (herein incorporated by reference). While there has been limited success in expressing DNA encoding rabbit and rat calcium channel subunits, little is known about human calcium channel structure, function and gene expression. Additionally, there is limited knowledge in the art of the role of calcium channel types in cell growth control and abnormalities of calcium channels leading to cancer development.

In addition to the implication of calcium channels in animal (including human) diseases, a number of compounds which are currently used for treating various cardiovascular diseases in animals (including humans) are believed to exert their beneficial effects by modulating the functions of voltage-dependent calcium channels present in cells, such as, but not limited to, cardiac cells and vascular smooth muscle cells. Nonetheless, the pharmacology of compounds which interact with calcium channels is not understood. This lack of understanding, together with the limited knowledge in the art of: i) the human calcium channel types; ii) the molecular nature of the human calcium channel subtypes; and iii) the limited availability of pure preparations of specific calcium channel subtypes to use for evaluating the efficacy of calcium channel-modulating compounds has hampered the rational testing and screening of compounds that interact with the specific subtypes of human calcium channels to have desired therapeutic effects.

As discussed above, GIP inhibits growth of various human tumors, including those of breast and prostate. Caceres et al., Anticancer Res. 22:2817 (2002). The cellular mechanisms by which GIP achieves such growth inhibition are largely unknown. Nonetheless, the rapid translocation of GIP into the cytoplasm and eventually to the cell's perinuclear region, its properties as a microtubule-associated protein and its capacity to rapidly effect changes in cell morphology suggest that GIP may affect ionic and osmotic balance of cells. Mizejewski et al., Mol. Cancer. Ther. 2:1243 (2003); Mizejewski et al., Cell Biol. Intl 28:913 (2004). Indeed, the importance of ions in controlling cell proliferation has long been an area of interest among tumor biologists. cf., Boyton, McKeehan and Whitfield, eds. Ions. Cell Proliferation and Cancer. Academic Press, New York (1982).

However, specific ion channels and cellular mechanisms involved in intracellular ionic homestatsis comprise broad possibilities. These include, but are not limited to; i) membrane Cl⁻ channels: Wondergem et al., In: Rouzaire-Dubois, Benoit & Dubois, eds. Ion Channels and Physiopathologies of Nerve Conduction and Cell Proliferation pp. 173-186) 2002); ii) membrane K⁺ channels; Wonderlin et al., J Membr. Biol. 154:91 (1996); and iii) membrane Ca²⁺ channels Mignon et al., In: Rouzaire-Dubois, Benoit & Dubois, eds. Ion Channels and Physiopathologies of Nerve Conduction and Cell Proliferation. pp. 103-119 (2002). However, it is only recently that the molecular identity of K⁺ channels and Cl⁻ channels, which are involved in the regulation of membrane potential and cell volume, have been established as these relate to control of cell proliferation. Pardo et al., Physiology 12:285 (2004); and Lemonnier et al., Cancer Res. 64:4841 (2004), respectively. In one embodiment, the present invention contemplates that GIP and/or GIP-derived peptides modulate intracellular calcium-potassium balance by interacting with a cation channel. In one embodiment, the cation channel comprises a calcium channel. In another embodiment, the cation channel comprises a potassium channel. In one embodiment, the cation channel comprises a calcium-activated potassium channel.

The transient receptor potential family of ion channels (TRPs) comprise non-selective cation channels (i.e., for example, a non-selective calcium channel) whose protein subunits display a limited homology to the first-discovered member of the superfamily, the Drosophila TRP. These include, but are not limited to the canonical subfamily (TRPC), the vanilloid subfamily (TRPV) or the melastatin subfamily (TRPM). The transmembrane topology of TRPs is similar to the voltage-gated ion channels and comprises six transmembrane-spanning regions (TM1-6), cytoplasmic N- and C-termini, and a pore region between TM5 and TM6. The N-terminus of TRPV and TRPC contain multiple ankyrin binding repeats. These do occur in TRPM, which have relatively long N- and C-termini, some of which comprise entire enzyme domains. Nilius et al., Endothelium 10:5 (2003).

Recent studies show that human lymph node carcinoma of the prostate cells (LNCaP) contain store-operated Ca²⁺ channels that have been attributed to TRP ion channels, likely TRPC1 and/or TRPV6. Vanden Abeele et al., Cell Calcium 33 (2003). Others have shown that LNCaP cells also express the vanilloid receptor ion channel (i.e., for example, TRPV1) and that it is functionally active. Sanchez et al., Eur. J. Pharmacol. (In press, 2005).

Potassium channels are known to play a role in maintaining a proper intracellular ionic balance, as well as cell membrane electrical potential. For example, the relative infux and efflux of potassium, sodium, and calcium is believed responsible for the generation of action potentials in neuronal cells. Further, a proper homeostatic balance between potassium, sodium, and calcium is also believed responsible to maintain intracellular ionic homeostasis. Consequently, the effects of K⁺ channel modulators including, but not limited to, tetraethylammonium, 4-aminopyridine and diazoxide, and high extracellular K⁺ may affect cell growth and agonist-induced intracellular Ca²⁺ mobilization. Lee et al., “Inhibition of cell growth by K⁺ channel modulators is due to interference with agonist-induced Ca²⁺ release” Cell Signal. November; 5(6):803-809 (1993). In this study, two human brain tumour cell lines, U-373 MG astrocytoma and SK-N-MC neuroblastoma, were used as model cellular systems. K⁺ channel modulators and increased extracellular K⁺ concentration inhibited tumour cell growth in a dose-related fashion in both cell lines. In addition, agonist (carbachol or serum)-induced intracellular Ca²⁺ mobilization was also blocked by the pretreatment of growth-inhibitory concentrations of K⁺ channel modulators and high extracellular K⁺. Thus, these results suggest that K⁺ channel modulators are effective inhibitors of brain tumour cell growth and that their growth regulation may be due to the interference with the intracellular Ca²⁺ signalling mechanisms. Other studies have investigated methods of delivering a potassium channel activator to an abnormal brain region and/or a malignant tumor. Black et al., “Method for using potassium channel activation for delivering a medicant to an abnormal brain region and/or malignant tumor” United States Patent Application Publ No. 20050153940 (herein incorporated by reference).

The calcium-activated potassium channel (K_{Ca}) is believed to be an important regulator of blood vessel tone by smooth muscle regulation. Nelson et al., “Physiological roles and properties of potassium channels in arterial smooth muscle” Am. J. Physiol. 268(4 Pt 1): C799-822 (1995); and Bang et al., “Nitroglycerin-mediated vasorelaxation is modulated by endothelial calcium-activated potassium channels” Cardiovasc. Res. 43(3): 772-78 (1999). The K_{Ca} channel is ubiquitously distributed in tissues. K_{Ca} activity is believed triggered by membrane depolarization and enhanced by an increase in cytosolic (i.e., intracellular) calcium di-cation (Ca²⁺). An extracellular increase in Ca²⁺ is detected by a K_{Ca} subunit directed towards the cytoplasm in the cell. This interaction thereby promotes potassium cation flux through these channels. Brian et al., “Recent insights into the regulation of cerebral circulation, Clin. Exp. Pharmacol. Physiol. 23(6-7): 449-57 (1996). Minoxidil sulfate and chromakalim are reported to be activators of K_{ATP}. Wickenden et al., “Comparison of the effects of the K(+)-channel openers cromakalim and minoxidil sulphate on vascular smooth muscle” Br. J. Pharmacol 103(1): 1148-52 (1991).

It is also known that large-conductance voltage and K_{Ca} channels play a role in modulating neural activity. Further, in most mammalian tissues, activation of beta-adrenergic receptors and protein kinase A (PKAc) may increase K_{Ca} channel activity, contributing to sympathetic nervous system/hormonal regulation of membrane excitability. Recently, an association of the beta₂-adrenergic receptor (beta₂AR) with the pore forming alpha subunit of K_{Ca} and an A-kinase-anchoring protein (AKAP79/150) for beta₂ agonist regulation has been reported. These studies indicate that beta₂AR can simultaneously interact with both K_{Ca} and L-type Ca²⁺ channels (Caᵥ1.2) in vivo, which enables the assembly of a unique, highly localized signal transduction complex to mediate Ca²⁺- and phosphorylation-dependent modulation of K_{Ca} current. One hypothesis suggests that G protein-coupled receptors act as a scaffold to couple two families of ion channels into a physical and functional signaling complex to modulate beta-adrenergic regulation of membrane excitability. Liu et al., “Assembly of a Ca²⁺-dependent BK channel signaling complex by binding to beta₂ adrenergic receptor” EMBO J. 23:2196-205 (2004).

For example, the charybdotoxin receptor, purified from bovine tracheal smooth muscle, consists of two subunits (alpha and beta) and, when reconstituted into planar lipid bilayers, forms functional high conductance Ca²⁺-activated K⁺ channels. The channel is believe to be a protein of 191 amino acids that contains two hydrophobic (putative transmembrane) domains and bears little sequence homology to subunits of other known ion channels. Studies have shown that these high conductance Ca²⁺-activated K⁺ channel exists as a multimer containing both alpha- and beta-subunits. Furthermore, charybdotoxin is specifically and covalently incorporated into when cross-linked to the channel. Knaus et al., “Primary sequence and immunological characterization of beta-subunit of high conductance Ca(2+)-activated K+ channel from smooth muscle” J Biol. Chem. 269:17274-8 (1994).

The MaxiK channel (K_{V,Ca}) beta subunit is reported to dramatically increase the apparent calcium sensitivity of the alpha subunit of MaxiK channels when probed in the micromolar intracellular calcium ([Ca²⁺]ᵢ) range. Analysis in a wide range of [Ca²⁺]ᵢ revealed that this functional coupling is exquisitely modulated by [Ca²⁺]ᵢ. Ca²⁺ ions switch MaxiK alpha+beta complex into a functionally coupled state at concentrations beyond resting [Ca²⁺]ᵢ. At [Ca²⁺]≦100 nM, MaxiK activity becomes independent of Ca²⁺, is purely voltage-activated, and its functional coupling with its beta subunit is released. The functional switch develops at [Ca²⁺]ᵢ that occur during cellular excitation, providing the molecular basis of how MaxiK channels regulate smooth muscle excitability and neurotransmitter release. Meera et al., “A calcium switch for the functional coupling between alpha (hslo) and beta subunits (KV,Ca beta) of maxi K channels” FEBS Lett. 382:84-88 (1996).

The K_{Ca} beta-subunit has been cloned and expressed from human brain tissue. These studies used an open reading frame encoding a 191-amino acid protein possessing significant homology to a previously described subunit cloned from bovine muscle. The gene for the bovine muscle subunit is located on chromosome 5 at band q34 (hslo-beta). Brain subregions in which beta-subunit mRNA expression was relatively high are the hippocampus and corpus callosum. Further, the coexpression hslo-beta mRNA together with hslo-alpha subunits in either Xenopus oocytes or stably transfected HEK 293 cells also give rise to Ca(2+)-activated potassium currents with a much increased calcium and/or voltage sensitivity. Consequently, it is believed that these beta-subunits can play a functional role in the regulation of neuronal excitability by tuning the Ca2+ and/or the voltage dependence of alpha-subunits. Tseng-Crank et al., “Cloning, expression, and distribution of a Ca(2+)-activated K+ channel beta-subunit from human brain” Proc Natl Acad Sci USA 93:9200-9205 (1996).

Although it is not necessary to understand the mechanism of an invention, it is believed that GIP affects membrane ion currents in LNCaP cells and may explain GIP's action on membrane TRP channels. Observations of a GIP-induced non-selective cationic current in LNCaP cells and increased slope conductance of the plasma membrane further support this hypothesis.

In some embodiments, GIP-derived peptides were identified by homology matching having a cation channel (i.e., for example, K_{Ca}, calcium, potassium, or sodium) gating modulation functionality comprising:

| | |
|---|---|
| LSEDKLLACGEGAADIIIGHLCIRHEMTPVNPGVG | (SEQ ID NO:2) |
| LSEDKLLACGEGD | (SEQ ID NO:16; linear) |
| EGAADIIIGHLCIRHE | (SEQ ID NO:17; linear) |
| EMTPVNPGVGN | (SEQ ID NO:18; linear) |
| DCIRHEMTPVNPGVGD | (SEQ ID NO:19; linear) |
| CQLSEDKLLAC | (SEQ ID NO:20; cyclic) |
| CGEGAADIIIGHLC | (SEQ ID NO:21; cyclic) |
| CIRHEMTPVNPGVGQC | (SEQ ID NO:22; cyclic) |

A comparative homology analysis allows the construction of the following consensus sequence:

LSEDKLLACGEGX₁QDIIIGHX₂CIRHEMTPVNPGVG (SEQ ID NO:77), wherein X₁=A or D; and X₂=L or D. However, in certain embodiments the present invention contemplates a cation channel consensus sequence having all but one X amino acid substituted with a native GIP amino acid.

Normal epithelial cells, the source of most human solid tumors, use an apical junction complex to maintain their polarity, initiate and maintain cell-cell contacts, and regulate fluxes of molecules across the cellular space. Decreases in extracellular calcium concentration have been shown to induce disassembly of these epithelial tight junctions by reorganization of actin filaments. Ivanov et al., Mol Biol Cell 15:2639-2651 (2004). During carcinogenesis these calcium dependent junctional complexes disappear.

It has recently been discovered that P149 may affect the membrane channels that supply cells with calcium from the extracellular environment. For example, it has been suggested that human lymph node carcinoma of the prostate cells (LNCaP) contain non-selective cationic Ca²⁺ channels that have been attributed to the transient receptor potential (TR) family of ion channels. Using LNCaP cells, whose proliferation is negatively affected by P149, it has been shown that P149 increases slope conductance of the plasma membrane. Vakharia et al, “Human alpha-fetoprotein peptides bind estrogen receptor and estradiol, and suppress breast cancer” Breast Cancer Res. & Treat 63:41-52 (2000).

Although it is not necessary to understand the mechanism of an invention, it is believed that GIP-derived peptides may alter the activity of the endogenous TRP calcium channels or are forming pores themselves that allow calcium to enter the cell. It is further believed that de novo pore forming ability is possible because linear forms of GIP-derived peptides are most effective when they form multimers and/or oligomers. GIP-derived peptide oligomerization is thought to facilitate cation channel pore formation, and there is significant amino acid homology between GIP-derived peptides and TRP calcium channel amino acid sequences.

The data presented herein show that addition of either linear or cyclic GIP increases conductance of the plasma membrane of LNCaP cells. The absence of any effect by the scrambled GIP sequence indicates specificity of the intact GIP molecule in this regard. The results also suggest that GIP increases activity of non-selective cation channels of the plasma membrane, because the increases in slope conductance (i.e., for example, a decrease in membrane resistance) occurred without changes in reversal potentials, which were at or near zero. Thus, GIP may activate existing non-selective cation channels in the plasma membrane, which include the various TRP channels, or GIP may itself function as an ion channel or porin.

Enterotoxin and antibiotic peptides have been reported to function as ionic channels Hardy et al., J. Med. Microbiol. 48:235 (1999); and Ye et al., J. Biochem. 136:255 (2004). In one embodiment, GIP modulates the activity of cation channels, including, for example, TRP channels. The mechanisms by which TRP channels are activated or modulated are largely unknown. Nonetheless, it is well established that TRP channels respond to; i) ligand gating (i.e., for example, TPV1 activation by capsaicin): Caterina et al, Nature 389:816 (1997); ii) TRPV4 by phorbol esters: Watanabe et al., J. Biol. Chem. 277:13569 (2002); or iii) changes in physical variables (i.e., for example, temperature or osmolality): Voets et al., Nature 430:748 (2004); and Strotmann et al., Nature Cell Biology 2:695 (2000). Moreover, TRPCs and TRPMs contain a conserved stretch of 25 amino acids called the TRP domain. This is contained in the C-terminus and starts with the nearly invariant EWKFAR (SEQ ID NO:78) TRP box. Montell et al., Cell 108:595 (2002). Using the amino acid pairing computer method discussed herein, it was determined that certain amino acid sequences including, but not limited to, GHLCIRH (SEQ ID NO:79) and EMTPVNPG (SEQ ID NO:80) of GIP matched to TRP calcium channel proteins.

The sequence matches occurred at the TRP signature box and throughout the last 50 amino acids of the TRP carboxyl terminal cytoplasmic tail of the TRP channel protein. Greater increase in slope conductance by linear GIP compared with cyclic GIP is consistent with GIP binding to a regulatory site on the channel protein.

The effect of the reduction of inward current with a reversal potential of zero by substitution of NMDG for external cations indicates that LNCAP cells contain non-selective cation channels that are constitutively open. This is consistent with reports of others, who have established the presence of TRP ion channels in the LNCAP plasma membrane. Vanden Abeele et al., Cell Calcium 33:357 (2003); Sanchez et al., Eur. J. Pharmacol. (In press, 2005).

The fact that consecutive substitution of external cations by NMDG yielded smaller decreases in membrane current may have resulted from volume activation of anionic current (VRAC), which is prevalent in LNCaP cells. Shuba et al., Am. J. Physiol. Cell Physiol. 279:C1144 (2000). The cells might have swollen if they accumulated NMDG by an organic cation transporter along with osmotically obligated water. Nevertheless, whether TRP channels comprise sites of GIP activation requires further study to determine whether the clearly established growth inhibition of LNCaP cells by GIP is attributable to its affects on ion conductance by the plasma membrane. Caceres et al., Anticancer Res. 22:2817 (2002).

C. GIP Subfragments Having Neovacularization Inhibitory Effects

AFP has recently been shown to possess proangiogenic properties that enhance neovascularization and growth in both fetal and tumor tissue. Liang et al., “Oncodevelopmental alpha-fetoprotein acts as a selective proangiogenic factor on endothelial cell from the fetomaternal unit” J Clin Endocrinol Metab 89:1415-1422 (2004); and Takahashi et al., “Angiogenesis of AFP producing gastric carcinoma: Correlation with frequent liver metastasis and its inhibition by anti-AFP antibody” Oncol Rep 11:809-813 (2004). GIP, however, has been shown to inhibit blood vessel formation using a chick chorioallantoic CAM assay and in some tumor models. In one embodiment, the present invention contemplates that GIP-derived peptides inhibit angiogenesis in the tumoral vasculature.

In some embodiments, GIP-derived peptides were identified by homology matching having antiangiogenic regulatory properties comprising:

| | |
|---|---|
| LSEDKLLACGEGAADIIIGHLCIRHEMTPVNPGVG | (SEQ ID NO:2) |
| LSEDKLLACGEGD | (SEQ ID NO:16; linear) |
| DCIRHEMTPVNPGVGD | (SEQ ID NO:19; linear) |
| LSEDKLLE | (SEQ ID NO:23; linear) |
| ECGEGAAD | (SEQ ID NO:24; linear) |
| DIIIGHLCIR | (SEQ ID NO:25; linear) |
| EMTVNPGVGN | (SEQ ID NO:18; linear) |
| LSEDKLLAC............CIRHEMTPVNPGV | (SEQ ID NO:26; linear) |
| LSEDKLLACGEGAADIIIGHLCIRHEMTPVNPGV | (SEQ ID NO:27; P149 cyclic) |
| EAADIIIGHLD | (SEQ ID NO:28; linear) |
| LSEDKLLACGEGAADIIIE | (SEQ ID NO:29; linear) |
| DIGHLCIRHE | (SEQ ID NO:30; linear) |
| TPVNPGVGD | (SEQ ID NO:31; linear) |

A comparative homology analysis allows the construction of the following consensus sequence:

(SEQ ID NO:81)

LSEDKLLX₁CGEX₂X₃ADIX₄IX₅HX₆CIRHEMTPVNPX₇X₈X₉

wherein,

X₁=A or E; X₂=G or E; X₃=A or D; X₄=D or I; X₅=G or E; X₆=L or D;

X₇=V or G; X₈ V or G; and X₉=G or N.

However, in certain embodiments the present invention contemplates an antiangiogenic regulatory consensus sequence having all but one X amino acid substituted with a native GIP amino acid.

D. GIP Subfragments Regulating the Cytoskeletal Structure

GIP-derived peptide fragments and/or subfragments have been assayed for platelet aggregation by measuring light transmissions with an aggregometer using a stirred human platelet suspension. Kinlough-Rathbone et al., In: Platelet Aggregation in Measurements of Platelet Function, Eds. Harker L A, Zimmerman T S., Churchill Livingstone, London. pp. 64-87 (1983); and Whittle et al., “Specificity between the anti-aggregatory actions of prostacyclin, prostaglandin Eland D2 on platelets” In: Mechanisms of stimulus—response coupling in platelets, Eds. V. V. K., MacIntyre D E, Scully M F., New York and London: Plenum Press. pp. 109-115 (1985).

In some embodiments, GIP-derived peptides were identified by homology matching having cytoskeletal modulation properties that control cell shape and size comprising:

| | |
|---|---|
| LSEDKLLACGEGAADIIIGHLCIRHEMTPVNPGVG | (SEQ ID NO:2) |
| EGAADIIIGHLCIRHE | (SEQ ID NO:17; linear) |
| DCIRHEMTPVNPGVGD | (SEQ ID NO:19; linear) |
| LSEDKLLE | (SEQ ID NO:23; linear) |
| KLLACGEGE | (SEQ ID NO:32; linear) |

A comparative homology analysis allows the construction of the following consensus sequence:

LSEDKLLX₁CGEGX₂ADIIIGHX₃CIRHEMTPVNPGV (SEQ ID NO:82), wherein, X₁=A or E; X₂=A or E; and X₃=L or D. However, in certain embodiments the present invention contemplates a cytoskeletal modulation consensus sequence having all but one X amino acid substituted with a native GIP amino acid.

The platelet cytoskeletal-induced response to the activator produces a shape change from the discoid to the spherical form which initiates the aggregation step forming increasingly larger clumps. The GIP has been shown to influence skeletal activation, leading to changes in cell shape and form. Mizejewski et al., “Review and Proposed Action of alpha-fetoprotein growth inhibiting peptides as estrogen and cytoskeletal-associated factors” Internatl. J. Cell Biol. 28:913-933 (2004). The shape change also induces expression of pro-coagulant and phospholipid receptor activity which induces inositol phospholipids to increase the turnover of phosphatidic acid. Kinlough-Rathbone et al., In: Platelet Aggregation in Measurements of Platelet Function, Eds. Harker L A, Zimmerman T S., Churchill Livingstone, London. pp. 64-87 (1983). In the event of a blood vessel rupture, for example, the release of agonists activates blood platelets to manifest the discod-to-spheroid shape change sending out long filopodial extensions from the platelet cytoplasm. The agonists further cause: 1) the centralization of the platelet organelles; 2) release of platelet granules; and 3) formation of a “hemostatic plug” to prevent further blood loss from the blood vessel.

As discussed above, platelet activators (i.e., for example, ADP) induce cell shape changes prior to the primary and secondary platelet aggregation. Chen et al., “Geometric control of cell life and death” Science 276:1425-1428 (1997). Further, GIP-derived peptides are capable of inhibiting the shape change in the platelet response. See FIG. 9 & FIG. 10. Although it is not necessary to understand the mechanism of an invention, it is believed that platelet shape and form is dictated by the submembraneous plasma membrane cytoskeleton comprised mostly of actin/myosin filament components. Fox et al., “Studying the platelet cytoskeleton in Triton X-100 lysates” Methods Enzymol 215:42-58 (1992). ADP induces the change in cell shape by a cytoskeletal-mechanical force-driven redistribution of organelles within the platelet cytoplasm. GIP interaction with the plasma membrane cytoskeleton was demonstrated and exemplified in several models including microtubules and actin filaments. Mizejewski et al., “Review and Proposed Action of alpha-fetoprotein growth inhibiting peptides as estrogen and cytoskeletal-associated factors” Intl Journal Cell Biology (In Press, 2004). The polymerization of actin produces actin filament formation which induces a conformational change in the platelet cell surface fibrinogen receptor (i.e., for example, α_{IIb}β₃). The fibrinogen binding domain is hidden (concealed) on resting platelets and addition of platelet activators (i.e. for example, ADP, arachidonic acid, or collagen) expose such sites. It has been demonstrated that a conformational change in the AFP molecule also exposes the hidden GIP site in a comparable manner. Vakharia et al., “Human alpha-fetoprotein peptides bind estrogen receptor and estradiol, and suppress breast cancer” Breast Cancer Res Treat 63:41-52 (2000).

It was also demonstrated that the GIP was both an estrogen-and cytoskeletal-associated peptide capable of cellular uptake and cytoplasmic (perinuclear) localization. Mizejewski et al., “Review and Proposed Action of alpha-fetoprotein growth inhibiting peptides as estrogen and cytoskeletal-associated factors” Intl Journal Cell Biology (In Press, 2004). It was further reported that the GIP was interactive at the cell surface plasma membrane influencing cellular shape, form, immune agglutination, and peptide aggregation. Since cell shape and form are related to cytoskeletal events and microtubule-associated proteins, these cell surface activities provided clues that the GIP should be capable of modulating cellular adhesion/attachment events. Furthermore, both native AFP and GIP had been previously implicated with erythrocyte agglutination and platelet aggregation as well as tumor cell adhesion to extracellular matrix proteins. Mizejewski G, “Alpha-fetoprotein structure and function: relevance to isoforms, epitopes, and conformational variants” Exp Biol Med 226: 377-408 (2001); Czokalo et al., “Alpha fetoprotein inhibits aggregation of human platelets” Haematologia (Budap) 22:11-18 (1989); Mizejewski G J, “Levels of alpha-fetoprotein in during pregnancy and early infancy in normal and disease states” Obstet Gynecol Surv 58:804-826 (2003); and Brenner et al., “Inhibition by alpha-fetoprotein fractions of hemagglutination reactions between A and B antigens of human red blood cells and specific antisera” Clin Immunol Immunopathol 34:20-26 (1985). The present invention contemplates some embodiments related to the effects of GIP on neoplastic cell growth, adherence, spreading, metastasis and tumor growth arrest by GIP.

E. GIP Subfragments Regulating the Cell Cycle

Levels of α-fetoprotein (AFP) have been shown to correlate with cell cycle regulation regarding tumor growth and proliferation. Lazareva, M N. “Alpha-fetoprotein production by the synchronized regenerating murine liver. Its independence on the phases of the mitotic cycle” Oncodevelopmental Biology & Medicine 2:89-99 (1981); Sasaki et al., “Change of alpha-fetoprotein content during cell cycle of human hepatoma cells in vitro:flow cytometric analysis” Tumour Biology 6:483-490 (1986): and Cao, S L. “Changes in cell cycle of human hepatoma xenograft in nude mice after irradiation” Chinese Journal of Oncology 12:2-14 (1990). AFP is believed to promote both ontogenic and oncogenic growth and has been shown to enhance tumor cell proliferation via protein Kinase A Activity. Wang et al., “Stimulation of tumor-cell growth by alpha-fetoprotein” Int J Cancer 75:596-599 (1998); and Li et al., “The intracellular mechanism of alpha-fetoprotein promoting the proliferation of NIH 3T3 cells” Cell Res 12:151-156 (2002). In contrast, as discussed above, the alpha-fetoprotein (AFP) derived Growth Inhibitory Peptide (GIP) is a synthetic 34-amino acid fragment derived from the full-length native human AFP molecule that suppresses tumor growth. Since the growth suppression by GIP is believed to be cytostatic rather than cytotoxic, one hypothesis suggests that some level of cell cycle arrest might be involved.

Cell cycle transitions are believed orchestrated by cyclin-dependent kinase complexes that consist of a catalytic subunit termed cdk and an activating subunit called cyclin which are paired in diverse combinations. Lees E., “Cyclin dependent kinase regulation” Curr. Opin. Cell Biol. 7:773-780 (1995); and Nigg, E., “The substrates of the cdk2 kinase” Sem. Cell. Biol. 2:262-270 (1991). In mammalian cells, the cyclin E-cdk2 and cyclin D-cdk4 complexes might be catalytically active during G1 phase and rate limiting for cell progression through this stage. Ohtsubo et al., “Human cyclin E, a nuclear protein essential for the G1-to-S phase transition” Mol. Cell. Biol. 15:2612-2624 (1995). An orderly progression through the cycle would be facilitated by activation of different cyclin-cdks at appropriate times. Cyclin-cdks activation is regulated by feedback mechanisms preventing premature entry of cells into the next phase of the cycle prior to completion of the necessary macromolecular events. Bettencourt-Dias et al., “Genome-wide survey of protein kinases required for cell cycle progression” Nature 432:980-987 (2004).

Example 10 investigates the effect of GIP at the transcriptional level of gene expression by determining mRNA levels of selected G1 cell cycle constituents of MCF-7 breast cancer cells. Previous cell proliferation assays had shown that 6-8 days of continuous peptide exposure provided sufficient time for GIP inhibition of MCF-7 cell proliferation to occur. Secondly, Western blots were performed on cell cycle kinase (cdk) inhibitors based on findings from the mRNA determinations by real time-polymerase chain reaction (RT-PCR). Thirdly, both the RT-PCR and Western blot analyses were performed on extracts from MCF-7 cell treated with GIP at low concentrations (10⁻⁸ to 10⁻¹² M) for eight days. Results indicated that G1 specific cell cycle mRNA constituents and kinase inhibitors were indeed modulated by GIP peptide exposure suggesting a cell cycle arrest which may be responsible for GIP-induced growth suppression.

Different members of the cdk family in association with different cyclins turn and/or operate key switches throughout the cell cycle. Cyclin E/cdk 2 complexes operate during the G1 to S phase progression in mammalians cells, and ultimately control events that lead to the initiation of DNA synthesis in the S-phase. Yamada et al., “Loss of cyclin E requirement in cell growth of an oral squamous cell carcinoma cell line implies deregulation of its downstream pathway” International Journal of Cancer 111:17-22 (2004). An increased expression of G1 cyclin proteins has been associated with many tumor types and the presence of abnormally high levels of cyclin E have been observed in human cancers related to reproductive tissues. Payton et al., “Deregulation of cyclin E2 expression and associated kinase activity in primary breast tumors” Oncogene 21:8529-8534; and Groshong et al., “Biphasic regulation of breast cancer cell growth by progesterone: role of the cyclin-dependent kinase inhibitors, p21 and p27^{Kip1}” Molecular Endocrinology 11: 1593-1607 (1997). The cyclin E/cdk2 complexes are required to phosphorylate the retinoblastoma (Rb) protein and drive cells through the G1/S transition into the S-phase of the cell cycle. Thus, the phosphorylation and neutralization of pRb by cdk2 is considered the penultimate step in the transition from the G1 to the S-phase. Fahaeus et al., “Inhibition of pRb phosphorylation and cell-cycle regression by a 20-residue peptide derived from p16^{CDKN2/1NK4A}” Current Biology 6:84-91 (1996). However, such protein cascades usually have backup systems and cdk2 knockout mice have been reported to be viable. Bethet et al., “Cdk2 knockout mice are viable” Current Biology 13:1775-1785 (2003).

The actions of the cdks are regulated by phosphorylation, cyclin levels, the abundance of cdk inhibitors, and their subcellular localization. One mechanism that prevents premature entry in S-phase and ties the G1/S transition to regulatory pathways, relies on inhibitory proteins that dock onto the cyclin/cdk complexes and disrupt their catalytic capability. Russo et al., “Crystal structure of the p27Kip1 cyclin-dependent-kinase inhibitor bound to the cyclin A-Cdk2 complex” Nature 382:325-331 (1996). The p27 Kip1 inhibitor functions as an integral brake of the cell cycle, while the p21 CIP protein serves as a mediator of cytostatic signals. The p21 inhibitors can bind to isolated cdks and their complexes to prevent their activation. However, this binding can also dislodge p27 inhibitor proteins from the complexes and indirectly inhibit cyclin E/cdk2, inducing cell cycle arrest. Toyoshima et al., “p27, a novel inhibitor of G1 cyclin-cdk protein kinase activity, is related to p21” Cell 78:67-74 (1994); Goubin et al., “Identification of binding domains on the p21Cip1 cyclin-dependent kinase inhibitor” Oncogene 10:2281-2287 (1995); Orend et al., “Cytoplasmic displacement of cyclin E-cdk2 inhibitors p21Cip1 and p27Kip1 in anchorage-independent cells” Oncogene 16:2575-2583 (1998); and Adams et al. “Identification of a cyclin-cdk2 recognition motif present in substrates and p21-like cyclin-dependent kinase inhibitors” Molecular and Cellular Biology 16:6623-6633 (1996). The levels of p27 are regulated at the level of translation, and p27 is degraded by ubiquitination and proteosomal involvement following phosphorylation of cyclin E/cdk2 complexes. Alternatively, the INK protein inhibitors (i.e. p15, p16, p18, and p19) can bind to cyclin D/cdk 4,6 complexes and displace bound p27; the displaced p27 can then bind and inhibit cyclin E/cdk2 complexes. Massague J., “G1 cell-cycle control and cancer” Nature 432:298-306 (2004); Scott et al., “Reversible phosphorylation at the C-terminal regulatory domain of p21Waf1/Cip1 modulates proliferating cell nuclear antigen binding” The Journal of Biological Chemistry 275:11529-11537 (2000); Polyak et al., “Cloning of p27Kip1, a cyclin-dependent kinase inhibitor and a potential mediator of extracellular antimitogenic signals” Cells 78:59-66 (1994); and Rempel et al., “Maternal xenopus cdk2-cyclin E complexes function during meiotic and early embryonic cell cycles that lack a G1 phase” Journal of Biological Chemistry 270:6843-6855 (1995).

The data presented in Example 10 confirms that GIP was indeed growth suppressive in MCF-7 cells at the eighth day of cell culture following 80% confluency of the monolayer. Treatment of cyclic GIP for 8 days at 10⁻⁸ to 10⁻¹² M concentrations led to 80 to 90% growth suppression of the MCF-7 cells. The analysis of mRNA expression in the 8-day cell lysates revealed that GIP exposure resulted in a significant down-regulation of Cyclin-E1 mRNA over a peptide concentration range of 10⁻⁸ to 10⁻¹² M. The down-regulated gene expression of Cyclin-E demonstrated reductions ranging from 2.1 to 4.6 fold. These data are noteworthy in that cyclin E levels in the cell are normally 8-fold less than the cdk2 levels. Arooz et al., “On the concentration of cyclins and cyclin-dependent kinases in extracts of cultured human cells” Biochemistry 39:9494-501 (2000).

Lacking sufficient cyclin E as a binding partner, cdk2 should no longer be catalytically active. Concomitantly, GIP treatment had no effect on Ki67, cdk2, cdk6, and Cyclin B1 mRNA levels. In contrast, cyclic GIP exposure resulted in enhanced gene expression as measured by mRNA levels in Cyclin D1 (1.71 fold increase), cdk4 (2.4 fold increase) and Rb1 (2.4 fold increase). Involvement of these G1 cell cycle constituents, especially Cyclin-E1, signified that G1 cell cycle arrest was involved during cell cycle progression.

Western blot determinations presented in Example 10 demonstrate that phosphorylation of p27 was blocked, allowing for persistence of the cdk (Kip/cip)-type inhibitors during the late G1 phase. The continued presence of a nonphosphorylated p27 inhibitor could result in G1 phase cell cycle arrest.

Previous research findings have indicated that decreased levels of p27 (by degradation) may determine breast cancer prognosis and predict a poor outcome for breast cancer patients. Yamada et al., “Loss of cyclin E requirement in cell growth of an oral squamous cell carcinoma cell line implies deregulation of its downstream pathway” International Journal of Cancer 111:17-22 (2004); and Payton et al., “Deregulation of cyclin E2 expression and associated kinase activity in primary breast tumors” Oncogene 21:8529-8534. In one embodiment, the present invention contemplates that GIP-derived peptides may protect or block p27 from phosphorylation and degradation. In one embodiment, p27 levels persist thereby resulting in a G1 cell cycle arrest. Although it is not necessary to understand the mechanism of an invention, it is believed that GIP could function by: i) binding to p27 or p21 complexes; ii) competing as a docking site for cyclin E/cdk complexes; or iii) serving as a decoy phosphorylation substrate to compete for the p21 inhibitor protein. The latter proposed action, the decoy substrate, is believed likely because GIP contains a serine residue on its amino-terminal end, and a threonine residue on its carboxyl-terminal portion. Mizejewski et al., “Review and Proposed Action of alpha-fetoprotein growth inhibiting peptides as estrogen and cytoskeletal-associated factors” Internatl. J. Cell Biol. 28:913-933 (2004). The proposed substrate decoy concept would have to assume that GIP could gain access to the cytoplasmic compartment needed to interact with the Kip/Cip cell cycle inhibitors. Such cellular uptake of GIP by MCF-7 cells has been previously reported. Mizejewski et al., “Alpha-fetoprotein growth inhibitory peptides: potential leads for cancer therapeutics” Mol Cancer Ther 2:1243-1255 (2003).

In some embodiments, GIP-derived peptides were identified by homology matching having properties that control cell cycle activities comprising:

| | |
|---|---|
| LSEDKLLACGEGAADIIIGHLCIRHEMTPVNPGVG | (SEQ ID NO:2) |
| DLLACGEGAADIIIGHLCIRK | (SEQ ID NO:33; linear) |
| DLLACGEGAADIIIGHLCIRK | (SEQ ID NO:34; cyclic) |
| ECGEGAADIIIGHLCIRHEMTPVNPGVD | (SEQ ID NO:35; linear) |
| ECGEGAADIIIGHLCIRHEMTPVNPGVD | (SEQ ID NO:36; cyclic) |
| ECIRHEMTPVNPGD | (SEQ ID NO:37; linear) |
| ETPVNPGD | (SEQ ID NO:38; linear) |

A comparative homology analysis allows the construction of the following consensus sequence:

X₁LLX₂CGEGAADIIIGHX₃CIRX₄EX₅TPVNPX₆X₇ (SEQ ID NO:83), wherein X₁=K or D; X₂=A or E; X₃=L or E; X₄=H or K; X₅=M or E; X₆=V or D; X₇=G or D. However, in certain embodiments the present invention contemplates a cell cycle control consensus sequence having all but one X amino acid substituted with a native GIP amino acid.

Although it is not necessary to understand the mechanism of an invention, it is believed that P149 selectively inhibits tumor cell division without affecting adult normal cells. For example, P149 may modulate some cell cycle regulatory proteins of MCF-7 breast tumor cells. Although it is not necessary to understand the mechanism of an invention, it is believed that P149 arrests MCF-7 cells in the G1 phase of cell division by down regulation of Cyclin E1 mRNA and simultaneously decreases p27 protein phosphorylation. The cell cycle regulatory effect of P149 may be useful to complement current chemotherapy regimens and radiation therapy. Synchronizing the tumor cells and then releasing the population such that they all enter the cell cycle simultaneously allows cytotoxic therapies to be targeted to the time when the cells are most vulnerable and will increase the percent killed.

F. GIP Subfragments Regulating Metabolic Enzymes

In some embodiments, GIP-derived peptides were identified by homology matching having properties that modulate the activity of various metabolic enzymes comprising:

| | |
|---|---|
| LSEDKLLACGEGAADIIIGHLCIRHEMTPVNPGVG | (SEQ ID NO:2) |
| DCIRHEMTPVNPGVGD | (SEQ ID NO:19; linear) |
| KLLACGEGE | (SEQ ID NO:32; linear) |
| DAADIIIGHLCIR | (SEQ ID NO:39; linear) |

A comparative homology analysis allows the construction of the following consensus sequence:

LSEDKLLACGEX₁X₂ADIIIGHX₃CIRHEMTPVNPGVG (SEQ ID NO: 84) wherein, X₁=G or D; X₂=A or E; and X₃=L or D. However, in certain embodiments the present invention contemplates a metabolic enzyme regulation consensus sequence having all but one X amino acid substituted with a native GIP amino acid.

G. GIP Subfragments Regulating Transcription

In some embodiments, GIP-derived peptides were identified by homology matching having properties that modulate the activity of DNA transcription comprising:

| | |
|---|---|
| LSEDKLLACGEGAADIIIGHLCIRHEMTPVNPGVG | (SEQ ID NO:2) |
| LSEDKLLE | (SEQ ID NO:23; linear) |
| ECGEGAAD | (SEQ ID NO:24; linear) |
| DIIIGHLCIR | (SEQ ID NO:25; linear) |
| HEMTPVNPGE | (SEQ ID NO:40; linear) |

A comparative homology analysis allows the construction of the following consensus sequence:

LSEDKLLX₁CGEGAADIIIGHLCIRHEMTPVNPX₂X₃ (SEQ ID NO: 85) wherein, X=A or E; X₂=V or E; and X₃=G or no amino acid. However, in certain embodiments the present invention contemplates a transcription regulation consensus sequence having all but one X amino acid substituted with a native GIP amino acid.

II. GIP Subfragments by Amino Acid Pairing

In some embodiments, the present invention contemplates peptide sequence derivatives using SEQ:ID NO:1 as a template. One method known in the art utilizes an amino acid pairing technique. Root-Bernstein, R. S., “Amino Acid Pairing” J. Theor. Biol. 94:885-859 (1982); and Stefanowicz et al., “The new hypothesis on amino acid complimentarily and its experimental proof” Letters in Peptide Science 5:329-331 (1998). Using the amino acid pairing technique, novel peptide sequences can be derived in approximately 1×10³⁰ combinations and successfully create peptide sequences approximately 30 to 40 amino acids in length. This process usually is followed by both sequence synthesis and functionality screening techniques to validate the predicted sequences. To enhance the probability of success, a methodology was developed that allows for the design of specific peptide sequences that has a high specificity for a putative binding site (i.e., for example, a putative receptor) in combination with a low potential for activating the binding site or receptor.

Although it is not necessary to understand the mechanism of an invention, it is believed that amino acid pairing utilizes the inherent nested sequences in a target peptide (i.e., for example, SEQ ID NO:1) as a template for generating peptide sequences. In one embodiment, GIP-derived peptides may be identified using this method and are believed to have a high binding specificity for regulatory molecules having therapeutic potential. Consequently, the amino acid pairing technique is believed to efficiently develop sequences that offer a high probability of identifying candidate peptide targets tailored for management of specific diseases.

In one embodiment, the present invention contemplates a method for targeting drug candidate development protocol for peptide sequence discovery comprising:
1) Identifying the regulatory proteins involved in the specific disease of interest.
2) Using SEQ ID NO:1 as a template, creating a match algorithm using gene bank sequence mapping similarity/identity tools. This process can also be performed manually. See Table 6 & Table 7. The 26 most probable amino acid pairings are shown in Table 7 according to whether or not they are encoded in the genetic code. The uncoded pairings are derivatives of the coded pairings in which a single base in the triplet has been varied in accordance with the “wobble hypothesis” (Crick, 1966)

3) Maximizing the identity/similarity values for the prospective peptide candidates.

TABLE 6
Amino Acid Codes & Abbreviations

| Code | Amino Acid Name | Abbreviation |
|---|---|---|
| A | Alanine | Ala |
| C | Cysteine | Cys |
| D | Aspartic Acid | Asp |
| E | Glutamic Acid | Glu |
| F | Phenylalanine | Phe |
| G | Glycine | Gly |
| H | Histidine | His |
| I | Isoleucine | Ile |
| K | Lysine | Lys |
| L | Leucine | Leu |
| M | Methionine | Met |
| N | Asparagine | Asn |
| P | Proline | Pro |
| Q | Glutamine | Gln |
| R | Arginine | Arg |
| S | Serine | Ser |
| T | Threonine | Thr |
| V | Valine | Val |
| W | Tryptophan | Trp |
| Y | Tyrosine | Tyr |

TABLE 7
Amino Acid Pairing Table

| | Allows | Nonpolar | Hydrophilic | Radical | Hydrophobic | Stereo- | Charge |
|---|---|---|---|---|---|---|---|
| | backbone | residues | residues | Hydrogen | radical | chemical | transfer |
| | binding | hidden | free | bonding | interaction | fit | complex* |
| Coded | | | | | | | |
| Pairings | | | | | | | |
| Pro-Gly | + | + | | | + | + | |
| Phe-Lys | + | | | | | | + |
| Arg-Ala | + | + | + | ? | | + | |
| Arg-Ser | + | | + | + | | | |
| Ser—Ser | + | | | + | | | |
| Leu-Asn | + | | + | | + | + | |
| Leu-Glu | + | | + | | + | + | |
| Leu-Asp | + | | + | | + | + | |
| His-Val | + | + | + | | + | + | |
| Gln-Val | + | + | + | | + | + | |
| Cys-Thr | + | + | | + | | | |
| Trp-Thr | + | + | + | + | | | |
| Ile-Tyr | + | | | | + | + | |
| Met-Tyr | + | − | | + | + | + | ? |
| Uncoded | | | | | | | |
| Parings | | | | | | | |
| Trp-Ser | + | | | + | | | |
| Thr-Ser | + | − | | + | | | |
| Thr-Arg | + | − | | + | | | |
| Thr—Thr | + | − | | + | | | |
| Phe-Ile | + | + | | | + | + | |
| Trp-Val | + | − | | | + | ? | |
| Trp-Met | + | − | | + | | | |
| Trp-Ile | + | − | + | | + | ? | |
| Tyr-Lys | + | − | | + | | | ? |
| Tyr-Arg | + | | | + | | | ? |
| Phe-Arg | + | | | | | | + |
| His-Ile | + | + | + | | ? | − | |

*Szent-Gyorgyi, “On Energy Transformation” ACTA Phzysiol Acad Sci (Hung) 19: 293-296 (1961)
Positive Interaction (+)
Negative Interaction (−)
Questionable interaction (?)
No interaction (Blank)

In one embodiment, the resulting peptide sequences comprise high binding probabilities for the binding site and/or receptor of interest. In another embodiment, the resulting peptide comprises a low probability of activating any binding site and/or receptor-associated cascade effects. In one embodiment, the identified peptide sequences may be selected from the group comprising templates, lead compounds for the treatment of target disease, or suggest sequence motifs for insertion into SEQ ID NO:1. In one embodiment, the SEQ ID NO:1 may be specifically targeted to a binding site and/or receptor following insertion of a sequence motif.

The GIP-derived peptides described below provide various embodiments of identified sequence motifs. Furthermore, these sequence motifs suggest additional development opportunities for engineered peptides and their associated utility.

A. Structural Motifs of SEQ ID NO:1

1. Sequence Reversal

One embodiment of amino acid pairing contemplates a reverse order amino acid sequence synthesis technique. The Sequence Reversal technique extends to any embodiment that uses partially reversed sequences having a final sequence identity/similarity greater than or equal to 40% as they relate to either the Natural or Reverse sequencing, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a reversed sequence comprising:

| | |
|---|---|
| VGPNVPTMEHRICLHGIIIDAAGEGCALLKDESL. | (SEQ ID NO:41) |

2. D-Amino Acid* Replacement

One embodiment of amino acid pairing contemplates a D-amino acid substitution (i.e., for example, Right Handed amino acid stereoisomers) for each L-amino acid. In one embodiment, the D-amino acid substitution is protease resistant. The Right-Handed Sequence technique extends to any embodiment that uses partially substituted sequences with final sequence identity/similarity greater than or equal to 40% as they relate to either the Natural or Right-Handed sequencing, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a complete D-amino acid sequence. For example:

(SEQ ID NO:42)

L*S*E*D*K*L*L*A*C*G*E*G*A*A*D*I*I*I*G*H*L*C*I*R*-
H*E*M*T*P*V*N*P*G*V*;

In another embodiment, the present invention contemplates a partial D-amino acid sequence. For example:

(SEQ ID NO:143)

L*S*E*D*K*L*L*A*C*G*E*G*AADIIIGHLCIRHEMTPVNPGV;
or

(SEQ ID NO:143)

LSEDKLLACGEGA*A*D*I*I*I*G*H*L*C*I*R*H*E*MTPVNPGV;
or

(SEQ ID NO:144)

LSEDKLLACGEGAADIIIGHLCIRHEM*T*P*V*N*P*G*V*.

3. Coded Amino Acid Pairing

One embodiment of amino acid pairing contemplates genetically coded amino acid substitutions according to Table 7. The Coded Amino Acid Pairing technique extends to any embodiment that uses other potential pairing substitutions, and/or partially substituted sequences with final sequence identity/similarity greater than or equal to 40% as compared to either the Natural or Coded Pairing sequence, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a coded amino acid sequence comprising:

| | |
|---|---|
| DSLLFNERTPLPRRLYYYPINTYSILYWGHLGPQ | (SEQ ID NO:43) |

4. Non-Coded Amino Acid Pairing

One embodiment of amino acid pairing contemplates amino acid substitutions based upon physico-chemical interactions as per Table 7 (i.e., for example, non-coded amino acid pairing). This Non-Coded Amino Acid Pairing technique extends to any embodiment that uses other potential pairing substitutions based on chemical binding affinities, and/or partially substituted sequences with final sequence identity/similarity greater than or equal to 40% as compared to either the Natural or the Non-coded Pairing sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a non-coded amino acid sequence comprising:

| | |
|---|---|
| EWDNYEDRTPLPRRLFWHPINTHTILWSGWLGPQ | (SEQ ID NO:44) |

B. Cargo Bay Motifs of SEQ ID NO:1

1. Nuclear Transcription Motifs

In one embodiment, the present invention contemplates a peptide sequence motif for blocking nuclear transcription by cell membrane/nuclear membrane transport (i.e., NLS-TAT). In addition to this sequence, the Nuclear Transcription technique extends to additional embodiments that use transcription factor peptides sequences known to one having ordinary skill in the art, and/or with such sequences with identity/similarity greater than or equal to 40% as they relate to either the Natural or the Nuclear Transcription sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a nuclear transcription motif comprising YGRKKRRQRRR (SEQ ID NO:45). For example, the motif may be inserted into SEQ ID NO:1 under conditions such that a peptide comprising LYGRKKRRQRRRAADIIIGHLCIRHEMTPVNPGV (SEQ ID NO:46) is created.

2. Decoy Growth Factor Motifs

In one embodiment, the present invention contemplates a peptide sequence motif comprising a decoy growth factor (i.e., for example, a β-Loop epidermal growth factor) sequence. For example, the motif may desensitize growth factor receptors. In addition to this sequence, the Decoy Growth Factor technique extends to additional embodiments using decoy growth factor sequences known to one having ordinary skill in the art, and/or with such sequences with identity/similarity greater than or equal to 40% as they relate to either the Natural or derived Decoy Growth Factor sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a decoy growth factor motif comprising MYIEALDIIKYA (SEQ ID NO:47). For example, the motif may be inserted into SEQ ID NO:1 under conditions such that a peptide comprising LSEDKLLACMYIEALDIIKYACIRHEMTPVNPGV (SEQ ID NO:48) is created.

3. Nuclear Localization Signals and Coadapter Activation Motifs

In one embodiment, the present invention contemplates a peptide sequence comprising a nuclear localization signal motif, and two independent coadapter activation motifs. In one embodiment, the nuclear localization signal motif comprises an amino acid sequence RRKK (SEQ ID NO:49). Although it is not necessary to understand the mechanism of an invention, it is believed that the first motif represents a nuclear localization “zip code” sequence aimed at enhancing nuclear localization. In another embodiment, a firs coadapter motif comprises an amino acid sequence LVQLL (SEQ ID NO: 50). Although it is not necessary to understand the mechanism of an invention, it is believed that the second motif interferes with estrogen receptor activation and may be useful to treat estrogen dependent tumors. In another embodiment, a second coadapter motif comprises an amino acid sequence TTA (SEQ ID NO:51). Although it is not necessary to understand the mechanism of an invention, it is believed that the third motif inhibits receptor phosphorylation. One having ordinary skill in the art should realize that these three independent motifs could be used alone or in any combination with one another.

In addition to the specific sequences exemplified above, an Estrogen Receptor Adapter Sequence technique extends to any additional embodiments that use decoy estrogen receptor adapter sequences known to one having ordinary skill in the art, and/or with such sequences with identity/similarity greater than or equal to 40% as they relate to either the Natural or these derived Steroid Receptor Adapter sequences, including, but not limited to, the SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the three motifs contemplated above may be inserted into SEQ ID NO:1 under conditions such that a peptide comprising LSERRKKRCGEGLVQLLTTALCIRHEMTPVNPGV (SEQ ID NO:52) is created.

4. Transforming Growth Factor Motifs

In one embodiment, the present invention contemplates a peptide sequence motif comprising an epidermal growth factor inhibitor sequence. Although it is not necessary to understand the mechanism of an invention, it is believed that the motif uncouples major tumor growth factor synthesis and/or release. One having ordinary skill in the art would understand that the Transforming Growth Factor Motif technique extends to additional embodiments using transforming growth factor sequences known to one having ordinary skill in the art, and/or with such sequences with identity/similarity greater than or equal to 40% as they relate to either the Natural or derived Transforming Growth Factor sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a transforming growth factor motif comprising VSLTKLLAAGEGCVVGYIGER (SEQ ID NO:53). For example, the motif may be inserted into SEQ ID NO:1 under conditions such that a peptide comprising VSLTKLLAAGEGCVVGYIGERCIRHEMTPVNPGV (SEQ ID NO:54) is created.

5. Apoptosis FAS Motifs

In one embodiment, the present invention contemplates a peptide sequence motif comprising an FAS apoptosis sequence. Although it is not necessary to understand the mechanism of an invention, it is believed that the motif initiates programmed cell death in a cell (i.e., for example, a tumor cell). One having ordinary skill in the art would realize that the Apoptosis FAS technique extends to additional embodiments using other apoptosis sequences known to or discovered by one having ordinary skill in the art, and/or with such sequences with identity/similarity greater than or equal to 40% as they relate to either the Natural or derived apoptosis sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates an apoptosis motif comprising VPIAQKSEP (SEQ ID NO:55). For example, the motif may be inserted into SEQ ID NO:1 under conditions such that a peptide comprising LSEDKLLACGEGAVPIAQKSEPIRHEMTPVNPGV (SEQ ID NO:56) is created.

6. Apoptosis Inhibition Motifs

In one embodiment, the present invention contemplates a peptide sequence comprising an apoptosis inhibition motif and an endoplasmic reticulum release motif. In one embodiment, the present invention contemplates an apoptosis inhibition motif comprising RQGYRVFSLG (SEQ ID NO: 57). Although it is not necessary to understand the mechanism of an invention, it is believed that this first motif blocks a cell (i.e., for example, a tumor cell) for initiating programmed cell death. In another embodiment, the present invention contemplates a an endoplasmic reticulum release motif comprising SL (SEQ ID NO:58). Although it is not necessary to understand the mechanism of an invention, it is believed that this second motif prevents endoplasmic reticulum retention thereby promoting endoplasmic reticulum content release. One having ordinary skill in the art would realize that the first and second motifs could be used independently and/or in combination with each other. Further, the Apoptosis Inhibition technique extends to additional embodiments using other apoptosis sequences known to or discovered by one having ordinary skill in the art, and/or with such sequences with identity/similarity greater than or equal to 40% as they relate to either the Natural or derived apoptosis sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the two motifs contemplated above may be inserted into SEQ ID NO:1 under conditions such that a peptide comprising LSEDKLLACGEGRQGYRVFSLGIRHEMTPVNSLV (SEQ ID NO:59) is created.

7. Viral & Fibroblast Growth Factor Receptor-Actor Competitor Motifs

In one embodiment, the present invention contemplates a peptide sequence comprising a virus-derived motif and a fibroblast growth factor receptor competitor motif. In one embodiment, the present invention contemplates a virus-derived motif comprising RRLKKAAV (SEQ ID NO:60). In one embodiment, the present invention contemplates a fibroblast growth factor receptor competitor motif comprising ALLPAVLLAP (SEQ ID NO:61). Although it is not necessary to understand the mechanism of an invention, it is believed that the combined first and second motifs enhance cell entry and decreases the presence of the fibroblast growth factor receptor in the cell membrane. One having ordinary skill in the art would realize that these motifs could be used alone or in combination with one another. Further, the fibroblast growth factor receptor competitor technique extends to additional embodiments using other apoptosis sequences known to or discovered by one having ordinary skill in the art, and/or with such sequences with identity/similarity greater than or equal to 40% as they relate to either the natural or derived apoptosis sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the two motifs contemplated above may be inserted into SEQ ID NO:1 under conditions such that a peptide comprising LSRRLKKAAVALLPAVLLAPLCIRHEMTPVNPGV (SEQ ID NO:62) is created.

8. Antibacterial Viral Cloaking Motifs

In one embodiment, the present invention contemplates a peptide sequence comprising an antibacterial peptide activator motif, a viral cloaking motif, an apoptosis motif, and a procollagenase enzyme mimic motif. In one embodiment, the present invention contemplates an antibacterial type pore forming motif comprising isoleucine (I: SEQ ID NO: 63). In one embodiment, the present invention contemplates a viral cloaking motif comprising SEDKKLWAS (SEQ ID NO:64). Although it is not necessary to understand the mechanism of an invention, it is believed that this motif could be used to block viral entry and enhance cellular endocytosis. In one embodiment, the present invention contemplates a BH3 apoptosis motif comprising DILRNIAR (SEQ ID NO:65). In one embodiment, the present invention contemplates a procollagenase enzyme mimic motif comprising AQVGDSVGVA (SEQ ID NO:66). Although it is not necessary to understand the mechanism of an invention, it is believed that this motif blocks metastasis by destroying basement membranes. One having ordinary skill in the art would realize that these four motifs could be used independently and/or in combination with each other. Further, the viral cloaking technique extends to additional embodiments using other viral and bacterial cloaking sequences, apoptosis sequences, and antimetastatic sequences known to or discovered by one having ordinary skill in the art, and/or with such sequences with identity/similarity greater than or equal to 40% as they relate to either the Natural or derived sequences, including but not limited to the full length 34-mer peptides, analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the four motifs contemplated above may be inserted into SEQ ID NO:1 under conditions such that a peptide comprising ISEDKKLWASEDILRNIARHLAQVGDSVGVAPGV (SEQ ID NO:67) is created.

9. Chemokine Decoy Ligand Motifs

In one embodiment, the present invention contemplates a peptide sequence comprising a chemokine decoy receptor motif. Although it is not necessary to understand the mechanism of an invention, it is believed that this motif binds to a specific portion of the chemokine receptor in a manner that does not activate the receptor. Further, the technique extends to additional embodiments using other chemokine decoy receptor sequence pairings known to or discovered by one having ordinary skill in the art, and/or with such sequences with identity/similarity greater than or equal to 40% as they relate to either the Natural or derived sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a chemokine decoy receptor motif comprising ASDLAIDLYHIRT (SEQ ID NO: 68). For example, the motif may be inserted into SEQ ID NO:1 under conditions such that a peptide comprising LSEDKLLACGEGASDLAIDLYHIRTEMTPVNPGV (SEQ ID NO:69) is created.

10. Chemokine CXCR4 Decoy Receptor Motifs

In one embodiment, the present invention contemplates a peptide sequence comprising a CXCR4 decoy soluble receptor motif. Although it is not necessary to understand the mechanism of an invention, it is believed that this motif, when used as a decoy soluble receptor, competitively binds to circulating chemokine ligands (i.e., for example, SDF-1). Further, the technique extends to additional embodiments using other chemokine decoy soluble receptor sequences known to or discovered by one having ordinary skill in the art, and/or with such sequences with identity/similarity greater than or equal to 40% as they relate to either the natural or derived sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a chemokine CXCR4 decoy receptor motif comprising ISLDRYLAIVHAT (SEQ ID NO: 70). For example, the motif may be inserted into SEQ ID NO:1 under conditions such that a peptide comprising LSEDKLLACGEGISLDRYLAIVHATEMTPVNPGV (SEQ ID NO:71) is created.

11. Soluble D6 Chemokine Receptor Motifs

In one embodiment, the present invention contemplates a peptide sequence comprising a D6 Duffy chemokine decoy soluble receptor. Although it is not necessary to understand the mechanism of an invention, it is believed that the motif will competitively bind multiple circulating chemokine ligands. Note that the only difference between this motif and a CXCR4 receptor motif is a E²⁰→A²⁰ substitution. Further, the technique extends to additional embodiments using other chemokine decoy soluble receptor sequences known to, or discovered by, one having ordinary skill in the art, and/or with such sequences with identity/similarity greater than or equal to 40% as they relate to either the natural or derived sequences, including, but not limited to, SEQ ID NO:1, its analogs, derivatives, conjugates, or multimer configurations (dimers, trimers, oligomers), conformational changes (linear, cyclic, etc.) and any and all partial fragments and/or their combinations that share identity/similarities equal to or greater than 40%. In one embodiment, the present invention contemplates a chemokine D6 decoy receptor motif comprising ISLDRYLEIVHAT (SEQ ID NO: 72). For example, the motif may be inserted into SEQ ID NO:1 under conditions such that a peptide comprising LSEDKLLACGEGISLDRYLEIVHATEMTPVNPGV (SEQ ID NO:73) is created.

III. Protease Resistance

In one embodiment, the present invention contemplates GIP-derived peptides that are protease resistant. In one embodiment, such protease-resistant peptides are peptides comprising protecting groups. In one embodiment, endoprotease-resistance is achieved using peptides which comprise at least one D-amino acid. In another embodiment, exoprotease-resistance is achieved using peptides which comprise at least one terminal protecting group. In one embodiment, endoprotease-resistance is achieved using a terminally protected GIP-derived peptide.

In another embodiment, the present invention contemplates a GIP-derived peptide that is protected from exoprotease degradation by N-terminal acetylation (“Ac”) and C-terminal amidation. A protected GIP-derived peptide is useful for in vivo administration because of its resistance to proteolysis.

In another embodiment, the present invention also contemplates peptides protected from endoprotease degradation by the substitution of L-amino acids in said peptides with their corresponding D-isomers. It is not intended that the present invention be limited to particular amino acids and particular D-isomers. This embodiment is feasible for all amino acids, except glycine; that is to say, it is feasible for all amino acids that have two stereoisomeric forms. By convention these mirror-image structures are called the D and L forms of the amino acid. These forms cannot be interconverted without breaking a chemical bond. With rare exceptions, only the L forms of amino acids are found in naturally occurring proteins.

Although it is not necessary to understand the mechanism of an invention, it is believed that for short peptide fragments or subfragments, a terminal protecting group has sufficient steric hindrance such that the protecting group is effective against endoprotease and exoprotease activities. Such terminal protecting groups may be attached to either the N-terminal, C-terminal peptide ends, or both. Such terminal protecting groups include, but are not limited to, an acyl, an amide, an acetate, a benzyl, or a benzoyl group. It is further believed that terminally protected GIP protein fragments having between 4 and 40 amino acids, preferably between 8 and 20 amino acids, but more preferably between 10 and 15 amino acids have improved biological half-lives and bioavailability.

### EXPERIMENTAL

### Example 1

### Peptide Synthesis, Characterization, and Properties

Peptides for all experiments were synthesized by F-MOC chemistry using an Applied Biosystems 431A peptide synthesizer (Foster City, Calif.), and were purified using reverse phase high pressure liquid chromatography.

Smaller peptide fragments of the 34-mer P149 GIP peptide were also synthesized for experimental studies. The amino terminal sequence of twelve amino acids was designated P149a. The middle sequence of fourteen amino acids was designated P149b, and the carboxy terminal segment of eight amino acids was designated P149c. A cyclic version of the 34-mer P149 was synthesized with a disulfide bond created by the two cysteines.

Several peptides were created to serve as experimental controls. A mutated form of the 34-mer P149 was created by changing aspartic acid at position 4 to aspargine was designated P187. A scrambled version of P149, designated P263, and an albumin-derived peptide fragment with some sequence homology to P149 completed the set of control peptides.

Biochemical and biophysical studies of the 34-mer P149 demonstrated a molecular mass of 3573 Daltons determined by electrospray ionization mass spectroscopy. The far UV circular dichroism displayed a negative maximum at 201 nm and indicated the presence of β-sheets (45%) and other ordered structures in equal proportions (45%). The remaining structures were composed of a-helix forms (10%). Both Fourier-infrared spectroscopy and GCG computer modeling software confirmed the presence of a largely β-sheet structure.

GIP-derived sequences may also be obtained or simulated by smaller peptide segment constructs, using basic chemistry methods other than amino acid binding, known to one having ordinary skill in the art, including but not limited, to conjugation, polymerization, nanotechnology, etc.

Similarly effective and/or enhanced peptide sequences may also be constructed via amino acid substitution, addition, and/or omission methods using technologies known to one having ordinary skill in the art, including but not limited to, the substitutions found in Table 8. Omissions may include single or multiple amino acid omissions within a specified amino acid sequence, or groups of omitted sequences as they may be found in natural folded or un-folded states of the parent protein.

TABLE 8
GIP-derived Parent And Daughter Sequences.

| | | |
|---|---|---|
| GIP-L36 (Linear GIP, 36-mer) | NH₂-Leu-Ser-Glu-Asp-Lys-Leu-Leu-Ala-Cys-Gly-Glu- | (SEQ ID NO: 129) |
| (Methylated Cysteines at | Gly-Ala-Ala-Asp-Ile-Ile-Ile-Gly-His-Leu-Cys-Ile- | |
| Cys⁹ & Cys²²) | Arg-His-Glu-Met-Thr-Pro-Val-Asn-Pro-Gly-Val-Gly- | |
| | Gln-COOH | |
| GIP-C36 (Cyclic GIP, 36-mer) | NH₂-Leu-Ser-Glu-Asp-Lys-Leu-Leu-Ala-[Cys-Gly-Glu- | (SEQ ID NO: 130) |
| (Disulfide Bonds between | Gly-Ala-Ala-Asp-Ile-Ile-Ile-Gly-His-Leu-Cys]-Ile- | |
| Cys⁹ & Cys²²) | Arg-His-Glu-Met-Thr-Pro-Val-Asn-Pro-Gly-Val-Gly- | |
| | Gln-COOH | |
| GIP-L37 (Linear GIP, 37-mer) | NH₂-Gln-Leu-Ser-Glu-Asp-Lys-Leu-Leu-Ala-Cys-Gly- | (SEQ ID NO: 131) |
| (Methylated Cysteines at | Glu-Gly-Ala-Ala-Asp-Ile-Ile-Ile-Gly-His-Leu-Cys- | |
| Cys¹⁰ & Cys²³) | Ile-Arg-His-Glu-Met-Thr-Pro-Val-Asn-Pro-Gly-Val- | |
| | Gly-Gln-COOH | |
| GIP-C37 (Cyclic GIP, 37-mer) | NH₂-Gln-Leu-Ser-Glu-Asp-Lys-Leu-Leu-Ala-[Cys-Gly- | (SEQ ID NO: 132) |
| (Disulfide Bonds between | Glu-Gly-Ala-Ala-Asp-Ile-Ile-Ile-Gly-His-Leu-Cys]- | |
| Cys¹⁰ & Cys²³) | Ile-Arg-His-Glu-Met-Thr-Pro-Val-Asn-Pro-Gly-Val- | |
| | GLy-Gln-COOH | |
| GIP-2C39 (Bi-Cyclized GIP, 39-mer) | [Cys-Gln-Leu-Ser-Glu-Asp-Lys-Leu-Leu-Ala-Cys]-Gly- | (SEQ ID NO: 133) |
| (Disulfide Bonds between Cys¹-Cys¹¹ | Glu-Gly-Ala-Ala-Asp-Ile-Ile-Ile-Gly-His-Leu-[Cys- | |
| & Cys²⁴-Cys³⁹) | Ile-Arg-His-Glu-Met-Thr-Pro-Val-Asn-Pro-Gly-Val- | |
| | Gly-Gln-Cys] | |
| GIP-2C41 (Bi-Cyclized GIP, 41-mer) | [Cys-Cys-Gln-Leu-Ser-Glu-Asp-Lys-Leu-Leu-Ala-Cys]- | (SEQ ID NO: 134) |
| (Disulfide Bonds between Cys¹-Cys¹² | Gly-Glu-Gly-Ala-Ala-Asp-Ile-Ile-Ile-Gly-His-Leu- | |
| & Cys²⁵-Cys⁴¹) | [Cys-Ile-Arg-His-Glu-Met-Thr-Pro-Val-Asn-Pro-Gly- | |
| | Val-Gly-Gln-Cys-Cys] | |
| GIP-3C41 (Tri-Cyclized GIP, 41-mer) | [Cys-Gln-Leu-Ser-Glu-Asp-Lys-Leu-Leu-Ala-Cys]- | (SEQ ID NO: 135) |
| (Disulfide Bonds between Cys¹-Cys¹¹ | [Cys-Gly-Glu-Gly-Ala-Ala-Asp-Ile-Ile-Ile-Gly-His- | |
| & Cys¹²-Cys²⁵ & Cys²⁶-Cys⁴¹) | Leu-Cys]-[Cys-Ile-Arg-His-Glu-Met-Thr-Pro-Val-Asn- | |
| | Pro-Gly-Val-Gly-Gln-Cys] | |
| GIP-FPL9 (Linear GIP Front Piece, | Gln-Leu-Ser-Glu-Asp-Lys-Leu-Leu-Ala | (SEQ ID NO: 136) |
| 9-mer) | | |
| GIP-MPL12 (Linear GIP Mid Piece, | Gly-Glu-Gly-Ala-Ala-Asp-Ile-Ile-Ile-Gly-His-Leu | (SEQ ID NO: 136) |
| 12-mer) | | |
| GIP-BPL14 (Linear GIP Back Piece, | Ile-Arg-His-Glu-Met-Thr-Pro-Val-Asn-Pro-Gly-Val- | (SEQ ID NO: 137) |
| 14-mer) | Gly-Gln | |
| GIP-FPC11 (Cyclic GIP Front Piece, | [Cys-Gln-Leu-Ser-Glu-Asp-Lys-Leu-Leu-Ala-Cys] | (SEQ ID NO: 138) |
| 11-mer) | | |
| (Disulfide Bonds between Cys¹-Cys¹¹) | | |
| GIP-MPC14 (Cyclic GIP Mid Piece, | [Cys-Gly-Glu-Gly-Ala-Ala-Asp-Ile-Ile-Ile-Gly-His- | (SEQ ID NO: 139) |
| 14-mer) | Leu-Cys] | |
| (Disulfide Bonds between Cys¹-Cys¹⁴) | | |
| GIP-BPC16 (Cyclic GIP Back Piece, | [Cys-Ile-Arg-His-Glu-Met-Thr-Pro-Val-Asn-Pro-Gly- | (SEQ ID NO: 140) |
| 16-mer) | Val-Gly-Gln-Cys] | |
| (Disulfide Bonds between Cys¹-Cys¹⁶) | | |

Similarly effective and/or enhanced peptide sequences may also be constructed using a variety of cyclization, glyco-silation, and/or other stabilization or uptake enhancement techniques known to one having ordinary skill in the art.

Larger peptide conjugates may also be constructed using any one or more of the three front, mid or back sub-peptides described above (i.e., for example, P149a, P149b, or P149c). These larger peptide conjugates may be made in any combination or with any number of constituent GIP-derived peptides. These peptides may also be constructed using a variety of additional cyclization, glyco-silation, and/or other stabilization or uptake enhancement techniques known to one having ordinary skill in the art.

It is known that naturally-occurring amino acids (left side) and other amino acids that can functionally substitute for each one. See Table 9. Such properties depend on their physico/chemical structure similarities such as A) nonaromatic versus aromatic; B) polar versus non-polar; C) polar uncharged versus polar charged; D) ionizable degree versus less ionizable degree; and E) special structural properties.

TABLE 9
Amino Acid Substitutions

| Amino Acids | Suitable Substitute Amino Acids |
|---|---|
| A - Alanine (ALA) | Val (V), Leu (L), Ile (I), SER (S), THR (T), Asn (N), Gln (Q) |
| C - Cysteine (Cys) | Met (M), Pro (P), Val (V), Leu (L), Ile (I) |
| D - Aspartic Acid (ASP) | Glu (E), GLN (Q), ASN (N) |
| E - Glutamic Acid (Glu) | ASP (D), GLN (Q), ASN (N) |
| F - Phenylalanine (PHe) | Val (V), Leu (L), ILe (I), TRp (W), Pro (P) |
| G - Glycine (GLY) | Ser (S), THR (T), ASN (N), GLN (Q) |
| H - Histidine (His) | Lys (K), Arg (R) |
| I - Isoleucine (ILe) | Val (V), Leu (L), ILe (I), PHe (F), TRP (W), Cys (C) |
| K - Lysine (LYS) | Arg (R), His (H) |
| L - Leucine (LEU) | Val (V), Leu (L), ILe (I), PHe (F), TRP (W), Cys (C) |
| M - Methionine (MET) | Val (V), Leu (L), ILe (I), PHe (F), TRP (W), Cys (C) |
| N - Asparagine (ASN) | GLN (Q), THR (T), SER (S), GLY (G), ASP (D), GLU (E) |
| P - Proline (PRO) | His (H), Met (M), Cys (C), PHe (F), TRP (W) |
| Q - Glutamine (GLN) | GLN (Q), THR (T), SER (S), GLY (G), ASP (D), GLU (E) |
| R - Arginine (Arg) | Lys (K), Arg (R) |
| S - Serine (SER) | GLY (G); THR (T), ASN (N), GLN (Q), Cys (C) |
| T - Threonine (THR) | GLY (G), SER (S), ASN (N), GLN (Q), Cys (C) |
| V - Valine (Val) | PHe (F), Trp (W), Cys (C), Ile (I), Leu (L), Val (V) |
| W - Tryptophan (Trp) | Phe (F), His (H), TRP (W), Leu (L), Ile (I), Val (V) |
| Y - Tyrosine (Tyr) | Phe (F), Trp (W), Cys (C), Ile (I), Leu (L), Val (V) |

### Example 2

### GIP-Induced Modulation of Cell Cycle Gene mRNA Expression

Overview

GIP, in both its linear and cyclic forms, can regulate gene expression in the breast cancer cell line, MCF-7. Using quantitative reverse transcriptase polymerase chain reaction (Quantitative RT-PCR) GIP-induced changes in the mRNA levels of several genes involved in cell cycle control were determined.

A preliminary screen of mRNA after 24 or 48 hours of exposure to GIP revealed little differences in expression levels of any of the six genes investigated {CDK2, CDK4, CDK6, Cyclin D 1, CCND3, or RB 1). During the second stage of screening, however, MCF-7 cell cultures were exposed to various concentrations of the linear peptide for 24, 48, or 72 hours. Further, the MCF-7 cell cultures were exposed to the cyclic peptide for eight days. The eight-day exposure to the cyclic peptide revealed significant changes in mRNA levels in several of the genes examined. Of particular interest are Cyclin E and RB 1.

MCF-7 cell cultures grown in charcoal-stripped medium for 24 hours were seeded in 6-well plates and allowed to attach for 24 hours before treatment with 1×10⁻⁷M of the cyclic, linear, or scrambled peptide, or a vehicle control for either 24 or 48 hours. Total RNA was isolated using TriReagent (Molecular Research Center Inc.) and quantified using the Genequant Pro (Ambersham Biosciences).

TABLE 10
RNA isolated from MCF-7 cell cultures after treatment for either
24 or 48 hours.

| | | 260/280 |
|---|---|---|
| | Total μg | Ratio |
| 24 Hours | | |
| Linear | 14.32 | 1.97 |
| Cyclic | 14.28 | 1.89 |
| Scrambled | 12.43 | 1.85 |
| Control | 18.53 | 1.93 |
| 48 Hours | | |
| Linear | 15.60 | 1.97 |
| Cyclic | 12.60 | 1.95 |
| Scrambled | 19.96 | 1.83 |
| Control | 13.72 | 1.92 |

Table 10 (supra) shows the quantity and quality of RNA isolated from the first set of eight treatments. As can be seen, the quality of the RNA was extremely high. Primers for each of six genes were designed using Primer 3 and purchased from Integrated DNA Technologies. Table 11.

TABLE 11
Primer Sequences

| | | |
|---|---|---|
| Gene Name | Forward Primer | Reverse Primer |

| | | | | |
|---|---|---|---|---|
| Cyclin D3 | 5′-GTGGCCACTAAGCAGAGGAG-3′ | (SEQ ID NO:109) | 5′-AGCTTGACTAGCCACCGAAAA-3′ | (SEQ ID NO:110) |
| (CCND3) | | | | |
| Cyclin D1 | 5′-GAGGAAGAGGAGGAGGAGGA-3′ | (SEQ ID NO:111) | 5′-GAGATGGAAGGGGGAAAGAG-3′ | (SEQ ID NO:112) |
| CDK2 | 5′-CATTCCTCTTCCCCTCATCA-3′ | (SEQ ID NO:113) | 5′-CAGGGACTCCAAAAGCTCTG-3′ | (SEQ ID NO:114) |
| CDK4 | 5′-GAAACTCTGAAGCCGACCAG-3′ | (SEQ ID NO:115) | 5′-AGGCAGAGATTCGCTTGTGT-3′ | (SEQ ID NO:116) |
| CDK6 | 5′-AGAGACAGGAGTGGCCTTGA-3′ | (SEQ ID NO:117) | 5′-TGAAAGCAAGCAAACAGGTG-3′ | (SEQ ID NO:118) |
| RB1 | 5′-GGAAGCAACCCTCCTAAACC-3′ | (SEQ ID NO:119) | 5′-TTTCTGCTTTTGCATTCGTG-3′ | (SEQ ID NO:120) |
| Ki67 | 5′-AGTCAGACCCAGTGGACACC-3′ | (SEQ ID NO:121) | 5′-TGCTGCCGGTTAAGTTCTCT-3′ | (SEQ ID NO:122) |
| Cyclin E1 | 5′-CAGATTGCAGACCTGTTGGA-3′ | (SEQ ID NO:123) | 5′-TCCCCGTCTCCCTTATAACC-3′ | (SEQ ID NO:124) |
| Cyclin B1 | 5′-CGGGAAGTCACTGGAAACAT-3′ | (SEQ ID NO:125) | 5′-AAACATGGCAGTGACACCAA-3′ | (SEQ ID NO:126) |
| β-Actin | 5′-GGACTTCGAGCAAGAGATGG-3′ | (SEQ ID NO:127) | 5′-AGCACTGTGTTGGCGTACAG-3′ | (SEQ ID NO:128) |

Standard curves for each primer were prepared and RNA samples were reverse transcribed and screened for the genes of interest using the One Step RT-PCR kit (Qiagen Inc.) in the Roche Light Cycler. Each sample was run in duplicate, and the entire experiment was replicated resulting in two independent samples for each treatment. The combined data from the two experiments are shown in FIG. 13. In general, treatment with either the linear or cyclic peptide for 24 or 48 hours did not result in significant changes in mRNA levels of the selected genes. One possible exception is the expression of CDK2; treatment with 1×10⁻⁷ M of the linear peptide for 48 hours appeared to decrease the mRNA levels of CDK2.

### Example 3

### Dose Response Analysis of Linear GIP-Induced Gene Expression

MCF-7 cell cultures were maintained in medium with charcoal-stripped bovine serum for 24 hours prior to seeding in 6-well plates. Seeding densities varied with treatment time and were aimed at obtaining 80% confluent cell cultures at the time of harvesting; after 24, 48, or 72 hours of treatment. RNA isolation was performed as above.

TABLE 12
RNA isolated from MCF-7 cell cultures exposed to various concentrations
of the linear form of GIF or 24, 48, or 72 hours.

| | Concentration (M) | Total μg | 260/280 Ratio |
|---|---|---|---|
| 24 Hours | Control | 10.23 | 1.78 |
| | 1e−8 | 13.45 | 1.84 |
| | 1e−7 | 11.08 | 1.84 |
| | 1e−6 | 15.04 | 1.89 |
| | 1e−5 | 13.45 | 1.85 |
| 48 Hours | Control | 13.20 | 1.90 |
| | 1e−8 | 14.72 | 1.85 |
| | 1e−7 | 15.23 | 1.79 |
| | 1e−6 | 12.43 | 1.96 |
| | 1e−5 | 13.22 | 1.86 |
| 72 Hours | Control | 16.94 | 1.92 |
| | 1e−8 | 16.86 | 1.85 |
| | 1e−7 | 19.34 | 1.78 |
| | 1e−6 | 15.35 | 1.88 |
| | 1e−5 | 15.31 | 1.81 |

As shown in Table 12 (supra), high quality RNA was recovered from each of the treatment; four concentrations plus a vehicle control for each of the time points. The mRNA expression levels of four genes, Cyclin B1, CDK2, Ki67, and Cyclin E1, were determined using Quantitative RT-PCR. RNA samples were run in duplicate. See FIG. 14. As in the preliminary screen, treatment with the linear peptide induced few clear changes in gene expression.

### Example 4

### Dose Response Analysis of Cyclic GIP-Induced Gene Expression

MCF-7 cell cultures were maintained as above and seeded in 6-well plates at a low seeding density to obtain cell cultures that were 80% confluent at the end of the treatment. Cell cultures were treated with various concentrations of the cyclic peptide 24 hours after seeding and then every two days until harvesting the cells at day eight post-treatment RNA was isolated from each treatment.

TABLE 13
RNA isolated from MCF-7 cell cultures exposed to the cyclic form of
GIF for eight days.

| Concentration (M) | Total μg | 260/280 Ratio |
|---|---|---|
| Control | 17.24 | 2.047 |
| 1e−12 | 16.30 | 1.814 |
| 1e−11 | 16.74 | 1.925 |
| 1e−10 | 15.98 | 1.910 |
| 1e−09 | 17.46 | 1.925 |

As shown in Table 13 (supra), high quality RNA was obtained from each of the treatments. mRNA expression levels of eight genes; Cyclin E1, Cyclin B1, Cyclin D1, RBI, Ki67, CDK2, CDK4, and CDK6, were tested in duplicate samples using quantitative RT-PCR. Results on expression levels are shown in FIGS. 15 and 16. Four of the genes (Ki67, CDK2, CDK6, and Cyclin B1) show little changes in mRNA expression levels at any of the concentrations tested. The expression of three genes, Cyclin DI, RBI, and CDK4, is increased about two-fold with treatment of 0.1 nM (Cyclin DI and RB1) or 1 pM (CDK4) of the cyclic peptide. The expression levels of Cyclin E1 are greatly reduced at each of the five concentrations tested. See Table 14.

TABLE 14
Cyclin E1 mRNA levels after an eight day treatment with the cyclic form
of GIP.

| Concentration | Fold Increase | Fold Decrease |
|---|---|---|
| Control | 0.00 | 0.00 |
| 1e−12 | − | 3.26 |
| 1e−11 | − | 2.36 |
| 1e−10 | − | 2.07 |
| 1e−09 | − | 3.03 |
| 1e−08 | − | 4.58 |

Interestingly, the reduction observed at 1 pM (3.26 fold) is nearly as large as the reduction observed at 10 nM (4.58 fold).

Clearly, the data show that prolonged treatment with the cyclic peptide, at concentrations ranging from 1 pM to 10 nM, greatly reduces the mRNA levels of Cyclin E1. Cyclin E activity is one of the major factors determining whether cells will proceed through cell cycle cascade or growth arrest and return to G0. In early to mid G1, when mitogenic signals begin to accumulate, Cyclin D-CDK4/6 complexes begin to phosphorylate pRB and to sequester p21 Cip and p27Cip molecules. At the same time, E2F is being released by the partially phosphorylated pRB inducing Cyclin E/CDK2. The Cyclin E complexes then induce the hyperphosphorylation of pRB fully inactivating it. The Cyclin E complexes are also capable of inducing p27 degradation by phosphorylating the Thr-187 site on p27, targeting the molecule for ubiquitination-mediated proteolysis. The down-regulation of Cyclin E1 suggests that the cyclic GIP may inhibit cell growth by disruption of the cell cycle.

### Example 5

### Extended Dose Response Analysis of Cyclic GIP-Induced Gene Expression

MCF-7 cells were seeded in DC5 medium into each well of two 6-well plates and allowed to attach for 24 hours at which point the seeding medium was exchanged for serum-free medium for 24 hours. After 24 hours in serum-free medium the cells were re-fed with DC5-stripped medium containing either the vehicle control (DMSO) or GIP (from 1×10⁻¹⁴ to 1×10⁻⁵ M). Cells were re-fed every 48 hours until they were harvested after eight days of treatment and RNA was isolated. The entire procedure was repeated resulting in two independent RNA isolations. The quantity and quality of RNA isolated from each eight-day exposure for each of the concentrations are shown below. See Table 15. The quantity and quality of RNA obtained from these exposures is sufficient to determine the RNA expression levels of numerous genes.

TABLE 15
MCF-7 Cell Culture RNA Quality And Quantity

| | Isolation One | | Isolation Two | |
|---|---|---|---|---|
| Concentration of GIP (Molarity) | Total μg | 260/280 | Total μg | 260/280 |
| Control 1 | 8.22 | 1.63 | 22.32 | 1.75 |
| Control 2 | 11.06 | 1.64 | 10.76 | 1.67 |
| 1 × 10⁻¹⁴ | 9.7 | 1.67 | 38.62 | 1.82 |
| 1 × 10⁻¹³ | 11.7 | 1.66 | 18.34 | 1.69 |
| 1 × 10⁻¹² | 19.32 | 1.50 | 15.5 | 1.66 |
| 1 × 10⁻¹¹ | 17.22 | 1.44 | 22.46 | 1.72 |
| 1 × 10⁻¹⁰ | 16.22 | 1.41 | 13.06 | 1.70 |
| 1 × 10⁻⁹ | 14.22 | 1.60 | 21.38 | 1.55 |
| 1 × 10⁻⁸ | 17.36 | 1.60 | 13.66 | 1.43 |
| 1 × 10⁻⁷ | 20.16 | 1.65 | 17.72 | 1.61 |
| 1 × 10⁻⁶ | 18.86 | 1.55 | 24.38 | 1.61 |
| 1 × 10⁻⁵ | 11.62 | 1.50 | 21.66 | 1.52 |

Examination of the levels of Cyclin E1 transcript provides insight into cell cycle control. In Example 4, treatment with 1×10⁻¹² M GIP for eight days resulted in decreased expression of Cyclin E1. This example presents exemplary data examining GIP low concentrations on Cyclin E1 transcript expression. GIP was shown to have no effect at 1×10⁻¹⁴ M and induces maximal inhibition of Cyclin E1 transcript expression at 1×10⁻¹² M. See FIG. 17. The quantitative RT-PCR data were obtained with the Roche Light Cycler using RNA from Isolation One, run three separate times. See FIG. 18.

### Example 6

### Mouse Ascites Mammary Tumor Assay

The growth suppression of the mammary ascites tumor by AFP peptides and its fragments P149a, P149b and P149c, and control peptides were determined in non-estrogen dependent assays which measured tumor cell growth and ascites accumulation of 6WI-1 mammary transplanted NYLAR/nya mice. Vakharia et al., “Human alpha-fetoprotein peptides bind estrogen receptor and estradiol, and suppress breast cancer” Breast Cancer Res Treat 63:41-52 (2000); and Mizejewski et al., “Alpha-fetoprotein growth inhibitory peptides: potential leads for cancer therapeutics” Mol Cancer Ther 2:1243-1255 (2003). Tumor cell inocula included doses of 0.3, 1.0, and 3.0×10⁶ cells which produced host mortality at 12-14 days which was lethal in 100% of the animals. Following peptide dose titration studies, mice inoculated with 6WI-1 mammary cells were injected with a previously-determined optimal dose of 1.0 μg peptide per day or saline for 11 days. On day 12 following inoculation, the total accumulated ascites fluid volume and tumor cells in the peritoneal cavity of each animal were harvested and the cell count determined. Vakharia et al., “Human alpha-fetoprotein peptides bind estrogen receptor and estradiol, and suppress breast cancer” Breast Cancer Res Treat 63:41-52 (2000).

Inoculation of mice with all three cell doses led to an increase in body weight from 25 g (day 0) to approximately 4S g by day 12, due to the accumulation of tumor cells and ascites fluid in the intraperitoneal cavity. An average cell count in accumulated ascites fluid on day 12 was greater than 10⁸ cells/ml with a total ascites volume approximating 20 mls. Tumor cell proliferation in this model has been reported to correlate with the volume of ascites fluid accumulated. Vakharia et al., “Human alpha-fetoprotein peptides bind estrogen receptor and estradiol, and suppress breast cancer” Breast Cancer Res Treat 63:41-52 (2000); and Mizejewski et al., “Alpha-fetoprotein growth inhibitory peptides: potential leads for cancer therapeutics” Mol Cancer Ther 2:1243-1255 (2003). It was observed that GIP, at 1 μg/day for 11 days, suppressed the tumor-associated body weight gain (significant p<0.05) at the two higher cell doses and totally suppressed body weight increase at the lowest cell dose.

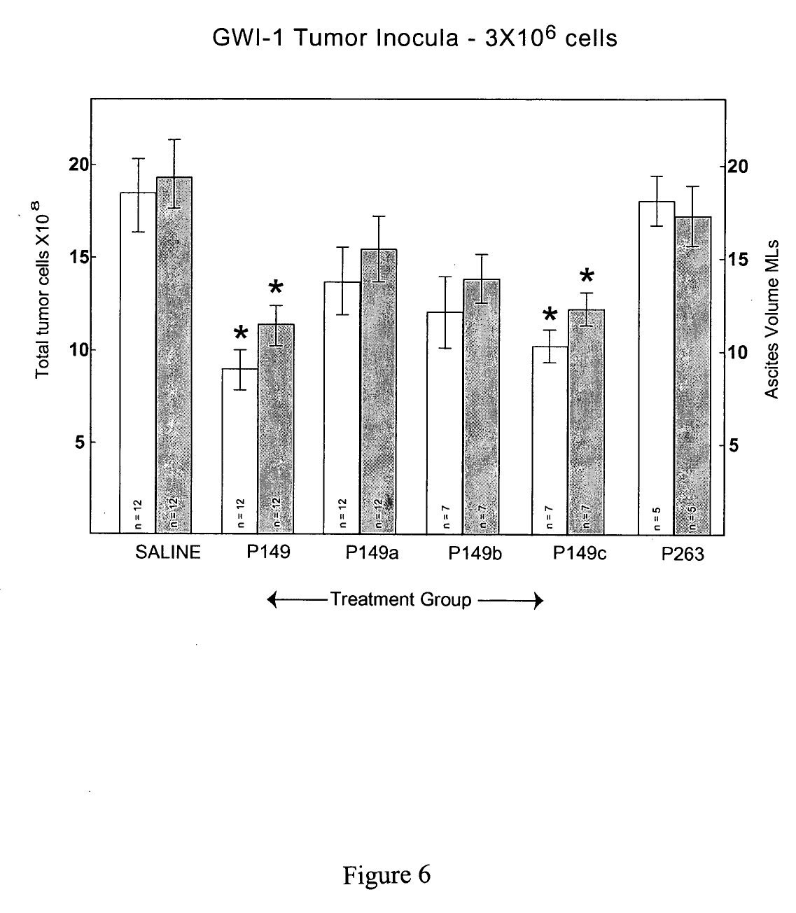

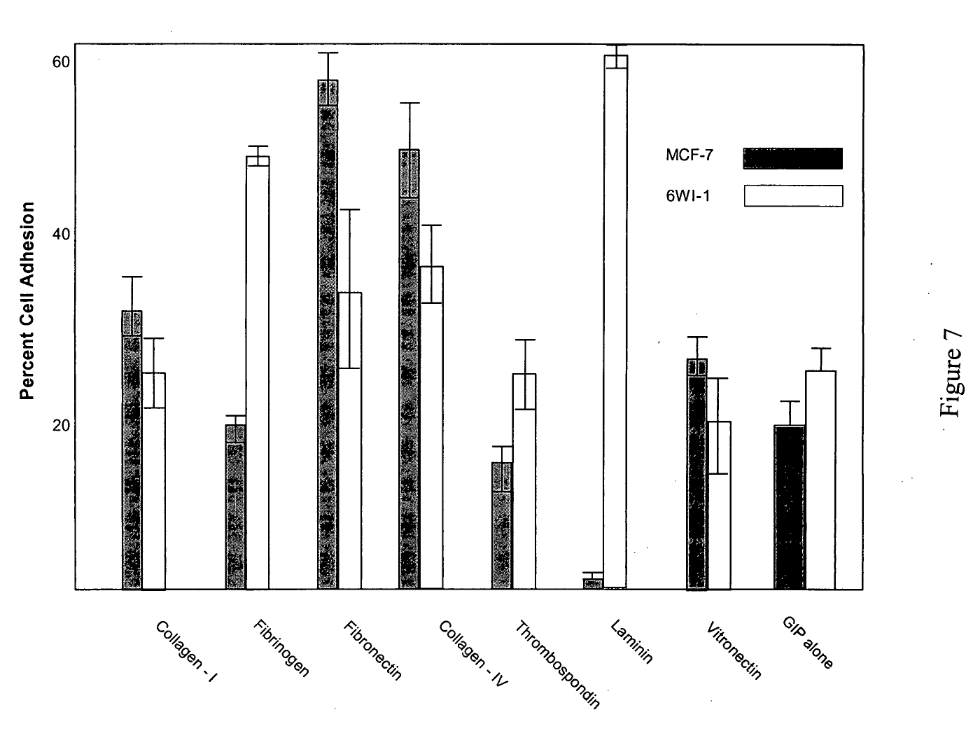

The 34-mer GIP (P149), and its fragment P149c, but not P149a or P149b, significantly suppressed the accumulation of both cells and ascites fluid volume. Thus, the anti-tumor activity of P149 peptide in the mouse mammary isograft was demonstrated by the peptide's suppression of 45-55% of tumor cell proliferation and ascites fluid accumulation as compared to the vehicle control. A scrambled peptide version of the GIP totally lacked anticancer activity in the mouse mammary isograft model. See FIG. 6 & Table 1.

As an alternative model of the 6WI-I mammary tumor in adult mice, an inocula of 1×10⁶ tumor cells injected into 15-day old mouse pups provided a 6 day rather than a 12 day assay and replicated the same tumor growth suppression by GIP. Note that the P187 aspartate-mutated peptide showed slightly less growth inhibitory potency in the mouse pup model than did the original P149 peptide.

The 6WI-1 mouse mammary tumor studies involving peptide and non-peptide treatment were further compared by histopathological analysis performed on hematoxylin/eosin stained tissue sections. Table 16.

TABLE 16
6 WI-1 Mouse Mammary Tumor Metastatic Histopathological Findings*

| Treatment Group | | Tumor + Scrambled | Tumor + P149 |
|---|---|---|---|
| N = 5 | Tumor + Saline | Peptide | Peptide |
| Gross Findings | Large tumor masses | Presence of tumor | Reduced tumor masses |
| | around intestine and | masses and foci | and foci |
| | colon | | |
| Liver | No infiltrate | No infiltrate | No infiltrate |
| Spleen | No infiltrate | No infiltrate | No infiltrate |
| Kidney | No infiltrate | No infiltrate | No infiltrate |
| Pancreas | Massive tumor infiltrate | Distinct presence of | Minimal tumor |
| | and necrosis | tumor infiltrate | infiltrates; lymphoid |
| | | | response |
| Uterus + Ovary | Tumor encased organ | Tumor encased organ | Minimal tumor presence |
| | serosa | serosa | on organ serosa |
| Retro-peritoneal | Extensive tumor infiltrate | Extensive tumor infiltrate | Some tumor infiltrate; |
| mesentery and fat | in fat tissue | and necrosis | lymphoid response |
| Abdominal Muscle | Multiple tumor infiltrate | Focal tumor infiltrate | Minimal tumor presence |
| Comment | Extensive tumor | Tumor infiltrate on | Overall diminished tumor |
| | encasement of organs | organs and in mesentery | infiltrates throughout |
| | and bowels | especially fat tissue | |

*1 × 106 tumor cell inoculation I.P. 0.1 cc treatment injections given daily for eleven days using a filtered tuberculin syringe (26 gauge needle).

It was readily apparent that the mammary tumor cells were highly invasive to many organs and tissues within the body cavity. Surprisingly, the liver, spleen, and kidney showed no apparent metastasis, invasion, or infiltration possibly due to a difference in organ encapsulation. However, the most extensive organ invasion and infiltration of tumor cells occurred in the retroperitoneal interface of the pancreas. The tumor was selective for the parietal and pelvic peritoneum, especially the retroperitoneal serosa and fatty tissues of the uterus, ovary, and kidney.

In conclusion, mammary tumor cells were identified in all 3 treatment groups with a decreasing pattern of metastatic infiltration depending on the treatment group i.e., saline>scrambled peptide>GIP. Large tumor masses, however, were observed in the saline-treated group. and high-to-moderate tumor infiltrates in the control peptide, the GIP-treated groups displayed only minimal tumor infiltrates associated with foci of lymphoid cells representing. an inflammatory response. The GIP-treated mice could be distinguished from the other groups in that the pleotrophic lymphoid-associated infiltrates were not observed in the saline and control peptide treated animals.

### Example 7

### GIP Effects on Platelet Aggregation

The ability of GIP and its fragments were individually examined for aggregation inhibition using citrated human platelet-rich plasma (PRP). The peptides were freshly dissolved in 0.15 M NaCl containing 2.5×10⁶ platelets/ml. The various platelet agonists used as activating agents were ADP at 3 μM final concentration, collagen at 2 to 5 μg/ml PRP, arachidonic acid at 300 μM and epinephrine at 5 μM. When concentrations of ADP were added to the platelet rich plasma, the two phases of aggregation fuse to give a large aggregation response that does not reverse rapidly. GIP was able to dramatically inhibit the secondary ADP-induced aggregation response. Peptide P149 at 100-300 μM PRP inhibited ADP-induced platelet aggregation by 96%; while at 10 mg peptide, inhibition did not occur. It was also shown that the P149b middle peptide also inhibited ADP-induced aggregation, with an initial slight delay followed by a 80-90% inhibition, which was not quite as dramatic as P149 alone.

Further characterization of the ADP-induced inhibition by GIP included a titratable decline in surface-expressed CD62 (P-selectin) fluorescent staining of ADP-activated platelet studied by flow cytometry. The P-selectin protein is a 140 Kd platelet surface marker induced by thrombin which is expressed on platelet endothelial cells, and megakaryocytes. Chen et al., “Geometric control of cell life and death” Science 276:1425-1428 (1997). Platelet selectin mediates platelet and neutrophil adhesion to endothelial cells during the adherent and rolling stages of neutrophil migration along the inner vascular endothelium. It is of interest that GIP also has been shown to inhibit agonist-induced thrombin and tissue factor responses in immature rats. Mizejewski et al., “Alpha-fetoprotein derived synthetic peptides: assay of an estrogen-modifying regulatory segment” Mol Cell Endocrinol 118:15-23 (1996); Murawaki et al., “Serum tissue inhibitor of metalloproteinases in patients with chronic liver disease and with hepatocellular carcinoma” Clin Chim Acta 218:47-58 (1993).

Using collagen as a second activator of platelet aggregation, P149 (100 μg) was capable of inhibiting>90% of the collagen-induced secondary aggregation response. See FIG. 10B. However, no significant inhibition was observed using P149c with both collagen and ADP-activated platelets. Both P149a and P149b were capable of inhibiting by >70% the platelet aggregations induced by ADP and collagen. Thus, two subfragments of P149 were capable of inhibiting platelet aggregation to a lesser extent than total P149, however, the aggregation inhibition was still significant.

The third platelet activation was induced by arachidonic acid (AA) whose reaction commenced immediately after addition of AA to the bath. See FIG. 10A. Moreover, addition of P149 or P149b resulted in complete aggregation inhibition for several minutes until epinephrine (which was non-GIP reactive) was added to the incubation bath. Complete platelet aggregation then occurred reaching nearly 100% indicating that, epinephrine employed different cell surface receptors sites for platelet aggregation. Further confirmation of the. P149 inhibition of AA-induced aggregation was shown by fluorescent quenching of CD62P platelet surface marker in association with AA-activated platelets.

### Example 8

### GIP Effects on Cell Cycle Regulation and Radiosensitivity

Programmed cell death, or apoptosis, is a physiological process of cellular demise in contrast to necrosis or autophagy. During embryonic/fetal development, cellular apoptosis occurs in the process of forming cavities, vessels, and ducts during histogenesis and organogenesis. For survival, cells undergo rescue from apoptosis by activation of a gene family of proteins call bc1-2. Thus, there is a delicate balance between cell death and survival, regulated by different families of proteins.

GIP's effect on cell apoptosis was assayed by in vitro flow cytometry in isolated human lymphocyte, monocyte, and thymocyte cell populations. Apoptosis can be induced either by dexamethasone administration to these cell types or by gamma irradiation. The effect of GIP was assayed in these non-tumor cells suspensions incubated overnight at 37° C. in dexamethazone or after exposure to gamma radiation in the presence of 10⁻⁸ to 10⁻¹⁰ M GIP.

Following flow cytometric analysis, it was found that GIP enhanced apoptosis in irradiated thymocytes, but not in lymphocyte or monocyte cell populations. That suggested that GIP could be a cell radiosensitizing agent for some cells. Furthemore GIP did not block dexamethasone induced cell death. See FIG. 23.

Future data will be mined to examine the possibility that GIP treatment may enhance radiosensitivity of breast tumor cells. There is a desire clinically to minimize the level of radiation therapy administered to reduce adverse treatment side effects. In theory, cells in G1 and M phases of the cell cycle should be very sensitive to radiation therapy at low dose. However, most compounds that arrest the cell cycle at these stages also increase the level of anti-apoptotic proteins, and the net effect is reduced tumor radiosensitivity. Mining of data obtained as described below should predict whether GIP has potential to enhance radiosensitivity of breast tumor cells and block the ability of these cells to escape radiation induced apoptosis.

Global gene expression in MCF-7 control cells, and in MCF-7 cells treated with the cyclic peptide for 8 days will be determined with microarray analysis. After treating the cell cultures for 8 days and collecting high quality RNA, the microarray services provided by Yale Genomic Core will be used to examine changes in levels of over 40,000 transcripts spotted on the Affymetrix human 2.0 Plus gene chip. Biological networking software, NetAffx, and Ingenuity Pathways Analysis will be used to examine changes in gene expression of pathways controlling cell cycle. Changes in gene expression detected by microarray analysis will be confirmed with Q-ERT-PCR. Specifically changes for any of the eight genes (supra) and already designed primers (See Table 11) and established standard curves will be confirmed. An additional five genes will be selected based on results from the microarray analysis will also have their expression level confirmed. For these five new genes, primers will be designed and tested, standard curves established and expression in breast cancer cells treated with the cyclic peptide confimed. To determine whether protein levels of selected genes have changed due to treatment with the cyclic form of GIP, Western Blot will be conducted using antibodies to p14, p19, p21, p27, Rb1, and, Cyclin E, as well as the phosphorylated form of p27.

Analysis of this data will provide information regarding the effect of GIP on the various families of proteins regulating the delicate balance between cell apoptosis and cell survival.

### Example 9

### GIP Effects on Nonselective Calcium Channel Regulation

LNCaP human prostate tumor cells were cultured on 5×5 mm plastic coverslips and were transferred to a microperfusion chamber on the stage of an inverted microscope. Whole-cell voltage clamp was accomplished with an Axopatch 200B (Axon Instr. Co., Union City, Calif.) using ruptured patches. Borosilicate glass capillaries (1.2 mm OD, 0.68 ID, type EN-I, Garner Glass Co., Claremont, Calif.) were used to fabricate patch pipettes (3-8 MΩ in the bath solution) with a Brown-Flaming horizontal micropipette puller (P-87, Sutter Instruments, San Rafael, Calif.). Patch-pipette tips were heat-polished prior to use. A micromanipulator (Narishige) fixed to the microscope positioned the pipettes. Electrode capacitance and access resistance were compensated prior to measurements. All experiments will be performed at room temperature (20-22° C.). The standard extracellular salt solution for ion currents contained (in mM): 150 NaCl, 6 KCl, 1.5 CaCl₂, 1 MgCl₂, 10 HEPES, 10 glucose, and pH adjusted to 7.4 with 1 N NaOH. The internal (pipette) solution for measuring ion currents contained (in mM): 140 K gluconate, 2 MgCl₂, 1 CaCl₂, 10 EGTA, 10 HEPES, pH 7.2 with KOH. In some experiments, external solution was exchanged for one in which cations were substituted with an impermeable organic cation (in mM): 156 N-methyl-d-glucamine (NMDG), 1 MgCl₂, 10 HEPES, 10 glucose, pH 7.4 with 1 N HCl. Electrophysiological recordings were procured and analyzed using PClamp8 software (Axon Instr. Co, Union City, Calif.) and the WinASCD software (Guy Droogmans, Katholieke Universiteit; Leuven, B E). All results were recorded on computer hard drive or on digital audio tape (DAT).

FIG. 19 illustrates the basic recording configuration. A glass micropipette is positioned against the cell's plasma membrane. Slight suction is applied and the membrane seals to the glass. Gigohm resistance of this juxtaposition is necessary for accurate membrane current measurements. Once the seal is formed, further suction is applied to rupture the membrane patch. Now the contents of the pipette dialyze the cell and one can now clamp the transmembrane potential (i.e., for example, a voltage clamp) and measure the corresponding membrane current by virtue of the “feedback resistors” and operational amplifiers in the equipment. Then, either by “voltage ramps” or “voltage steps” one can obtain plots of membrane current vs. voltage. The slope conductance, G, which is the inverse of membrane resistance, and the reversal potential, Vr, are both important variables. Changes in the first variable reflect changes in the number of ion channels functioning at a given moment, Increases in the time channels are open or increases in the number of channels in the membrane decrease the membrane resistance, which results in an increase in membrane conductance. Changes in the second variable indicate which ions are moving through the channels, depending on the spectrum of ions in the pipette and in the bathing solution.

Results:

Whole cell membrane currents from LNCaP cells are shown in FIG. 20. Under control conditions, in which the cell is bathed with standard external salt solution, a substantial inward current was evident at negative membrane potentials (black trace). A corresponding outward current was also evident at membrane potential greater than 50 mV. These findings are consistent with previous reports of voltage-gated K⁺ channels and non-selective cation currents. Lanaido et al., The Prostate 46:262 (2001); and Sydorenko et al., J. Physiol. 548:823 (2003), Vanden Abeele et al., Cell Calcium 33:357 (2003), respectively. Substitution of the standard external solution with one containing NMDG eliminated the inward current. See FIG. 20 (red trace). The inward current was restored on washout of NMDG with standard external solution. See FIG. 20 (green trace).

Addition of linear GIP (10 μM) increased both inward and outward whole-cell current in LNCaP cells. See FIG. 21. These increases occurred within minutes, and the increase in outward current was greater than that of the inward current. See FIG. 21A. Substitution of permeable cations with NMDG resulted in diminution of both the inward and outward current. FIG. 21A. Individual traces of the I-V curves taken at times indicated by the corresponding numbers and reverse fill symbols are shown. See FIG. 21B. Changes in slope conductance by either added GIP or substituted NMDG resulted without changes in corresponding reversal potentials, which were near zero.

Further effects on GIP (linear, cyclic, and scrambled) on LNCaP cells were evaluated by comparing their effects on slope conductance. FIG. 22. Addition of scrambled GIP sequences had no effect on membrane current of LNCAP cells. FIG. 22A. However, substantial increases in slope conductance (i.e., for example, a decrease in membrane resistance) resulted from linear GIP and to a lesser degree by added cyclic GIP. FIGS. 22B & 22C, respectively. Average values of repeated measures of slope conductance under control conditions and each of those after addition of scrambled, linear or cyclic GIP are shown. See Table 17.

TABLE 17
Membrane Conduction Effects Induced By GIP-derived Peptides

| | Scrambled GIP | | Linear GIP | | Cyclic GIP |
|---|---|---|---|---|---|
| Control | (10 μM) | Control | (10 μM) | Control | (10 μM) |
| 0.63 0.14 | 0.57 0.12 | 0.53 0.20 | 0.84 0.26* | 0.65 0.14 | 0.78 0.15* |
| nS/pF, n = 4 | nS/pF, n = 4 | nS/pF, n = 5 | nS/pF, n = 5 | nS/pF, n = 5 | nS/pF, n = 5 |

*Differs from paired control, p < 0.05.
LNCaP Cells. Voltage Ramp: −100-100 mV (400 ms duration).

These data show that both linear and cyclic GIP increased slope conductance of LNCaP cells, but the scrambled sequence was without effect.

### Example 10

### GIP Effects on Cancer Cell Culture Spreading

The P149 treated, untreated and ovalalbumin peptide-coated coverslips were placed in individual 24-well microtiter plates for MCF-7 cell plating. After the microtiter plates were incubated for 22-24 hours, the cells were fixed in 0.5 mL of 37% formaldehyde-stained with crystal violet, and counted under light microscopy. Results of the cell spreading/migration studies revealed that the GIP peptide-coated surfaces inhibited cell spreading throughout an increasing concentration range nom 0.5 μg to approximately 10 μg/ml. See FIG. 11. The cells, unable to migrate, displayed distorted morphology such as star-shaped configurations, cytoplasmic spiking, surface spiny spheres, extended cytoplasmic processes, and low cell viability. These data support the cell adhesion studies described above and re-affirm that GIP, and its peptide fragments, are associated with cell surface activities and cell-to-cell interactions related to integrin activities with the ECM such as basement membranes, interstitial surfaces, and connective tissues.

### Example 10

### GIP Effects on Cell Cycle mRNA Expression

Materials and Methods

All peptides were custom synthesized by Peptides International, Louisville, Ky. Peptide quality control procedures included reserve-phase HPLC, mass spectroscopy, and gel filtration HCPL as previously reported Eisele et al., “Studies on analogs of a peptide derived from alpha-fetoprotein having antigrowth properties” J. Peptide Res. 57:539-546 (2001); MacColl et al., “Interrelationships among biological activity, disulfide bonds, secondary structure, and metal ion binding for a chemically synthesized 34-amino-acid peptide derived from alpha-fetoprotein” Biochem. Biophys. Acta. 1528:127-134 (2001); Butterstein et al.,. “Biophysical studies and anti-growth activities of a peptide, a certain analog and a fragment peptide derived from alpha-fetoprotein” J. Peptide Res 61:213-218 (2003). A total RNA isolation kit (Ambion Inc.), and associated chemicals were obtained as Tri Reagent from Molecular Research Center, Inc. The RNA was quantitated using Genequant Pro from Amersham Biosciences, Inc. The one-step RT-PCR kit was purchased from Qiagen, Inc and the SYBR Green fluorescent nucleic acid stain was obtained from Molecular Probes Corp. The Taqman primer and probe sequences were constructed by methods known in the art.

Cell Culture:

MCF-7 cell cultures were maintained in DC5 medium consisting of Dulbecco's Modified Eagle Medium (without phenol red) supplemented with 5% cosmic calf serum, insulin, and other components as previously described. Gierthy et al., “Estrogen-stimulation of postconfluent cell accumulation and foci formation of human MCF-7 breast cancer cells” J. Cell. Biochem. 45: 177-187 (1991). Cells were passaged at ˜80% confluence and maintained in a 37° C. humidified incubator with 5% CO₂.

MCF-7 Cell Proliferation Assay:

MCF-7 cells were seeded at 25,000 cells/well in 48 well microtiter plates, and incubated for eight days in the various test and control reagents. The cells were incubated in culture medium containing charcoal/dextran-treated (stripped) bovine calf serum (BCS) and the test compounds, including linear cyclic GIP, and amino acid-scrambled GIP at concentration ranging from 10⁻⁸ to 10⁻¹² Molar. Tumor cells were fixed after 8 days and analyzed for proliferation using the standard sulfarhodamine B staining procedure.

Quantitative RT-PCR

Cell culture, treatment, and RNA isolation:

MCF-7 cells were seeded at 1×10⁶ cells per well in 6-well plates. The cells were incubated for 192 h in medium containing charcoal/dextran-treated BCS and test compounds described above in the proliferation assay. Peptide was administered each day for 8 days. Total RNA was isolated using the RNAqueous 4 PCR kit (Ambion Inc.) or Tri Reagent (Molecular Research Center, Inc.). The quality of the RNAs were assessed by electrophoresis on denaturing agarose gels and were quantified at 260 nm with the Genequant Pro (Amersham Biosciences).

Quantitative Reverse-Transcriptase Polymerase Chain Reaction

Details for the quantitative RT-PCR protocol were previously reported. Fasco et al., “Quantitative RNA-polymerase chain reaction-DNA analysis by capillary electrophoresis and laser-induced fluorescence” Anal. Biochem. 224:140-147 (1995). Briefly, RNA samples were reverse transcribed and amplified using the One Step RT-PCR kit (Qiagen Inc.) in the Roche Light Cycler. Total RNA was diluted to 0.1 mg/ul for all genes assayed except for 28S, which was diluted to 0.001 mg/ml. Diluted total RNA (0.75 ml) was incubated with Qiagen RT-PCR master mix and was reverse transcribed for 30 min at 50° C. Following reverse transcription, samples were heated for 15 min at 95° C. to activate the HotStarTaq DNA polymerase and to simultaneously inactivate the reverse transcriptase. Gozgit et al., “Differential action of polycyclic aromatic hydrocarbons on endogenous estrogen-responsive genes and on a transfected estrogen-responsive reporter in MCF-7 cells” Toxicol. & Appl. Pharmacol. 196:58-67 (2004).

Gene-specific primers (25 m) were used for the following: Cyclin D3, D1, CDK2, CDK4, CDK6, RB1, Ki67, Cyclin E1, Cyclin BI, and with B-Actin serving as housekeeping (control) gene. The generation of amplified products was monitored over 45 PCR cycles by fluorescence of intercalating SYBR Green I nucleic acid gel stain (Molecular Probes). PCR products were examined with melting curve analysis (Roche Light Cycler) and run on a standard 1.3% TAE agarose gel to ensure validity and purity. Results are expressed as relative mRNA levels (arbitrary units) calculated from a standard curve for each gene using the Roche Light Cycler software. The standard curves were generated from total RNA isolated from MCF-7 cells treated as described above. Following a 1:10 serial dilution, the RNA was run in triplicate in the Roche Light Cycler, where it was stored as a standard curve and used for quantification of relative mRNA levels for each gene (Gozgit et al, supra).

Western Blot Analysis of Cell Cycle Kinase Inhibitors

Cultured cells (wet weight 10-20 mg) were homogenized in WB-HB buffer (10 mM Tris-HCl (pH 7.4), 150 mM NaCl, 0.5% Triton X-100 and 0.2 mM phenylmethyl sulfonyl fluoride) with a Polytron homogenizer (Kinematics, Luzern, Switzerland). The protein concentration of samples was measured. Bradford, M. A, “Rapid and sensitive method for the quantitation of microgram quantities of protein utilizing the principle of protein-dye binding” Anal. Biochem. 72:315-323 (1976). Each sample (25 ml) containing 100 mg of protein was added to 25 ml of a sample buffer (12.5 mM Tris-HCl (pH 618), 2% glycerol, 0.4% SDS and 1.25% 2-mercapto-ethanol) and analyzed by 7.5% SDS-PAGE under non-reducing conditions. The gel was transferred to a nitrocellulose membrane (Hybond ECL Western; Amersham, Arlington Heights, Ill.). The membrane was blocked with 5% milk (from dehydrate) in a blocking buffer (20 mM Tris-HCl (pH 7.6), 137 mM NaCl and 0.1% Tween-20), incubated with rabbit anti-P27 and/or P21 antibody (1:1000) (Oncogene Research Products, Cambridge, Mass.), washed and then incubated with peroxidase-linked species-specific whole antibody, anti-rabbit immunoglobulin from a donkey (1:2000) Amersham, Buckinghamshire, UK). Specific bands were detected with ECL chemiluminescence reagent (Amersham) and X-ray film was exposed on the membrane at room temperature for 10 min.

Statistics

The levels of cell cycle proteins and their mRNA were measured from 2 replicates from the tumor lysates; the assay for each sample was carried out in triplicate. Statistical analysis was performed with Student's t-test between the housekeeping gene, Actin and the cell cycle mRNA constituents using the one-way ANOVA among actin mRNA cell cycle mRNA constituent and in the proliferation assays; statistical differences were considered significant when the P-value was less than 0.05.

Results:

Inhibition of Cell Proliferation:

MCF-7 cells incubated in charcoal-stripped calf serum were growth suppressed at culture day-8 using the cyclic form of GIP at 10⁻⁵ to 10⁻¹⁴ M concentrations. The data indicated that GIP was most effective at 10⁻⁸ M but was dose dependent from 10⁻⁸ to 10⁻¹³ M. These are physiological doses and the effect appeared to be cytostatic and not cytotoxic to the MCF-7 cells. Cell viability (>95%) was performed by trypan blue determination. The growth arrest was highly significant by statistical ANOVA analysis (P-Value=1.8 E-10).

Gene Expression Analysis

Gene expression was determined by quantitative RT-PCR using gene-specific primers and measuring fluorescence of intercalating SYBR Green I. Quantitative RT-PCR products were visualized by ethidium bromide staining on a 1.3% agarose gel confirming size-specific, single bands. No significant differences in total RNA levels were observed among samples by estimate of the 28S ribosomal RNA subunit mRNA content. As anticipated, GIP showed both dose dependent down-regulation or up-regulation in the various cell cycle-associated mRNA constituents.

For example, GIP induced a significant down regulation of Cyclin-E1 at a peptide dose range of 10⁻⁵ to 10⁻¹⁴ M representing fold decreases of 2.1 to 4.6. It was noteworthy that mRNA Cyclin-E expression was reduced even up to 10⁻¹³ M concentration. In contrast, GIP treatment produced a 1.7 fold increase in mRNA cyclin D1 in conjunction with a cdk4 induction of 2.4 fold increase, and an Rb1 induction ranging form 2.4 to 3.4 fold increases. In comparison, GIP treatment of MCF-7 cells had no effect on Ki67, cdk2, cdk6, and Cyclin B1 mRNA levels. In summary, treatment of MCR-7 breast cancer cells by GIP resulted in down-regulation of only Cyclin E1, but up-regulations of Cyclin D1, cdk4, and Rb1 (10-10M). No effect was observed on Ki67, cdk2, cdk6, and Cyclin B1.

Western Blot Analysis: CdK Inhibitors

The analysis of protein content by Western Blot analysis was determined for the cdk inhibitors p21 (INK) and p27 (Kip/Cip). The presence of banding observed on the nitrocellulose transfer gels clearly indicated a normal presence of p21 and p27 in the MCF-7 lysates as compared to the β-actin control. In contrast, the presence of phosphorylated p27 was strikingly depleted. Since the lysates were prepared from cell cultures treated at 3 GIP concentrations (10⁻⁶ 10⁻⁹, 10⁻¹² M), it was evident that extremely low concentrations of GIP (10-12 M) were effective at preventing p21 phosporylation. In summary, it was demonstrated that GIP had no effect on non-phosphorylated p27; rather, GIP inhibited the formation of the phosphorylated form of p27 in MCF-7 cell lysates and lacked a dose response extending from 10⁻⁶ to 10⁻¹² M concentrations.

We claim:

1. A growth inhibitory protein-derived fragment comprising homology to SEQ ID NO:2.

2. The fragment of claim 1, wherein said fragment comprises a matrix metalloproteinase-associated peptide comprising at least a portion of LSX₁DX₂X₃X₄ACGEGX₅AX₆₁X₇X₈GHX₉X₁₀X₁₁RHX₁₂X₁₃X₁₄PX₁₅X₁₆PGVG (SEQ ID NO:75).

3. The fragment of claim 1, wherein said fragment comprises an extracellular matrix-associated peptide comprising at least a portion of LSEX₁KLLX₂CGX₃GX₄X₅X₆LX₇X₈X₉HLX₁₀IX₁₁HX₁₂X₁₃X₁₄PX₁₅X₁₆PGVG (SEQ ID NO:74).

3. The fragment of claim 1, wherein said fragment comprises a clotting associated peptide comprising at least a portion of X₁X₂LX₃CX₄X₅GX₆X₇X₈₋X₉X₁₀X₁₁GHLCIRX₁₂X₁₃X₁₄₋X₁₅PX₁₆NPX₁₇X₁₈G (SEQ ID NO;76)

4. The fragment of claim 1, wherein said fragment comprises a cation channel peptide comprising at least a portion of LSEDKLLACGEGX₁QDIIIGHX₂CIRHEMTPVNPGVG (SEQ ID NO:77)

5. The fragment of claim 1, wherein said fragment comprises a antiangiogenic peptide comprising at least a portion of LSEDKLLX₁CGEX₂X₃ADIX₄IX₅H-X₆CIRHEMTPVNPX₇X₈X₉ (SEQ ID NO:81).

6. The fragment of claim 1, wherein said fragment comprises a cytoskeletal regulator peptide comprising at least a portion of LSEDKLLX₁CGEGX₂ADIIIGHX₃CIRHEMTPVNPGV (SEQ ID NO:82).

7. The fragment of claim 1, wherein said fragment comprises a cell cycle regulator peptide comprising at least a portion of X₁LLX₂CGEGAADIIIGHX₃CIRX₄EX₅TPVNPX₆X₇ (SEQ ID NO:83).

8. The fragment of claim 1, wherein said fragment comprises a metabolic enzyme regulator peptide comprising at least a subfragment of LSEDKLLACGEX₁X₂ADIIIGHX₃CIRHEMTPVNPGVG (SEQ ID NO: 84).

9. The fragment of claim 1, wherein said fragment comprises a transcription regulator peptide comprising at least a subfragment of LSEDKLLX₁CGEGAADIIIGHLCIRHEMTPVNPX₁X₂ (SEQ ID NO: 85).

10. A method comprising ii) identifying disease specific regulatory proteins; ii) matching SEQ ID NO:2 to said regulatory proteins amino acid sequence; and iii) maximizing identity and similarity values, such that a growth inhibitor protein-derived peptide fragment may be synthesized based upon said regulatory protein homology to said SEQ ID NO:2.

11. The method of claim 10, wherein said matching is selected from the group consisting of sequence reversal, D-amino acid replacement, coded amino acid pairing, non-coded amino acid pairing, and cargo bay motifs.

12. The method of claim 11, wherein said cargo bay motifs comprise, nuclear transcription, decoy growth factor, nuclear localization signals, transforming growth factor, apoptosis FAS, apoptosis inhibition, fibroblast growth factor receptor-actor agonists, viral cloaking, chemokine decoy ligands, chemokine CSCR4 decoy receptors, and soluble D6 chemokine receptors.


SEQUENCE LISTING (181 sequences)

SEQ ID NO: 1 - protein, 590 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - protein, 34 aa, Homo sapiens
LSEDKLLACGEGAADIIIGHLCIRHEMTPVNPGV

SEQ ID NO: 3 - protein, 12 aa, Homo sapiens
LSEDKLLACGEG

SEQ ID NO: 4 - protein, 14 aa, Homo sapiens
AADIIIGHLCIRHE

SEQ ID NO: 5 - protein, 8 aa, Homo sapiens
MTPVNPGV

SEQ ID NO: 6 - protein, 10 aa, Homo sapiens
HEMTPVNPGV

SEQ ID NO: 7 - protein, 19 aa, Homo sapiens
LLACGEGAADIIIGHLCIR

SEQ ID NO: 8 - protein, 5 aa, Homo sapiens
LSEDK

SEQ ID NO: 9 - protein, 34 aa, Homo sapiens
  misc_feature (9)..(9): Xaa can be any naturally occurring amino acid
LSEDKLLAXGEGAADIIIGHLXIRHEMTPVNPGV

SEQ ID NO: 10 - protein, 12 aa, Homo sapiens
  misc_feature (9)..(9): Xaa can be any naturally occurring amino acid
LSEDKLLAXGEG

SEQ ID NO: 11 - protein, 19 aa, Homo sapiens
  misc_feature (4)..(4): Xaa can be any naturally occurring amino acid
LLAXGEGAADIIIGHLXIR

SEQ ID NO: 12 - protein, 14 aa, Homo sapiens
  misc_feature (10)..(10): Xaa can be any naturally occurring amino acid
AADIIIGHLXIRHE

SEQ ID NO: 13 - protein, 40 aa, Homo sapiens
KDFIHKQGVALQTMKQELINLVKELVKPQIEAVADFSGLL

SEQ ID NO: 14 - protein, 34 aa, Homo sapiens
HPEAKRMPCAEDYLSVVLNQLCVLEHKTPVSDRV

SEQ ID NO: 15 - protein, 34 aa, Homo sapiens
GINHLPSDAVGTAIEVMPCLRDGKLCIGAEHIEL

SEQ ID NO: 16 - protein, 13 aa, Homo sapiens
LSEDKLLACGEGD

SEQ ID NO: 17 - protein, 16 aa, Homo sapiens
EGAADIIIGHLCIRHE

SEQ ID NO: 18 - protein, 11 aa, Homo sapiens
EMTPVNPGVGN

SEQ ID NO: 19 - protein, 16 aa, Homo sapiens
DCIRHEMTPVNPGVGD

SEQ ID NO: 20 - protein, 11 aa, Homo sapiens
CQLSEDKLLAC

SEQ ID NO: 21 - protein, 14 aa, Homo sapiens
CGEGAADIIIGHLC

SEQ ID NO: 22 - protein, 16 aa, Homo sapiens
CIRHEMTPVNPGVGQC

SEQ ID NO: 23 - protein, 8 aa, Homo sapiens
LSEDKLLE

SEQ ID NO: 24 - protein, 8 aa, Homo sapiens
ECGEGAAD

SEQ ID NO: 25 - protein, 10 aa, Homo sapiens
DIIIGHLCIR

SEQ ID NO: 26 - protein, 22 aa, Homo sapiens
LSEDKLLACCIRHEMTPVNPGV

SEQ ID NO: 27 - protein, 34 aa, Homo sapiens; identical to SEQ ID NO: 2

SEQ ID NO: 28 - protein, 11 aa, Homo sapiens
EAADIIIGHLD

SEQ ID NO: 29 - protein, 19 aa, Homo sapiens
LSEDKLLACGEGAADIIIE

SEQ ID NO: 30 - protein, 10 aa, Homo sapiens
DIGHLCIRHE

SEQ ID NO: 31 - protein, 9 aa, Homo sapiens
TPVNPGVGD

SEQ ID NO: 32 - protein, 9 aa, Homo sapiens
KLLACGEGE

SEQ ID NO: 33 - protein, 21 aa, Homo sapiens
DLLACGEGAADIIIGHLCIRK

SEQ ID NO: 34 - protein, 21 aa, Homo sapiens
DLLACGEGAADIIIGHLCIRK

SEQ ID NO: 35 - protein, 28 aa, Homo sapiens
ECGEGAADIIIGHLCIRHEMTPVNPGVD

SEQ ID NO: 36 - protein, 28 aa, Homo sapiens
ECGEGAADIIIGHLCIRHEMTPVNPGVD

SEQ ID NO: 37 - protein, 14 aa, Homo sapiens
ECIRHEMTPVNPGD

SEQ ID NO: 38 - protein, 8 aa, Homo sapiens
ETPVNPGD

SEQ ID NO: 39 - protein, 13 aa, Homo sapiens
DAADIIIGHLCIR

SEQ ID NO: 40 - protein, 10 aa, Homo sapiens
HEMTPVNPGE

SEQ ID NO: 41 - protein, 34 aa, Homo sapiens
VGPNVPTMEHRICLHGIIIDAAGEGCALLKDESL

SEQ ID NO: 42 - protein, 34 aa, Homo sapiens; identical to SEQ ID NO: 2
  MOD_RES (1)..(34): These residues are D-amino acids.

SEQ ID NO: 43 - protein, 34 aa, Homo sapiens
DSLLFNERTPLPRRLYYYPINTYSILYWGHLGPQ

SEQ ID NO: 44 - protein, 34 aa, Homo sapiens
EWDNYEDRTPLPRRLFWHPINTHTILWSGWLGPQ

SEQ ID NO: 45 - protein, 11 aa, Homo sapiens
YGRKKRRQRRR

SEQ ID NO: 46 - protein, 34 aa, Homo sapiens
LYGRKKRRQRRRAADIIIGHLCIRHEMTPVNPGV

SEQ ID NO: 47 - protein, 12 aa, Homo sapiens
MYIEALDIIKYA

SEQ ID NO: 48 - protein, 34 aa, Homo sapiens
LSEDKLLACMYIEALDIIKYACIRHEMTPVNPGV

SEQ ID NO: 49 - protein, 4 aa, Homo sapiens
RRKK

SEQ ID NO: 50 - protein, 5 aa, Homo sapiens
LVQLL

SEQ ID NO: 51 - protein, 3 aa, Homo sapiens
TTA

SEQ ID NO: 52 - protein, 34 aa, Homo sapiens
LSERRKKRCGEGLVQLLTTALCIRHEMTPVNPGV

SEQ ID NO: 53 - protein, 21 aa, Homo sapiens
VSLTKLLAAGEGCVVGYIGER

SEQ ID NO: 54 - protein, 34 aa, Homo sapiens
VSLTKLLAAGEGCVVGYIGERCIRHEMTPVNPGV

SEQ ID NO: 55 - protein, 9 aa, Homo sapiens
VPIAQKSEP

SEQ ID NO: 56 - protein, 34 aa, Homo sapiens
LSEDKLLACGEGAVPIAQKSEPIRHEMTPVNPGV

SEQ ID NO: 57 - protein, 10 aa, Homo sapiens
RQGYRVFSLG

SEQ ID NO: 58 - protein, 2 aa, Homo sapiens
SL

SEQ ID NO: 59 - protein, 34 aa, Homo sapiens
LSEDKLLACGEGRQGYRVFSLGIRHEMTPVNSLV

SEQ ID NO: 60 - protein, 8 aa, Homo sapiens
RRLKKAAV

SEQ ID NO: 61 - protein, 10 aa, Homo sapiens
ALLPAVLLAP

SEQ ID NO: 62 - protein, 34 aa, Homo sapiens
LSRRLKKAAVALLPAVLLAPLCIRHEMTPVNPGV

SEQ ID NO: 63 - protein, 1 aa, Homo sapiens
I

SEQ ID NO: 64 - protein, 9 aa, Homo sapiens
SEDKKLWAS

SEQ ID NO: 65 - protein, 8 aa, Homo sapiens
DILRNIAR

SEQ ID NO: 66 - protein, 10 aa, Homo sapiens
AQVGDSVGVA

SEQ ID NO: 67 - protein, 34 aa, Homo sapiens
ISEDKKLWASEDILRNIARHLAQVGDSVGVAPGV

SEQ ID NO: 68 - protein, 13 aa, Homo sapiens
ASDLAIDLYHIRT

SEQ ID NO: 69 - protein, 34 aa, Homo sapiens
LSEDKLLACGEGASDLAIDLYHIRTEMTPVNPGV

SEQ ID NO: 70 - protein, 12 aa, Homo sapiens
ISLDRYLAIVHA

SEQ ID NO: 71 - protein, 34 aa, Homo sapiens
LSEDKLLACGEGISLDRYLAIVHATEMTPVNPGV

SEQ ID NO: 72 - protein, 13 aa, Homo sapiens
ISLDRYLEIVHAT

SEQ ID NO: 73 - protein, 34 aa, Homo sapiens
LSEDKLLACGEGISLDRYLEIVHATEMTPVNPGV

SEQ ID NO: 74 - protein, 35 aa, Homo sapiens
  MISC_FEATURE (4)..(4): X is Asp, Ile, Gln, Arg, or Thr.
LSEXKLLXCGXGXXXIXXXHLXIXHXXXPXXPGVG

SEQ ID NO: 75 - protein, 35 aa, Homo sapiens
  MISC_FEATURE (3)..(3): X is Glu or Asp.
LSXDXXXACGEGXAXIXXGHXXXRHXXXPXXPGVG

SEQ ID NO: 76 - protein, 31 aa, Homo sapiens
  MISC_FEATURE (1)..(1): X is Lys or Gly.
XXLXCXXGXXXXXXGHLCIRXXXXPXNPXXG

SEQ ID NO: 77 - protein, 35 aa, Homo sapiens
  MISC_FEATURE (13)..(13): X is Ala or Asp.
LSEDKLLACGEGXQDIIIGHXCIRHEMTPVNPGVG

SEQ ID NO: 78 - protein, 6 aa, Homo sapiens
EWKFAR

SEQ ID NO: 79 - protein, 7 aa, Homo sapiens
GHLCIRH

SEQ ID NO: 80 - protein, 8 aa, Homo sapiens
EMTPVNPG

SEQ ID NO: 81 - protein, 35 aa, Homo sapiens
  MISC_FEATURE (8)..(8): X is Ala or Glu.
LSEDKLLXCGEXXADIXIXHXCIRHEMTPVNPXXX

SEQ ID NO: 82 - protein, 34 aa, Homo sapiens
  MISC_FEATURE (8)..(8): X is Ala or Glu.
LSEDKLLXCGEGXADIIIGHXCIRHEMTPVNPGV

SEQ ID NO: 83 - protein, 30 aa, Homo sapiens
  MISC_FEATURE (1)..(1): X is Lys or Asp.
XLLXCGEGAADIIIGHXCIRXEXTPVNPXX

SEQ ID NO: 84 - protein, 35 aa, Homo sapiens
  MISC_FEATURE (12)..(12): X is Gly or Asp.
LSEDKLLACGEXXADIIIGHXCIRHEMTPVNPGVG

SEQ ID NO: 85 - protein, 34 aa, Homo sapiens
  MISC_FEATURE (8)..(8): X is Ala or Glu.
LSEDKLLXCGEGAADIIIGHLCIRHEMTPVNPXX

SEQ ID NO: 86 - protein, 15 aa, Homo sapiens
LSEDKLLACGEGAAD

SEQ ID NO: 87 - protein, 9 aa, Homo sapiens
SEDKLLACG

SEQ ID NO: 88 - protein, 10 aa, Homo sapiens
LACGEGAADI

SEQ ID NO: 89 - protein, 7 aa, Homo sapiens
SEDKLLA

SEQ ID NO: 90 - protein, 8 aa, Homo sapiens
GEGAADII

SEQ ID NO: 91 - protein, 11 aa, Homo sapiens
SEDKLLACGEG

SEQ ID NO: 92 - protein, 12 aa, Homo sapiens
CGEGAADIIIGH

SEQ ID NO: 93 - protein, 11 aa, Homo sapiens
CGEGAADIIIG

SEQ ID NO: 94 - protein, 16 aa, Homo sapiens
GEGAADIIMTPVNPGV

SEQ ID NO: 95 - protein, 18 aa, Homo sapiens
DKLLACGEGAADIICGEG

SEQ ID NO: 96 - protein, 11 aa, Homo sapiens
  misc_feature (7)..(7): Xaa can be any naturally occurring amino acid
IRHEMTXVNPG

SEQ ID NO: 97 - protein, 28 aa, Homo sapiens
  misc_feature (21)..(21): Xaa can be any naturally occurring amino acid
CGEGAADIIIGHLCIRHEMTXVNPGVGQ

SEQ ID NO: 98 - protein, 12 aa, Homo sapiens
IRHEMTPVPPGV

SEQ ID NO: 99 - protein, 18 aa, Homo sapiens
IIGHLCIRHEMTPVNPGV

SEQ ID NO: 100 - protein, 10 aa, Homo sapiens
GEGAADIIIG

SEQ ID NO: 101 - protein, 14 aa, Homo sapiens
HLCIRHEMTPVNPG

SEQ ID NO: 102 - protein, 8 aa, Homo sapiens
DIIIGHLC

SEQ ID NO: 103 - protein, 9 aa, Homo sapiens
GEGAADIII

SEQ ID NO: 104 - protein, 7 aa, Homo sapiens
DIIGHLC

SEQ ID NO: 105 - protein, 14 aa, Homo sapiens
LSEDKSSACGEGAA

SEQ ID NO: 106 - protein, 8 aa, Homo sapiens
GHLCIRHE

SEQ ID NO: 107 - protein, 7 aa, Homo sapiens
EDKLLAC

SEQ ID NO: 108 - protein, 7 aa, Homo sapiens
GHLCIRH

SEQ ID NO: 109 - DNA, 20 bp, Artificial Sequence
  Synthetic
gtggccactaagcagaggag

SEQ ID NO: 110 - DNA, 21 bp, Artificial Sequence
  Synthetic
agcttgactagccaccgaaaa

SEQ ID NO: 111 - DNA, 20 bp, Artificial Sequence
  Synthetic
gaggaagaggaggaggagga

SEQ ID NO: 112 - DNA, 20 bp, Artificial Sequence
  Synthetic
gagatggaagggggaaagag

SEQ ID NO: 113 - DNA, 20 bp, Artificial Sequence
  Synthetic
cattcctcttcccctcatca

SEQ ID NO: 114 - DNA, 20 bp, Artificial Sequence
  Synthetic
cagggactccaaaagctctg

SEQ ID NO: 115 - DNA, 20 bp, Artificial Sequence
  Synthetic
gaaactctgaagccgaccag

SEQ ID NO: 116 - DNA, 20 bp, Artificial Sequence
  Synthetic
aggcagagattcgcttgtgt

SEQ ID NO: 117 - DNA, 20 bp, Artificial Sequence
  Synthetic
agagacaggagtggccttga

SEQ ID NO: 118 - DNA, 20 bp, Artificial Sequence
  Synthetic
tgaaagcaagcaaacaggtg

SEQ ID NO: 119 - DNA, 20 bp, Artificial Sequence
  Synthetic
ggaagcaaccctcctaaacc

SEQ ID NO: 120 - DNA, 20 bp, Artificial Sequence
  Synthetic
tttctgcttttgcattcgtg

SEQ ID NO: 121 - DNA, 20 bp, Artificial Sequence
  Synthetic
agtcagacccagtggacacc

SEQ ID NO: 122 - DNA, 20 bp, Artificial Sequence
  Synthetic
tgctgccggttaagttctct

SEQ ID NO: 123 - DNA, 20 bp, Artificial Sequence
  Synthetic
cagattgcagacctgttgga

SEQ ID NO: 124 - DNA, 20 bp, Artificial Sequence
  Synthetic
tccccgtctcccttataacc

SEQ ID NO: 125 - DNA, 20 bp, Artificial Sequence
  Synthetic
cgggaagtcactggaaacat

SEQ ID NO: 126 - DNA, 20 bp, Artificial Sequence
  Synthetic
aaacatggcagtgacaccaa

SEQ ID NO: 127 - DNA, 20 bp, Artificial Sequence
  Synthetic
ggacttcgagcaagagatgg

SEQ ID NO: 128 - DNA, 20 bp, Artificial Sequence
  Synthetic
agcactgtgttggcgtacag

SEQ ID NO: 129 - protein, 36 aa, Homo sapiens
  MISC_FEATURE (9)..(9): Methylated Cysteine.
LSEDKLLACGEGAADIIIGHLCIRHEMTPVNPGVGQ

SEQ ID NO: 130 - protein, 36 aa, Homo sapiens; identical to SEQ ID NO: 129
  DISULFID (9)..(22)

SEQ ID NO: 131 - protein, 37 aa, Homo sapiens
  MISC_FEATURE (10)..(10): Methylated Cysteine.
QLSEDKLLACGEGAADIIIGHLCIRHEMTPVNPGVGQ

SEQ ID NO: 132 - protein, 37 aa, Homo sapiens; identical to SEQ ID NO: 131
  DISULFID (10)..(23)

SEQ ID NO: 133 - protein, 39 aa, Homo sapiens
  DISULFID (1)..(11)
  DISULFID (24)..(39)
CQLSEDKLLACGEGAADIIIGHLCIRHEMTPVNPGVGQC

SEQ ID NO: 134 - protein, 41 aa, Homo sapiens
  DISULFID (1)..(12)
  DISULFID (25)..(41)
CCQLSEDKLLACGEGAADIIIGHLCIRHEMTPVNPGVGQCC

SEQ ID NO: 135 - protein, 41 aa, Homo sapiens
  DISULFID (1)..(11)
  DISULFID (12)..(25)
  DISULFID (26)..(41)
CQLSEDKLLACCGEGAADIIIGHLCCIRHEMTPVNPGVGQC

SEQ ID NO: 136 - protein, 9 aa, Homo sapiens
QLSEDKLLA

SEQ ID NO: 137 - protein, 14 aa, Homo sapiens
IRHEMTPVNPGVGQ

SEQ ID NO: 138 - protein, 11 aa, Homo sapiens
  DISULFID (1)..(11)
CQLSEDKLLAC

SEQ ID NO: 139 - protein, 14 aa, Homo sapiens
  DISULFID (1)..(14)
CGEGAADIIIGHLC

SEQ ID NO: 140 - protein, 16 aa, Homo sapiens
  DISULFID (1)..(16)
CIRHEMTPVNPGVGQC

SEQ ID NO: 141 - protein, 36 aa, Homo sapiens; identical to SEQ ID NO: 129

SEQ ID NO: 142 - protein, 34 aa, Homo sapiens
LSENKLLACGEGAANIIIGHLCIRHEMTPVNPGV

SEQ ID NO: 143 - protein, 34 aa, Homo sapiens; identical to SEQ ID NO: 2
  MISC_FEATURE (1)..(12): These residues may be D-amino acids.

SEQ ID NO: 144 - protein, 34 aa, Homo sapiens; identical to SEQ ID NO: 2
  MISC_FEATURE (28)..(34): These residues may be D-amino acids.

SEQ ID NO: 145 - protein, 35 aa, Homo sapiens
LSEDKLLACGEGAADIIIGHLCIRHEMTPVNPGVG

SEQ ID NO: 146 - protein, 7 aa, Homo sapiens
LSEIKLL

SEQ ID NO: 147 - protein, 14 aa, Homo sapiens
SEQRLPLSPLAPGN

SEQ ID NO: 148 - protein, 13 aa, Caenorhabditis elegans
  misc_feature (6)..(6): Xaa can be any naturally occurring amino acid
QRKLLXPPVAPGN

SEQ ID NO: 149 - protein, 16 aa, Homo sapiens
  misc_feature (15)..(15): Xaa can be any naturally occurring amino acid
TITKLIIFYGHLCSXH

SEQ ID NO: 150 - protein, 16 aa, Homo sapiens
GCGEGGGTLAPCLPGI

SEQ ID NO: 151 - protein, 17 aa, Rattus norvegicus
  misc_feature (12)..(13): Xaa can be any naturally occurring amino acid
ACGRGLLEAAPXXPGVG

SEQ ID NO: 152 - protein, 15 aa, Homo sapiens
  misc_feature (13)..(13): Xaa can be any naturally occurring amino acid
FCGSGVPAMAPVXPQ

SEQ ID NO: 153 - protein, 13 aa, Homo sapiens
PCGSASPFPPGVG

SEQ ID NO: 154 - protein, 7 aa, Caenorhabditis elegans
CGDGAAD

SEQ ID NO: 155 - protein, 18 aa, Homo sapiens
GEGAADDVLLRYCTKREI

SEQ ID NO: 156 - protein, 25 aa, Homo sapiens
  misc_feature (8)..(8): Xaa can be any naturally occurring amino acid
EGSSNLVXRHMVIKWQGXDSNPGVS

SEQ ID NO: 157 - protein, 11 aa, Xenopus sp.
SGTTIIVEHLI

SEQ ID NO: 158 - protein, 9 aa, Rattus norvegicus
YVDIFEGHL

SEQ ID NO: 159 - protein, 14 aa, Homo sapiens
PIRHRPRPYPPNVG

SEQ ID NO: 160 - protein, 8 aa, Homo sapiens
GTPIGPGV

SEQ ID NO: 161 - protein, 8 aa, Homo sapiens
GTPIGPGV

SEQ ID NO: 162 - protein, 9 aa, Drosophila melanogaster
FISSNPGVG

SEQ ID NO: 163 - protein, 15 aa, Xenopus sp.
LSDDRHMIVPVGPGV

SEQ ID NO: 164 - protein, 10 aa, Homo sapiens
DLLLACGEGS

SEQ ID NO: 165 - protein, 9 aa, Homo sapiens
AAEIVGGHE

SEQ ID NO: 166 - protein, 11 aa, Rattus norvegicus
IASIIIGHLWV

SEQ ID NO: 167 - protein, 8 aa, Rattus norvegicus
CIRHNPTG

SEQ ID NO: 168 - protein, 9 aa, Sus scrofa
MIAEFPGVG

SEQ ID NO: 169 - protein, 9 aa, Oryctolagus cuniculus
MIEYFPGVG

SEQ ID NO: 170 - protein, 27 aa, Homo sapiens
  misc_feature (13)..(13): Xaa can be any naturally occurring amino acid
KELACDAGGVHTXSESLCVRHTVSXVN

SEQ ID NO: 171 - protein, 16 aa, Gallus gallus
  misc_feature (4)..(4): Xaa can be any naturally occurring amino acid
GLLXCGTGAACTVSGH

SEQ ID NO: 172 - protein, 17 aa, Gallus gallus
RCGEGARGVGVGGGGVG

SEQ ID NO: 173 - protein, 9 aa, Homo sapiens
ISQIIVGHL

SEQ ID NO: 174 - protein, 9 aa, Homo sapiens
ISQIIVGHL

SEQ ID NO: 175 - protein, 11 aa, Mus musculus
ARVTIGPLCIR

SEQ ID NO: 176 - protein, 11 aa, Mus musculus
ARVTIGPLCIR

SEQ ID NO: 177 - protein, 9 aa, Homo sapiens
IIIYHLCII

SEQ ID NO: 178 - protein, 10 aa, Rattus norvegicus
CIRHSFLMLD

SEQ ID NO: 179 - protein, 13 aa, Homo sapiens
CISISNQPVNPRS

SEQ ID NO: 180 - protein, 13 aa, Homo sapiens
  misc_feature (3)..(3): Xaa can be any naturally occurring amino acid
CIXTQLGPVNPAL

SEQ ID NO: 181 - protein, 12 aa, Homo sapiens
GEGAADIIIGHL